(19) United States
(12) Patent Application Publication
(10) Pub. No.: US 2023/0242595 A1
(43) Pub. Date: Aug. 3, 2023

(54) ADAS COMPRISING BACTERIAL SECRETION SYSTEMS

(71) Applicant: Flagship Pioneering Innovations VI, LLC, Cambridge, MA (US)

(72) Inventors: Hok Hei TAM, Newton, MA (US); David Barry KOLESKY, Arlington, MA (US); Analise Zaunbrecher REEVES, Cambridge, MA (US); Caitlin Nicole SPAULDING, Boston, MA (US); Troy Patrick HUBBARD, Somerville, MA (US); Adam Barclay FISHER, Cambridge, MA (US); Kimberly A. HOMAN, Mill Valley, CA (US)

(21) Appl. No.: 18/011,008

(22) PCT Filed: Jun. 17, 2021
(86) PCT No.: PCT/US2021/037787

§ 371 Date: Dec. 16, 2022

Related U.S. Application Data
(60) Provisional application No. 63/147,296, filed on Feb. 9, 2021.
(60) Provisional application No. 63/040,457, filed on Jun. 17, 2020.

(51) Int. Cl. C07K 14/255; C07K 14/28; C07K 14/245; C12N 15/70

(52) U.S. Cl. CPC C07K 14/255; C07K 14/28; C07K 14/245; C12N 15/70

(57) ABSTRACT

The invention provides isolated achromosomal dynamic active systems (ADAS), including ADAS comprising secretion systems. These ADAS provided by the invention can be obtained by a variety of means. Various associated methods of making and using these ADAS are provided.

### CROSS-REFERENCES TO RELATED APPLICATIONS

This application claims priority to U.S. Patent Application No. 63/040,457, filed on Jun. 17, 2020, and U.S. Patent Application No. 63/147,296, filed on Feb. 9, 2021, the entire contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 15, 2021, is named 51296-040WO3_Sequence_Listing_6_15_21_ST25 and is 211,224 bytes in size.

### FIELD OF THE INVENTION

Provided herein are achromosomal dynamic active systems and methods of making and using the same.

### BACKGROUND

A need exists for delivery vectors capable of targeting cells and delivering biological agents, compositions containing such delivery vectors, and associated methods of delivering said vectors to cells, thereby modulating biological systems including animal, plant, and insect cells, tissues, and organisms. In particular, there is a need for delivery vectors that translocate cargoes (e.g., bacterial effectors or heterologous cargoes) into target cells.

### SUMMARY OF THE INVENTION

In some aspects, the disclosure features an achromosomal dynamic active system (ADAS) derived from a parent bacterial cell, the ADAS comprising a bacterial Type 3 secretion system (T3SS) that is heterologous to the parent bacterial cell.

In some embodiments, the parent bacterial cell is a Gram-negative bacterial cell.

In some embodiments, the parent bacterial cell does not comprise an endogenous T3SS.

In some embodiments, the parent bacterial cell is an E. coli cell. In some embodiments, the E. coli cell is a Nissle E. coli cell.

In some embodiments, the parent bacterial cell is a probiotic cell.

In some embodiments, the T3SS is from a genus of Table 6.

In some embodiments, the T3SS is a Salmonella T3SS, a Vibrio T355, an Escherichia T3SS, a Yersinia T3SS, a Shigella T3SS, a Pseudomonas T3SS, or a Chlamydia T3SS. In some embodiments, the Salmonella T3SS is a Salmonella enterica T3SS. In some embodiments, the Vibrio T3SS is a Vibrio parahaemolyticus T3SS. In some embodiments, the Escherichia T3SS is an enteropathogenic E. coli (EPEC) T3SS. In some embodiments, the Yersinia T3SS is a Yersinia enterocolitica T3SS. In some embodiments, the Shigella T3SS is a Shigella flexneri T3SS.

In some embodiments, the parent bacterial cell comprises one or more heterologous nucleotide sequences encoding the components of the T3SS. In some embodiments, the one or more nucleotide sequences encoding the components of the T3SS are carried on a vector. In some embodiments, the parent bacterial cell has been transiently transformed with the vector. In some embodiments, the parent bacterial cell has been stably transformed with the vector. In some embodiments, the parent bacterial cell further comprises a moiety that increases the level of the T3SS in the ADAS. In some embodiments, the moiety is a transcriptional activator of the one or more heterologous nucleotide sequences encoding a component of the T3SS.

In another aspect, the disclosure features an achromosomal dynamic active system (ADAS) derived from a parent bacterial cell, the ADAS comprising a bacterial Type 3 secretion system (T3SS) that is endogenous to the parent bacterial cell, wherein the parent bacterial cell has been modified to reduce the level of an endogenous protein or polypeptide capable of being secreted by the T3SS.

In some embodiments, the parent cell bacterial has been modified by deleting a transcriptional activator of the endogenous protein or polypeptide capable of being secreted by the T3SS.

In some embodiments, the parent bacterial cell is a Gram-negative bacterial cell.

In some embodiments, the parent bacterial cell is from a genus of Table 6.

In some embodiments, the parent bacterial cell is a Salmonella species, a Vibrio species, an Escherichia species, a Yersinia species, or a Shigella species. In some embodiments, the Salmonella species is Salmonella enterica. In some embodiments, the Vibrio species is a Vibrio parahaemolyticus. In some embodiments, the Escherichia species is an enteropathogenic E. coli (EPEC). In some embodiments, the Yersinia species is Yersinia enterocolitica. In some embodiments, the Shigella species is Shigella flexneri.

In some embodiments, the parent bacterial cell further comprises a moiety that increases the level of the T3SS in the ADAS. In some embodiments, the moiety is a transcriptional activator of a nucleotide sequence encoding a component of the T3SS.

In some embodiments, the parent bacterial cell has been modified to reduce the level of a negative regulator of a component of the T3SS. In some embodiments, a chromosomal locus encoding the negative regulator has been deleted from the parent bacterial cell.

In some embodiments, the parent bacterial cell has been modified to reduce the level of one or more of LPS; a metabolically non-essential protein; a toxin not associated with a T3SS; an endotoxin; a flagella; and a pillus.

In some embodiments, the ADAS further comprises at least one cargo, wherein the T3SS is capable of delivering the cargo to a target cell. In some embodiments, the delivery is to the cytoplasm of the target cell.

In some embodiments, the cargo is a protein or a polypeptide.

In some embodiments, the cargo is endogenously secreted by the T3SS.

In some embodiments, the ADAS or the parent bacterial cell has been modified to increase the level of the cargo in the ADAS.

In some embodiments, the cargo is not endogenously secreted by the T3SS.

In some embodiments, the cargo is endogenously secreted by a T3SS from a species other than the ADAS T3SS species.

In some embodiments, the cargo is endogenously secreted by a Type 4 secretion system (T4SS) or a Type 6 secretion system (T6SS).

In some embodiments, the cargo has been modified for delivery by the T3SS.

In some embodiments, the cargo is an enzyme, a DNA-modifying agent, a chromatin-remodeling agent, a gene editing agent, a nuclear targeting agent, a binding agent, an immunogenic agent, or a toxin. In some embodiments, the enzyme is a metabolic enzyme. In some embodiments, the gene editing agent is a component of a CRISPR system. In some embodiments, the nuclear targeting agent is a transcription factor. In some embodiments, the binding agent is an antibody or an antibody fragment. In some embodiments, the binding agent is a VHH molecule. In some embodiments, the immunogenic agent is an immunostimulatory agent. In some embodiments, the immunogenic agent is an immunosuppressive agent.

In some embodiments, the cargo has been modified by addition of a secretion signal. In some embodiments, the secretion signal is a sequence of Table 7.

In another aspect, the disclosure features a method for delivering a cargo to the cytoplasm of a target cell, the method comprising contacting the target cell with an ADAS of any one of the above aspects.

Other features and advantages of the invention will be apparent from the following Detailed Description and the Claims.

### BRIEF DESCRIPTION OF THE DRAWINGS

FIG. 1A is a micrograph showing human fibroblasts that have been incubated for 40 minutes with E. coli ADAS from strain M060 (lacking a T3SS). Nuclei are shown in blue; ADAS are shown in red; and actin is shown in white.

FIG. 1B is a micrograph showing human fibroblasts that have been incubated for 40 minutes with Salmonella typhimurium ADAS overexpressing a T3SS (strain M269). Nuclei are shown in blue; ADAS are shown in red; and actin is shown in white. Patches of heavy white staining demonstrate actin ruffling.

FIG. 2 is a photograph of an immunoblot showing expression of the T3SS-specific protein IpaD in the MACH320 E. coli strain, which expresses a Shigella flexneri T3SS, and in ADAS derived therefrom, in the presence or absence of the Shigella flexneri T3SS transcriptional activator.

FIG. 3 is a bar graph showing levels of delivery of an OspB-TEM beta-lactamase fusion protein to human dermal fibroblast, neonatal (HDFn) cells from control ADAS (derived from MACH434 parents) and E. coli ADAS having T3SS from S. flexneri (derived from MACH435 ADAS; T3SS+ ADAS). TEM1 Beta-lactamase transfer that occurs in a T3SS dependent manner (translocation) is expressed as the ratio of blue to green signal intensity (447 nm/518 nm), which corresponds to the amount of CCF4/AM dye cleaved by TEM1 Beta-lactamase. A cell-only preparation is provided as a background control. Asterisks indicate a significant (p<0.0001) increase in protein delivery in T3SS+ ADAS compared to control ADAS.

FIG. 4 is a photograph of an immunoblot showing expression of native (Shigella) and non-native (EPEC) FLAG-tagged effector proteins in ADAS derived from E. coli strains expressing a Shigella flexneri T3SS in the presence or absence of the T3SS activator VirB. Strains not having the T3SS, not having the activator, and/or not having the effector protein are provided as controls.

FIG. 5A is a graph showing the level of translocation of the T3SS cargo protein OspB-TEM1 into mammalian cells as a ratio of blue:green signal. Cells were treated with DH10b E. coli ADAS (Ec) comprising (T3+) or not comprising (T3−) a heterologous Shigella T3SS; CFT073 E. coli ADAS (CFT) comprising (T3+) or not comprising (T3−) a heterologous Shigella T3SS; or LT2 Salmonella enterica sp. Typhimurium ADAS (SeT) comprising or not comprising an endogenous T3SS. Untreated cells are shown as a control.

FIG. 5B is a graph showing the level of translocation of the T3SS cargo protein OspB-TEM1 into mammalian cells as a ratio of blue:green signal. Cells were treated with E. coli ADAS comprising a heterologous Shigella T3SS that were stored at 4° C. for 9 days or 36 days. Untreated cells are shown as a control.

### DETAILED DESCRIPTION OF THE INVENTION

### I. Definitions

As used herein, the term “endogenous Type 3 secretion system” or “endogenous T355” refers to a T3SS that is present on a cell (e.g., a parent cell), or an ADAS derived therefrom, and is naturally encoded by the cell (e.g., is encoded by a wild-type version of the cell). The T3SS may be expressed by the endogenous genes of the cell, and/or may be encoded and expressed by a synthetic construct in the cell. Expression or abundance of an endogenous T3SS may be increased, e.g., by the addition of a moiety that increases the abundance of the T3SS (e.g., a transcriptional activator of the T3SS) or reduction or removal of a negative regulator of expression of the T3SS.

As used herein, the term “heterologous Type 3 secretion system” or “heterologous T355” refers to a T3SS that is present on a cell (e.g., a parent cell), or an ADAS derived therefrom, and is not naturally encoded by the cell (e.g., is not encoded by a wild-type version of the cell). The cell may encode another T3SS, or may not encode any T3SS. In some embodiments, the T3SS is expressed by a synthetic construct in the cell.

As used herein, an “endogenous effector” of a secretion system (e.g., a T3SS, T4SS, or T6SS) is a moiety (e.g., a protein or polypeptide) that is naturally encoded by a cell from which the secretion system (e.g., T3SS) is derived (e.g., is encoded by a wild-type version of the cell) and is capable of being secreted by the secretion system. A secretion system and one or more of its endogenous effectors may be expressed in the cell in which they naturally occur or may be expressed heterologously, e.g., expressed by a cell that does not naturally encode the endogenous effector or the secretion system.

As used herein, an effector that is heterologous with respect to a secretion system (“heterologous effector”) is a moiety (e.g., a protein or polypeptide) that is not naturally encoded by a cell from which the secretion system (e.g., T3SS) is derived (e.g., is not encoded by a wild-type version of the cell) and is capable of being secreted by a secretion system of a cell from which the heterologous effector is derived. The effector may be capable of being secreted by the secretion system to which it is heterologous, or may be modified to be secreted by the secretion system to which it is heterologous. In some embodiments, the heterologous effector is an effector of a T4SS or a T6SS that is secreted by a T3SS.

As used herein, a “cargo” of a secretion system (e.g., T3SS) is a moiety that is capable of being secreted (e.g., delivered into the cytoplasm of a host cell or into the extracellular space) by the secretion system. A cargo may be an endogenous effector of the secretion system, a heterologous effector, or a moiety that is not naturally secreted by any secretion system. The cargo may be, e.g., a protein, or a polypeptide, e.g., an enzyme (e.g., a metabolic enzyme), a DNA-modifying agent (e.g., a component of a CRISPR system), a chromatin-remodeling agent, a gene editing agent, a nuclear targeting agent (e.g., a transcription factor), a binding agent (e.g., an antibody or an antibody fragment, e.g., a VHH molecule), an immunogenic agent (e.g., an immunostimulatory or immunosuppressive agent), or a toxin. The cargo may be modified to be secreted by the secretion system, e.g., by the addition of a secretion signal (e.g., an N-terminal secretion signal), e.g., a secretion signal provided in Table 8.

As used herein, the term “achromosomal dynamic system” or “ADAS” refers to a genome-free, non-replicating, enclosed membrane system comprising at least one membrane and having an interior volume suitable for containing a cargo (e.g., one or more of a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). In some embodiments, ADAS are minicells or modified minicells derived from a parent bacterial cell (e.g., a gram-negative or a gram-positive bacterial cell). In other aspects, ADAS are derived from parent cells by modifying the parent cell to remove the genome, and are substantially similar in size to the parent cell. ADAS may be derived from parent bacteria using any suitable method, e.g., genetic manipulation of the parent cell or exposure to a culture or condition that increases the likelihood of formation of bacterial minicells. Exemplary methods for making ADAS are those that disrupt the cell division machinery of the parent cell. In some embodiments, ADAS may comprise one or more endogenous or heterologous features of the parent cell surface, e.g., cell walls, cell wall modifications, flagella, or pilli, and/or one or more endogenous or heterologous features of the interior volume of the parent cell, e.g., nucleic acids, plasmids, proteins, small molecules, transcription machinery, or translation machinery. In other embodiments, ADAS may lack one or more features of the parent cell. In still other embodiments, ADAS may be loaded or otherwise modified with a feature not comprised by the parent cell.

As used herein, the term “highly active ADAS” refers to an ADAS having high work potential, e.g. an ADAS having the capability to do a significant amount of useful work. Work may be metabolic work, including chemical synthesis (e.g., of proteins, nucleic acids, lipids, carbohydrates, polymers, or small molecules), chemical modification (e.g., of proteins, nucleic acids, lipids, carbohydrates, polymers or small molecules), or transport (e.g., import, export, or secretion, e.g., secretion by a bacterial secretion system (e.g., T3SS)) under suitable conditions. In certain embodiments, highly active ADAS begin with a large pool of energy, e.g., energy in the form of ATP. In other embodiments, ADAS have the capacity to take up or generate energy/ATP from another source. Highly active ADAS may be identified, e.g., by having increased ATP concentration, increased ability to generate ATP, increased ability to produce a protein, increased rate or amount of production of a protein, and/or increased responsiveness to a biological signal, e.g., induction of a promoter.

As used herein, the term “parent bacterial cell” refers to a cell (e.g., a gram-negative or a gram-positive bacterial cell) from which an ADAS is derived. Parent bacterial cells are typically viable bacterial cells. The term “viable bacterial cell” refers to a bacterial cell that contains a genome and is capable of cell division. Preferred parent bacterial cells are derived from any of the strains in Table 4.

An ADAS composition or preparation that is “substantially free of” parent bacterial cells and/or viable bacterial cells is defined herein as a composition having no more than 500, e.g., 400, 300, 200, 150, 100 or fewer colony-forming units (CFU) per mL. An ADAS composition that is substantially free of parent bacterial cells or viable bacterial cells may include fewer than 50, fewer than 25, fewer than 10, fewer than 5, fewer than 1, fewer than 0.1, or fewer than 0.001 CFU/mL. including no bacterial cells.

The term “cell division topological specificity factor” refers to a component of the cell division machinery in a bacterial species that is involved in the determination of the site of the septum and functions by restricting the location of other components of the cell division machinery, e.g., restricting the location of one or more Z-ring inhibition proteins. Exemplary cell division topological specificity factors include minE, which was first discovered in E. coli and has since been identified in a broad range of gram-negative bacterial species and gram-positive bacterial species (Rothfield et al., Nature Reviews Microbiology, 3: 959-968, 2005). minE functions by restricting the Z-ring inhibition proteins minC and minD to the poles of the cell. A second exemplary cell division topological specificity factor is DivIVA, which was first discovered in Bacillus subtilis (Rothfield et al., Nature Reviews Microbiology, 3: 959-968, 2005).

The term “Z-ring inhibition protein” refers to a component of the cell division machinery in a bacterial species that is involved in the determination of the site of the septum and functions by inhibiting the formation of a stable FtsZ ring or anchoring such a component to a membrane. The localization of Z-ring inhibition proteins may be modulated by cell division topological specificity factors, e.g., minE and DivIVA. Exemplary Z-ring inhibition proteins include minC and minD, which were first discovered in E. coli and have since been identified in a broad range of gram-negative bacterial species and gram-positive bacterial species (Rothfield et al., Nature Reviews Microbiology, 3: 959-968, 2005). In E. coli and in other species, minC, minD, and minE occur at the same genetic locus, which may be referred to as the “min operon”, the minCDE operon, or the min or minCDE genetic locus.

As used herein, the term “reduction in the level or activity of a cell topological specificity factor,” refers to an overall reduction of any of 5%, 10%, 20%, 30%, 40%, 50%, 60%, 70%, 80%, 90%, 95%, 96%, 97%, 98%, 99%, or greater, in the level or activity of the cell topological specificity factor (e.g., protein or nucleic acid (e.g., gene or mRNA)), detected by standard methods, as compared to the level in a reference sample (for example, an ADAS produced from a wild-type cell or a cell having a wild-type minCDE operon or wild-type div/VA gene), a reference cell (for example, a wild-type cell or a cell having a wild-type minC, minD, minE, div/VA, or minCDE gene or operon), a control sample, or a control cell. In some embodiments, a reduced level or activity refers to a decrease in the level or activity in the sample which is at least about 0.9×, 0.8×, 0.7×, 0.6×, 0.5×, 0.4×, 0.3×, 0.2×, 0.1×, 0.05×, or 0.01× the level or activity of the cell topological specificity factor in a reference sample, reference cell, control sample, or control cell.

As used herein, the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y) where X is the number of nucleotides or amino acids scored as identical matches by a sequence alignment program (e.g., BLAST) in that program's alignment of A and B, and where Y is the total number of nucleotides or amino acids in B. In some embodiments, sequence identity, for example, in homologues of MinE or DivIVA proteins will have at least about 40%, 50%, 60%, 70%, 80%, 85%, 90%, or even 95% or greater amino acid or nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% or greater amino acid sequence or nucleic acid identity, to a native sequence MinE (or minE) or DivIVA (or div/VA) sequence as disclosed herein.

The phrases “modulating a state of a cell” as used herein, refers to an observable change in the state (e.g., the transcriptome, proteome, epigenome, biological effect, or health or disease state) of the cell (e.g., an animal, plant, or insect cell) as measured using techniques and methods known in the art for such a measurement, e.g., methods to measure the level or expression of a protein, a transcript, an epigenetic mark, or to measure the increase or reduction of activity of a biological pathway. Modulating the state of the cell may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating the state of the cell involves increasing a parameter (e.g., the level or expression of a protein, a transcript, or activity of a biological pathway) of the cell. Increasing the state of the cell may result in an increase of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating the state of involves decreasing a parameter (e.g., the level or expression of a protein, a transcript, or activity of a biological pathway) of the cell. Decreasing the state of the cell may result in a decrease of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration).

As used herein, the term “heterologous” means not native to a cell or composition in its naturally-occurring state. In some embodiments “heterologous” refers to a molecule; for example, a cargo or payload (e.g., a polypeptide, a nucleic acid such as a protein-encoding RNA or tRNA, or small molecules) or a structure (e.g., a plasmid or a gene-editing system) that is not found naturally in an ADAS or the parent bacteria from which it is produced (e.g., a gram-negative or gram positive bacterial cell).

### II. Compositions

A. ADAS Comprising a Secretion System

In certain embodiments, an ADAS provided by the invention comprises a bacterial secretion system (e.g., an endogenous bacterial secretion system or a heterologous secretion system). A “bacterial secretion system” is a protein, or protein complex, that can export a cargo from the cytoplasm of a bacterial cell (or, for example, an ADAS derived therefrom) into: the extracellular space, the periplasmic space of a gram-negative bacterium, or the intracellular space of another cell. In some embodiments, the bacterial secretion system works by an active (e.g., ATP-dependent or PMF-dependent) process, and in certain embodiments the bacterial secretion system comprises a tube or a spike spanning the host cell (or ADAS) to a target cell. In other embodiments the bacterial secretion system is a transmembrane channel. Exemplary bacterial secretion systems include T3SS and T4SS (and T3/T4SS, as defined, below), which are tube-containing structures where the cargo traverses through the inside of a protein tube and T6SS, which carries the cargo at the end of a spike. Other exemplary bacterial secretion systems include T1SS, T2SS, T5SS, T7SS, Sec, and Tat, which are transmembrane.

In some aspects, the disclosure features an achromosomal dynamic active system (ADAS) derived from a parent bacterial cell, the ADAS comprising a bacterial Type 3 secretion system (T3SS) that is heterologous to the parent bacterial cell.

In some embodiments, the parent bacterial cell is a Gram-negative bacterial cell.

In some embodiments, the parent bacterial cell does not comprise an endogenous T3SS.

In some embodiments, the parent bacterial cell is an E. coli cell. In some embodiments, the E. coli cell is a Nissle E. coli cell.

In some embodiments, the parent bacterial cell is a probiotic cell.

In some embodiments, the T3SS is from a genus of Table 6.

In some embodiments, the T3SS is a Salmonella T3SS, a Vibrio T3SS, an Escherichia T3SS, a Yersinia T3SS, a Shigella T3SS, a Pseudomonas T3SS, or a Chlamydia T3SS. In some embodiments, the Salmonella T3SS is a Salmonella enterica T3SS. In some embodiments, the Vibrio T3SS is a Vibrio parahaemolyticus T3SS. In some embodiments, the Escherichia T3SS is an enteropathogenic E. coli (EPEC) T3SS. In some embodiments, the Yersinia T3SS, is a Yersinia enterocolitica T3SS. In some embodiments, the Shigella T3SS is a Shigella flexneri T3SS.

In some embodiments, the parent bacterial cell comprises one or more heterologous nucleotide sequences encoding the components of the T3SS. In some embodiments, the one or more nucleotide sequences encoding the components of the T3SS are carried on a vector. In some embodiments, the parent bacterial cell has been transiently transformed with the vector. In some embodiments, the parent bacterial cell has been stably transformed with the vector. In some embodiments, the parent bacterial cell further comprises a moiety that increases the level of the T3SS in the ADAS. In some embodiments, the moiety is a transcriptional activator of the one or more heterologous nucleotide sequences encoding a component of the T3SS.

In another aspect, the disclosure features an achromosomal dynamic active system (ADAS) derived from a parent bacterial cell, the ADAS comprising a bacterial Type 3 secretion system (T3SS) that is endogenous to the parent bacterial cell, wherein the parent bacterial cell has been modified to reduce the level of an endogenous protein or polypeptide capable of being secreted by the T3SS.

In some embodiments, the parent cell bacterial has been modified by deleting a transcriptional activator of the endogenous protein or polypeptide capable of being secreted by the T3SS.

In some embodiments, the parent bacterial cell is a Gram-negative bacterial cell.

In some embodiments, the parent bacterial cell is from a genus of Table 6.

In some embodiments, the parent bacterial cell is a Salmonella species, a Vibrio species, an Escherichia species, a Yersinia species, or a Shigella species. In some embodiments, the Salmonella species is Salmonella enterica. In some embodiments, the Vibrio species is a Vibrio parahaemolyticus. In some embodiments, the Escherichia species is an enteropathogenic E. coli (EPEC). In some embodiments, the Yersinia species is Yersinia enterocolitica. In some embodiments, the Shigella species is Shigella flexneri.

In some embodiments, the parent bacterial cell further comprises a moiety that increases the level of the T3SS in the ADAS. In some embodiments, the moiety is a transcriptional activator of a nucleotide sequence encoding a component of the T3SS.

In some embodiments, the parent bacterial cell has been modified to reduce the level of a negative regulator of a component of the T3SS. In some embodiments, a chromosomal locus encoding the negative regulator has been deleted from the parent bacterial cell.

In some embodiments, the parent bacterial cell has been modified to reduce the level of one or more of LPS; a metabolically non-essential protein; a toxin not associated with a T3SS; an endotoxin; a flagella; and a pillus.

In some embodiments, the ADAS further comprises at least one cargo, wherein the T3SS is capable of delivering the cargo to a target cell. In some embodiments, the delivery is to the cytoplasm of the target cell.

In some embodiments, the cargo is a protein or a polypeptide.

In some embodiments, the cargo is endogenously secreted by the T3SS.

In some embodiments, the ADAS or the parent bacterial cell has been modified to increase the level of the cargo in the ADAS.

In some embodiments, the cargo is not endogenously secreted by the T3SS.

In some embodiments, the cargo is endogenously secreted by a T3SS from a species other than the ADAS T3SS species.

In some embodiments, the cargo is endogenously secreted by a Type 4 secretion system (T4SS) or a Type 6 secretion system (T6SS).

In some embodiments, the cargo has been modified for delivery by the T3SS.

In some embodiments, the cargo is an enzyme, a DNA-modifying agent, a chromatin-remodeling agent, a gene editing agent, a nuclear targeting agent, a binding agent, an immunogenic agent, or a toxin. In some embodiments, the enzyme is a metabolic enzyme. In some embodiments, the gene editing agent is a component of a CRISPR system. In some embodiments, the nuclear targeting agent is a transcription factor. In some embodiments, the binding agent is an antibody or an antibody fragment. In some embodiments, the binding agent is a VHH molecule. In some embodiments, the immunogenic agent is an immunostimulatory agent. In some embodiments, the immunogenic agent is an immunosuppressive agent.

In some embodiments, the cargo has been modified by addition of a secretion signal. In some embodiments, the secretion signal is a sequence of Table 7.

In another aspect, the disclosure features a method for delivering a cargo to the cytoplasm of a target cell, the method comprising contacting the target cell with an ADAS of any one of the above aspects.

In some embodiments, the ADAS comprises a cargo, wherein the cargo comprises a moiety that directs export by the bacterial secretion system, e.g., in some embodiments the moiety is Pho/D, Tat, or a synthetic peptide signal.

In certain embodiments, the ADAS provided by the invention are two-membrane ADAS. In more particular embodiments the two-membrane ADAS further comprises a bacterial secretion system. In still more particular embodiments, the bacterial secretion system is selected from T3SS, T4SS, T3/4SS, or T6SS, optionally wherein the T3SS, T4SS, T3/4SS, or T6SS have an attenuated or non-functional effector that does not affect fitness of a target cell.

ADAS provided by the invention, in some embodiments, include a bacterial secretion system. In some embodiments, the bacterial secretion system is capable of exporting a cargo across the ADAS outer membrane into a target cell, such as an animal, fungal, bacterial, or plant cell, such as T3SS, T4SS, T3/T4SS, or T6SS.

In more particular embodiments, the bacterial secretion system is a T3/4SS. A “T3/4SS” is a secretion system based on T3SS or T4SS, including hybrid systems as well as unmodified versions, which forms a protein tube between a bacterium (or ADAS) and a target cell, connecting the two and delivering one or more effectors. The target cell can be an animal, plant, fungi, or bacteria. In some embodiments a T3/4SS includes an effector, which may be a modified effector. Examples of T3SS systems include the Salmonella SPI-1 system, the EHEC coli ETT1 system, the Xanthamonas Citri/Campestri T3SS system, and the Pseudomonas syringae T3SS system. Examples of T455 systems include the Agrobacterium Ti plasmid system, Helicobacter pylori T4SS. In certain embodiments, the T3/455 has modified effector function, e.g., an effector selected from SopD2, SopE, Bop, Map, Tir, EspB, EspF, NleC, NleH2, or NleE2. In more particular embodiments, the modified effector function is for intracellular targeting such as translocation into the nucleus, golgi, mitochondria, actin, microvilli, ZO-1, microtubules, or cytoplasm. In still more particular embodiments, the modified effector function is nuclear targeting based on NleE2 derived from E. Coli. In other particular embodiments, the modified effector function is for filopodia formation, tight junction disruption, microvilli effacement, or SGLT-1 inactivation. In other embodiments, an ADAS provided by the invention comprising a bacterial secretion system comprises a T6SS. In some embodiments, the T6SS, in its natural host, targets a bacterium and contains an effector that kills the bacteria. In certain particular embodiments, the T6SS is derived from P. putida K1-T6SS and, optionally, wherein the effector comprises the amino acid sequence of Tke2 (Accession AUZ59427.1), or a functional fragment thereof. In other embodiments, the T6SS, in its natural host, targets a fungi and contains an effector that kills fungi, e.g., the T6SS is derived from Serratia Marcescens and the effectors comprise the amino acid sequences of: Tfe1 (Genbank: SMDB11_RS05530) or Tfe2 (Genbank: SMDB11_RS05390).

In other embodiments of an ADAS provided by the invention that contains a bacterial secretion system, the bacterial secretion system is capable of exporting a cargo extracellularly. In certain more particular embodiments, the bacterial secretion system is T1SS, T2SS, T5SS, T7SS, Sec, or Tat.

B. ADAS and Highly Active ADAS

The invention is based, at least in part, on Applicant's discovery of achromosomal dynamic active systems (ADAS), including highly active ADAS, which are able to provide a wide array of functions in a large number of environments. An “ADAS” is a genome-free, non-replicating, enclosed membrane system comprising at least one membrane (in some embodiments, two membranes, where the two membranes are non-intersecting) and having an interior volume suitable for containing a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)).

In some embodiments, ADAS are minicells or modified minicells derived from a parent bacterial cell (e.g., a gram-negative or a gram-positive bacterial cell). ADAS may be derived from parent bacteria using any suitable method, e.g., genetic manipulation of the parent cell or exposure to a culture or condition that increases the likelihood of formation of bacterial minicells.

In some embodiments, an ADAS has a major axis cross section between about 100 nm-500 μm (e.g., in certain embodiments, about: 100-600 nm, such as 100-400 nm; or between about 0.5-10 μm, and 10-500 μm). In certain embodiments, an ADAS has a minor axis cross section between about: 0.001, 0.01, 0.1, 1, 2, 3, 4, 5, 6, 7, 8, 9, 10, 11, 12, 13, 14, 15, 20, 30, 40, 50, 60, 70, 80, 90, up to 100% of the major axis. In certain embodiments, an ADAS has an interior volume of between about: 0.001-1 μm³, 0.3-5 μm³, 5-4000 μm³, or 4000-50×10⁷ μm³. In some embodiments, the ADAS is substantially similar in size to the parent cell, e.g., has a size (e.g., interior volume, major axis cross-section, and/or minor axis cross section) that is about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the size of the parent cell, has a size that is identical to that of the parent cell, or has a size that is about 101%, 102%, 103%, 104%, 105%, 106%, 107%, 108%, 109%, or 110% of the size of the parent cell.

In some embodiments, the invention provides highly active ADAS. A “highly active” ADAS is an ADAS with high work potential, e.g. an ADAS having the capability to do a significant amount of useful work. Work may be defined as, e.g., metabolic work, including chemical synthesis (e.g., synthesis of proteins, nucleic acids, lipids, carbohydrates, polymers, or small molecules), chemical modification (e.g., modification of proteins, nucleic acids, lipids, carbohydrates, polymers or small molecules), or transport (e.g., import, export, or secretion) under suitable conditions. In some embodiments, highly active ADAS begin with a large pool of energy, e.g., energy in the form of adenosine triphosphate (ATP). In other embodiments, ADAS have the capacity to take up or generate energy (e.g., ATP) from another source.

The term “ADAS provided by the invention” encompasses all embodiments of ADAS described herein, including, in particular embodiments, highly active ADAS, the set of which can be referenced as “highly active ADAS provided by the invention”, which is a subset of the ADAS provided by the invention.

In one aspect, the invention provides a composition comprising a plurality of highly active achromosomal dynamic active systems (ADAS), wherein the ADAS have an initial ATP concentration of at least 1 mM and wherein the composition is substantially free of viable bacterial cells.

In another aspect, the invention provides a composition comprising a plurality of highly active achromosomal dynamic active systems (ADAS), wherein the ADAS have an initial ATP concentration of at least 3 mM and wherein the composition is substantially free of viable bacterial cells.

In some embodiments, a highly active ADAS has an initial ATP concentration of at least 1 nM, 1.1. nM, 1.2 nM, 1.3 nM, 1.4 mM, 1.5 mM, 1.6 mM, 2 mM, 2.5 mM, 3 nM, 3.5 nM, 4 mM, 5 mM, 10 mM, 20 mM, 30 mM, or 50 mM. ATP concentration can be evaluated by a variety of means including, in certain embodiments, a BacTiter-Glo™ assay (Promega) on lysed ADAS.

High activity may be additionally or alternatively assessed as the rate or amount of increase in ATP concentration in an ADAS over time. In some embodiments, the ATP concentration of an ADAS is increased by at least 50%, at least 60%, at least 75%, at least 100%, at least 150%, at least 200%, or more than 200% following incubation under suitable conditions, e.g., incubation at 37° C. for 12 hours. In certain embodiments, a highly active ADAS has a rate of ATP generation greater than about: 0.000001, 0.00001, 0.0001, 0.001, 0.01, 0.05, 0.1, 0.5, 1.0, 2, 3, 5, 10, 15, 20, 30, 40, 50, 75, 100, 200, 300, 500, 1000, 10000 ATP/sec/nm² for at least about: 1 hour, 2 hours, 3 hours, 4 hours, 5 hours, 6 hours, 12 hours, 1 day, 2 days, 4 days, 1 week, or two weeks.

In other aspects, high activity is assessed as a rate of decrease in ATP concentration over time. In some embodiments, ATP concentration may decrease less rapidly in ADAS that are highly active than in ADAS that are not highly active. In some embodiments, the drop in ATP concentration in an ADAS or an ADAS composition at 24 hours after preparation is less than about 50% (e.g., less than about: 45, 40, 35, 30, 25, 20, 15, 10, or 5%) compared to the initial ATP concentration (e.g., ATP per cell volume), e.g., as measured using a BacTiter-Glo™ assay (Promega).

High activity may be additionally or alternatively assessed as lifetime index of an ADAS. The lifetime index is calculated as the ratio of the rate of GFP production at 24 hours vs. 30 minutes. In some embodiments, a highly active ADAS has a lifetime index of greater than about: 0.13, 0.14, 0.15, 0.16, 0.18, 0.2, 0.25, 0.3, 0.35, 0.45, 0.5, 0.60, 0.70, 0.80, 0.90, 1.0 or more. In more particular embodiments, lifetime index is measured in an ADAS containing a functional GFP plasmid with a species-appropriate promoter in which GFP concentration is measured relative to number of ADAS, average number of plasmids per ADAS, and solution volume with a plate reader at 30 minutes and 24 hours.

In some aspects, the ADAS produces a protein, e.g., a heterologous protein. In some aspects, high activity is assessed as a rate, amount, or duration of production of a protein or a rate of induction of expression of the protein (e.g., responsiveness of an ADAS to a signal). For example, the ADAS may comprise a plasmid, the plasmid comprising an inducible promoter and a nucleotide sequence encoding the heterologous protein, wherein contacting the ADAS with an inducer of the inducible promoter under appropriate conditions results in production of the heterologous protein. In some aspects, the production of the heterologous protein is increased by at least 1.6-fold in an ADAS, e.g., a highly active ADAS, that has been contacted with the inducer relative to an ADAS that has not been contacted with the inducer. For example, in some embodiments, the production of the heterologous protein is increased by at least 1.5-fold, 1.75-fold, 2-fold, 3-fold, 4-fold, 5-fold, 10-fold, or more than 10-fold in an ADAS, e.g., a highly active ADAS, that has been contacted with the inducer. In some embodiments, the rate of production of the heterologous protein by a highly active ADAS reaches a target level within a particular duration following the contacting of the ADAS with the inducer, e.g., within 5 minutes, 10 minutes, 15 minutes, 30 minutes, 45 minutes, 1 hour, 2 hours, 3 hours, or more than 3 hours. In some embodiments, a protein (e.g., a heterologous protein) is produced at a rate of at least 0.1 femtograms per hour per highly active ADAS, e.g, at least 0.2 0.4, 0.6, 0.8, 1, 2, 4, 6, 8, 10, 25, 50, 100, 250, 500, 1000, 2000, 3000, or 3500 fg/hour per ADAS. In some embodiments, high activity of an ADAS is assessed as a duration for which a protein is produced. A highly active ADAS may produce a protein (e.g. a heterologous protein) for a duration of at least 2 hours, at least 4 hours, at least 8 hours, at least 12 hours, at least 24 hours, at least 48 hours, or longer than 48 hours.

C. ADAS and Highly Active ADAS Derived from Parent Bacteria Deficient in a Cell Division Topological Specificity Factor

ADAS may be derived from bacterial parent cells, as described herein.

In some aspects, the invention provides an ADAS and/or a composition comprising a plurality of ADAS derived from a parent bacterium having a reduction in a level, activity, or expression of a cell division topological specificity factor.

In some aspects, the invention provides a composition comprising a plurality of ADAS, wherein the ADAS do not comprise a cell division topological specificity factor and wherein the composition is substantially free of viable bacterial cells.

In some aspects, the invention provides a composition comprising a plurality of ADAS, the composition being substantially free of viable bacterial cells, and being produced by a process comprising: (a) making, providing, or obtaining a plurality of parent bacteria having a reduction in the level or activity of a cell division topological specificity factor; (b) exposing the parent bacterium to conditions allowing the formation of a minicell, thereby producing the highly active ADAS; and (c) separating the ADAS from the parent bacterium, thereby producing a composition that is substantially free of viable bacterial cells.

In some embodiments of the above aspects, the cell division topological specificity factor is a polypeptide having an amino acid sequence with at least 20% identity to an E. coli minE polypeptide (SEQ ID NO: 46), e.g., at least 30%, 40%, 50%, 60%, 70%, 80%, 90%, 95%, 98%, or 99% identity to SEQ ID NO: 46. In some embodiments, the cell division topological specificity factor comprises the amino acid sequence of SEQ ID NO: 46. In some embodiments, the cell division topological specificity factor is a minE polypeptide. Exemplary species having minE polypeptides are provided in Table 1 and in Rothfield et al., Nature Reviews Microbiology, 3: 959-968, 2005.

In some embodiments, the parent bacterium is E. coli and the minE polypeptide is E. coli minE. In other embodiments, the parent bacterium is Salmonella typhimurium and the minE polypeptide is S. typhimurium minE. In yet other embodiments, the parent bacterium is an Escherichia, Acinetobacter, Agrobacterium, Anabaena, Anaplasma, Aquifex, Azoarcus, Azospirillum, Azotobacter, Bartonella, Bordetella, Bradyrhizobium, Brucella, Buchnera, Burkholderia, Candidatus, Chromobacterium, Coxiella, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylobacterium, Methylococcus, Neisseria, Nitrosomonas, Nostoc, Photobacterium, Photorhabdus, Phyllobacterium, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Rhizobium, Rickettsia, Symbiobacterium, or Thermoanaerobacter bacterium and the cell division topological specificity factor is the endogenous minE or DivIVA of the parent bacterium.

In some embodiments of the above aspects, the cell division topological specificity factor is a polypeptide having an amino acid sequence with at least 20% identity to a Bacillus subtilis DivIVA polypeptide (SEQ ID NO: 49), e.g., at least 30%, 40%, 50%, 60%, 70%, 80%, 90%, 95%, 98%, or 99% identity to SEQ ID NO: 49. In some embodiments, the cell division topological specificity factor comprises the amino acid sequence of SEQ ID NO: 49. In some embodiments, the cell division topological specificity factor is a DivIVA polypeptide. Exemplary species having DivIVA polypeptides are provided in Table 1 and in Rothfield et al., Nature Reviews Microbiology, 3: 959-968, 2005. In some embodiments, the parent bacterium is Bacillus subtilis and the cell division topological specificity factor is B. subtilis DivIVA.

In some embodiments, the ADAS or parent bacterium having the reduction in a level or activity of the cell division topological specificity factor also has a reduction in a level of one or more Z-ring inhibition proteins.

In some embodiments, the Z ring inhibition protein is a polypeptide having an amino acid sequence with at least 20% identity to an E. coli minC polypeptide (SEQ ID NO: 47), e.g., at least 30%, 40%, 50%, 60%, 70%, 80%, 90%, 95%, 98%, or 99% identity to SEQ ID NO: 47. In some embodiments, the Z ring inhibition protein comprises the amino acid sequence of SEQ ID NO: 47. In some embodiments, the Z ring inhibition protein is a minC polypeptide.

In some embodiments, the Z ring inhibition protein is a polypeptide having an amino acid sequence with at least 20% identity to an E. coli minD polypeptide (SEQ ID NO: 48), e.g., at least 30%, 40%, 50%, 60%, 70%, 80%, 90%, 95%, 98%, or 99% identity to SEQ ID NO: 48. In some embodiments, the Z ring inhibition protein comprises the amino acid sequence of SEQ ID NO: 48. In some embodiments, the Z ring inhibition protein is a minD polypeptide.

In some embodiments, the ADAS or parent bacterium has a reduction in the level, activity, or expression of at least two Z-ring inhibition proteins. In some embodiments, the ADAS or parent bacterium has a reduction in expression of a minC polypeptide and a minD polypeptide. In some embodiments, the ADAS or parent bacterium has a reduction in expression of a minC polypeptide, a minD polypeptide, and a minE polypeptide, e.g., a deletion of the minCDE operon (ΔminCDE).

A reduction in the level, activity, or expression of a cell division topological specificity factor or a Z-ring inhibition protein, e.g., a reduction in an ADAS or a reduction in a parent bacterial cell, may be achieved using any suitable method. For example, in some embodiments, the reduction in the level or activity is caused by a loss-of-function mutation, e.g., a gene deletion. In some embodiments, the loss-of-function mutation is an inducible loss-of-function mutation and loss of function is induced by exposing the parent cell to an inducing condition, e.g., the inducible loss-of-function mutation is a temperature-sensitive mutation and wherein the inducing condition is a temperature condition.

In some embodiments, the parent cell has a deletion of the minCDE operon (ΔminCDE) or homologous operon.

D. ADAS Comprising a Cargo

In some embodiments, an ADAS provided by the invention includes a cargo contained in the interior of the ADAS. A cargo may be any moiety disposed in the interior of an ADAS (e.g., encapsulated by the ADAS) or conjugated to the surface of the ADAS. In some embodiments, the cargo comprises a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP) or a combination of the foregoing. In some aspects, the cargo is delivered by the secretion system (e.g., T3SS). In other aspects, the cargo Is not delivered by the T3SS.

In some embodiments, the nucleic acid is a DNA, an RNA, or a plasmid. In some embodiments, the nucleic acid (e.g., DNA, RNA (e.g., mRNA, ASO, circular RNA, siRNA, shRNA, tRNA, dsRNA, or a combination thereof), or plasmid) encodes a protein. In some embodiments, the protein is transcribed and/or translated in the ADAS. In some embodiments, the nucleic acid inhibits translation of a protein or polypeptide, e.g., is an siRNA or an antisense oligonucleotide (ASO).

In some embodiments, the cargo is an agent that can modulate the microbiome of the target organism (e.g., a human, animal, plant, or insect microbiome), e.g., a polysaccharide, an amino acid, an anti-microbial agent (e.g., e.g., an anti-infective or antimicrobial peptide, protein, and/or natural product), a short chain fatty acid, or a combination thereof. In some examples, the agent that can modulate the host microbiome is a probiotic agent.

In some embodiments, the cargo is an enzyme. In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered.

In certain embodiments, the cargo is modified for improved stability compared to an unmodified version of the cargo. “Stability” of a cargo is a unitless ratio of half-life of unmodified cargo and modified cargo half-life, as measured in the same environmental conditions. In some embodiments the environment is experimentally controlled, e.g., a simulated body fluid, RNAse free water, cell cytoplasm, extracellular space, or “ADAS plasm” (i.e., the content of the interior volume of an ADAS, e.g., after lysis). In some applications it is an agricultural environment, e.g., variable field soil, river water, or ocean water.

In other embodiments, the environment is an actual or simulated: animal gut, animal skin, animal reproductive tract, animal respiratory tract, animal blood stream, or animal extracellular space. In certain embodiments, the ADAS does not substantially degrade the cargo.

In certain embodiments, the cargo comprises a protein. In certain embodiments, the protein has stability greater than about: 1.01, 1.1, 10, 100, 1000, 10000, 100000, 100000, 10000000 in cell cytoplasm or other environments. The protein can be any protein, including growth factors; enzymes; hormones; immune-modulatory proteins; antibiotic proteins, such as antibacterial, antifungal, insecticide, proteins, etc.; targeting agents, such as antibodies or nanobodies, etc. In some embodiments, the protein is a hormone, e.g., paracrine, endocrine, autocrine.

In some embodiments, the cargo comprises a plant hormone, such as abscisic acid, auxin, cytokinin, ethylene, gibberellin, or a combination thereof.

In some embodiments, the cargo is an anti-inflammatory agent, e.g., a cytokine (e.g., a heterologously expressed anti-inflammatory cytokine or mutein thereof (e.g., IL-10, TGF-Beta, IL-22, IL-2) or antibody (e.g., an antibody or antibody fragment targeting tumor necrosis factor (TNF) (e.g., an anti-TNF antibody); an antibody or antibody fragment targeting IL-12 (e.g., an anti-IL-12 antibody); or an antibody or antibody fragment targeting IL-23 (e.g., an anti-IL-23 antibody).

In certain embodiments, the cargo is an immune modulator. Immune modulators include, e.g., immune stimulators; checkpoint inhibitors (e.g., inhibitors of PD-1, PD-L1, or CTLA-4); chemotherapeutic agents; immune suppressors; antigens; super antigens; and small molecules (e.g., cyclosporine A, cyclic dinucleotides (CDNs), or STING agonists (e.g., MK-1454)). In some embodiments, the immune modulator is a moiety that induces tolerance in a subject, e.g., an allergen, a self-antigen (e.g., a disease-associated self-antigen), or a microbe-specific antigen. In some embodiments, the immune modulator is a vaccine, e.g., an antigen from a pathogen (e.g., a virus (e.g., a viral envelope protein) or a bacteria). In some embodiments, the antigen is a cancer neo-antigen. In some embodiments, the pathogen is a coronavirus, e.g., SARS-CoV-2 In some embodiments, the cargo an adjuvant, e.g., an immunomodulatory molecule or a molecule that alters the compartmentalization, presentation, or profile of one or more co-stimulatory molecules associated with a vaccine antigen. In some examples, the adjuvant is an activator of an immune pathway upstream of a desired immune response (e.g., an activator of an innate immune pathway upstream of an adaptive immune response). In other examples, the adjuvant enhances the presentation of an antigen on an immune cell or immune moiety (e.g., MHC class 1) in the target organism. In some examples, the adjuvant is listeriolysin 0 (LLO). In some embodiments, an ADAS comprises an antigen and one or more adjuvants.

In some embodiments, the cargo is an agent for treatment or prevention of a cancer, e.g., an agent that decreases the likelihood that a patient will develop a cancer or an agent that treats a cancer (e.g., an agent that increases progression-free survival and/or overall survival in an individual having a cancer).

Agents for the prevention of cancer include, but are not limited to anti-inflammatory agents and growth inhibitors. Agents for the treatment of cancer (e.g., a solid tumor cancer) include, but are not limited to anti-inflammatory agents, growth inhibitors, chemotherapy agents, immunotherapy agents, anti-cancer antibodies or antibody fragments (e.g., antibodies or antibody fragments targeting cancer antigens (e.g., cancer neo-antigens)), cancer vaccines (e.g., vaccines comprising a cancer neo-antigen), agents that induce autophagy (e.g., activators such as listeria-lysin-o), cytotoxins, inflammasome inhibiting agents, immune checkpoint inhibitors (e.g., inhibitors of PD-1, PD-L1, or CTLA-4), transcription factor inhibitors, and agents that disrupt the cytoskeleton.

In some embodiments, the cargo is an enzyme. The enzyme may be an enzyme that performs a catalytic activity in a target cell or organism (e.g., in a human, animal, plant, or insect). In some embodiments, the catalytic activity is extracellular matrix (ECM) digestion (e.g., the enzyme is hyaluronidase and the catalytic activity is ECM digestion) or removal of toxins. In some embodiments, the enzyme is an enzyme replacement therapy, e.g., is phenylalanine hydroxylase. In some embodiments, the enzyme is a UDP-glucuronosyltransferase. In some embodiments, the enzyme has hepatic enzymatic activity (e.g., porphobilinogen deaminase (PBGD), e.g., human PBGD (hPBGD)). In some embodiments, the enzyme is a protease, oxidoreductase, or a combination thereof.

In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered. In some embodiments, the enzyme is diadenylate cyclase A, the substrate is ATP, and the target product is cyclic-di-AMP.

In some embodiments, the enzyme is chemically conjugated to the ADAS membrane, optionally via a linker to the exterior membrane.

Alternatively, the cargo may be a nucleic acid that encodes any of the enzymes described herein.

In some embodiments, the cargo is an agent that activates or inhibits autophagic processes (e.g. an activator such as listeria-lysin-o or an inhibitor such as IcsB).

In some embodiments, the cargo is an anti-infective agent, e.g., an anti-microbial agent, e.g., an anti-infective or antimicrobial peptide, protein, and/or natural product.

In some embodiments, the cargo is a protein that modulates host transcriptional response e.g., a transcription factor; a protein that promotes host cell growth, e.g., a growth factor; or a protein that inhibits protein function, e.g., a nanobody. In some embodiments, the transcription factor is a human transcription factor.

For ADAS comprising cargo, in some embodiments, the cargo is an RNA, such as circular RNA, mRNA, siRNA, shRNA, ASO, tRNA, dsRNA, or a combination thereof. In certain embodiments, the RNA has stability greater than about: 1.01, 1.1, 10, 100, 1000, 10000, 100000, 100000, 10000000, e.g., in ADAS plasm. The RNA cargo can be stabilized, in certain embodiments, e.g., with an appended step-loop structure, such as a tRNA scaffold. For example, non-human tRNALys3 and E. coli tRNAMet (Nat. Methods, Ponchon 2007). Both have been well characterized and expressed recombinantly. However, a variety of other types could be used as well, such as aptamers, lncRNA, ribozymes, etc. RNA can also be stabilized where the ADAS is obtained from a parental strain null (or hypomorphic) for one or more ribonucleases.

In some particular embodiments, the RNA is a protein-coding mRNA. In more particular embodiments, the protein-coding mRNA encodes an enzyme (e.g., and enzyme that imparts hepatic enzymatic activity, such as human PBGD (hPBGD) mRNA) or an antigen, e.g., that elicits an immune response (such as eliciting a potent and durable neutralizing antibody titer), such as mRNA encoding CMV glycoproteins gB and/or pentameric complex (PC)). In certain particular embodiments, the RNA is a small non-coding RNA, such as shRNA, ASO, tRNA, dsRNA, or a combination thereof.

In certain embodiments, the ADAS provided by the invention includes cargo comprising at least one component of a gene editing system. Components of a “gene editing system” include (or encode) proteins (or nucleic acids encoding said proteins) that can, with suitable associated nucleic acids, modify a DNA sequence of interest, such as a genomic DNA sequence, whether e.g., by insertion or deletion of a sequence of interest, or by altering the methylation state of a sequence of interest, as well as nucleic acids associated with the function of such proteins, e.g., guide RNAs. Exemplary gene editing systems include those based on a Cas system, such as Cas9, Cpf1 or other RNA-targeted systems with their companion RNA (e.g., sequence-complementary CRISPR guide RNA), as well as Zinc finger nucleases and TAL-effectors conjugated to nucleases.

Other embodiments of ADAS provided by the invention include DNA as the cargo, including as a plasmid, optionally wherein the DNA comprises a protein-coding sequence. Exemplary DNA cargo includes, in certain embodiments, a plasmid encoding an RNA sequence of interest (see examples above), e.g., which may be flanked on each side by an tRNA insert. Various DNA cargo are encompassed by the invention, including: ADAS producing (e.g., driving FTZ overexpression, genome degrading exonucleases); longevity plasmids (ATP synthase expressing, rhodopsin-expressing); those expressing stabilized non-coding RNA, tRNA, lncRNA; expressing secretion system tag proteins, NleE2 effector domain and localization tag; secretion systems T3/4SS, T5SS, T6SS; logic circuits, conditionally expressed secretion systems; and combinations thereof. In some embodiments, a logic circuit includes inducible expression or suppression cassettes, such as IPTG-inducible Plac promoter and the hrpR portion of the AND gate, and, for example, the heat-induced promoter pL (from phage lambda, which is usually suppressed by a thermolabile protein) and the hrpS portion of the AND gate. To engineer an OR gate, a sytem described by Rosado et al., PLoS Genetics, 2018 can be used. Briefly, a cis-repressed mRNA coding for RFP under a constitutive promoter can be used. The repression can then be removed in the presence of RAJ11 sRNA. Plasmids containing the IPTG-inducible promoter PLac and heat-induced promoter pL, both of which induce the expression of RAJ11 sRNA, can then be used. The output would then be RFP expression, which is seen in response to either input. These systems can be adapted to a variety of sensor-type functions.

ADAS provided by the invention, in some embodiments, include a transporter in the membrane. In certain embodiments, the transporter is specific for glucose, sodium, potassium, a metal ion, an anionic solute, a cationic solute, or water.

In some embodiments, the membrane of an ADAS provided by the invention comprises an enzyme. In particular embodiments, the enzyme is a protease, oxidoreductase, or a combination thereof. In some embodiments, the enzyme is chemically conjugated to the ADAS membrane, optionally via a linker to the exterior membrane.

E. ADAS Lacking Proteases, RNases, and/or LPS

In another aspect, the invention provides a composition comprising a plurality of ADAS (e.g., highly active ADAS), wherein the ADAS have a reduced protease level or activity relative to an ADAS produced from a wild-type parent bacterium. In some aspects, the ADAS is produced from a parent bacterium that has been modified to reduce or eliminate expression of at least one protease.

In another aspect, the invention provides a composition comprising a plurality of ADAS (e.g., highly active ADAS), wherein the ADAS have a reduced RNAse level or activity relative to an ADAS produced from a wild-type parent bacterium. In some aspects, the ADAS is produced from a parent bacterium that has been modified to reduce or eliminate expression of at least one RNAse. In some embodiments, the RNase is an endoribonuclease or an exoribonuclease.

In another aspect, the invention provides a composition comprising a plurality of ADAS, wherein the ADAS has been modified to have reduced lipopolysaccharide (LPS). In some embodiments, the modification is a mutation in Lipid A biosynthesis myristoyltransferase (msbB).

In certain embodiments, an ADAS provided by the invention lacks one or more metabolically non-essential proteins. A “metabolically non-essential protein” non-exhaustively includes: fimbriae, flagella, undesired secretion systems, transposases, effectors, phage elements, or their regulatory elements, such as flhC or OmpA. In some embodiments, an ADAS provided by the invention lacks one or more of an RNAse, a protease, or a combination thereof, and, in particular embodiments, lacks one or more endoribonucleases (such as RNAse A, RNAse h, RNAse III, RNAse L, RNAse PhyM) or exoribonucleases (such as RNAse R, RNAse PH, RNAse D); or serine, cysteine, threonine, aspartic, glutamic and metallo-proteases; or a combination of any of the foregoing.

F. ADAS Comprising a Targeting Moiety

In another embodiment, the invention provides a composition comprising a plurality of ADAS, wherein the ADAS comprises a targeting moiety. In some embodiments, the targeting moiety is a nanobody, a carbohydrate binding protein, or a tumor-targeting peptide. In some embodiments, the targeting moiety is an endogenous surface ligand of the parent cell (e.g., a surface ligand that is inherited by the ADAS). In other embodiments, the targeting moiety is an exogenous ligand (e.g., an exogenous tissue targeting ligand) that is added to the ADAS using any of the methods for modifying ADAS described herein. The targeting moiety may promote tissue-associated targeting of the ADAS to a tissue type or cell type.

In certain embodiments, the nanobody is nanobody directed to a tumor antigen, such as HER2, PSMA, or VEGF-R. In other embodiments, the carbohydrate binding protein is a lectin, e.g. Mannose Binding Lectin (MBL). In still other embodiments, the tumor-targeting peptide is an RGD motif or CendR peptide.

G. ADAS Derived from Commensal or Pathogenic Parent Strains

In another embodiment, the invention provides a composition comprising a plurality of ADAS (e.g., highly active ADAS), wherein the ADAS is derived from a parent bacterium that is a mammalian pathogen or a mammalian commensal bacterium. In some instances, the mammalian commensal bacterium is a Staphylococcus, Bifidobacterium, Micrococcus, Lactobacillus, or Actinomyces species or the mammalian pathogenic bacterium is enterohemorrhagic Escherichia coli (EHEC), Salmonella typhimurium, Shigella flexneri, Yersinia enterolitica, or Helicobacter pylori.

In another embodiment, the invention provides a composition comprising a plurality of ADAS (e.g., highly active ADAS), wherein the ADAS is derived from a parent bacterium that is a plant pathogen or a plant commensal bacterium. In some instances, the plant commensal bacterium is Bacillus subtilis or Psuedomonas putida or the plant pathogenic bacterium is a Xanthomonas species or Pseudomonas syringae.

H. ADAS Derived from Auxotrophic Parent Strains

In another embodiment, the invention provides a composition comprising a plurality of ADAS (e.g., highly active ADAS), wherein the ADAS is derived from an auxotrophic parent bacterium, i.e., a a parent bacterium that is unable to synthesize an organic compound required for growth. Such bacteria are able to grow only when the organic compound is provided.

I. ADAS Comprising Additional Moieties

An ADAS, in certain embodiments, includes a functional ATP synthase and, in some embodiments, a membrane embedded proton pump. ADAS can be derived from different sources including: a parental bacterial strain (“parental strain”) engineered or induced to produce genome-free enclosed membrane systems, a genome-excised bacterium, a bacterial cell preparation extract (e.g., by mechanical or other means), or a total synthesis, optionally including fractions of a bacterial cell preparation. In some embodiments, a highly active ADAS has an ATP synthase concentration of at least: 1 per 10000 nm², 1 per 5000 nm², 1 per 3500 nm², 1 per 1000 nm².

ADAS provided by the invention can include a variety of additional components, including, for example, photovoltaic pumps, retinals and retinal-producing cassettes, metabolic enzymes, targeting agents, cargo, bacterial secretion systems, and transporters, including combinations of the foregoing, including certain particular embodiments described, below. In certain embodiments, the ADAS lack other elements, such as metabolically non-essential genes and/or certain nucleases or proteases.

In certain embodiments, the an ADAS provided by the invention comprises an ATP synthase, optionally lacking a regulatory domain, such as lacking an epsilon domain. Deletion can be accomplished by a variety of means. In certain embodiments, the deletion in by inducible deletion of the native epsilon domain. In certain embodiments, deletion can be accomplished by flanking with LoxP sites and inducible Cre expression or CRISPR knockout, or be inducible (place on plasmid under a tTa tet transactivator in an ATP synthase knockout strain)

An ADAS, in some embodiments, can include a photovoltaic proton pump. In certain embodiments, the photovoltaic proton pump is a proteorhodopsin. In more particular embodiments, the proteorhodopsin comprises the amino acid sequence of proteorhodopsin from the uncultured marine bacterial clade SAR86, GenBank Accession: AAS73014.1. In other embodiments, the photovoltaic proton pump is a gloeobacter rhodopsin. In certain embodiments, the photovoltaic proton pump is a bacteriorhodopsin, deltarhodopsin, or halorhodopsin from Halobium salinarum Natronomonas pharaonis, Exiguobacterium sibiricum, Haloterrigena turkmenica, or Haloarcula marismortui.

In some embodiments, an ADAS provided by the invention further comprising retinal. In certain embodiments, an ADAS provided by the invention further comprises a retinal synthesizing protein (or protein system), or a nucleic acid encoding the same.

In certain embodiments, an ADAS provided by the invention further comprises one or more glycolysis pathway proteins. In some embodiments, the glycolysis pathway protein is a phosphofructokinase (Pfk-A), e.g., comprising the amino acid sequence of UniProt accession P0A796 or a functional fragment thereof. In other embodiments the glycolysis pathway protein is triosephosphate isomerase (tpi), e.g., comprising the amino acid sequence of UniProt accession P0A858, or a functional fragment thereof.

J. ADAS Compositions and Formulations

The present invention provides compositions or preparations that contain an ADAS provided by the invention, including, inter alia, a highly active ADAS preparation provided by the invention or an ADAS preparation wherein a plurality of individual ADAS lack a cell division topological specificity factor, e.g., lack a minE gene product, and optionally wherein the ADAS preparation is substantially free of viable cells. Collectively, these are “compositions provided by the invention” or “a composition provided by the invention”, or the like and can contain any ADAS provided by the invention and any combination of ADAS provided by the invention.

For example, in some embodiments, a composition provided by the invention contains at least about: 80, 81, 82, 83, 84, 85, 90, 95, 96, 97, 98, 99, 99.1, 99.2, 99.3, 99.4, 99.5, 99.6, 99.7, 99.8, 99.9%, or more ADAS that contain a bacterial secretion system. In particular embodiments, the bacterial secretion system is one of T3SS, T4SS, T3/4SS, or T6SS.

In some embodiments, a composition provided by the invention contains ADAS that contain a T3SS, where the ADAS comprise a mean T3SS membrane density greater than 1 in about: 40000, 35000, 30000, 25000, 19600, 15000, 10000, or 5000 nm². In certain particular embodiments, the ADAS is derived from a S. typhimurium or E. coli parental strain.

Certain embodiments of the compositions provided by the invention contain ADAS that contain a T3SS, where the ADAS comprise a mean T3SS membrane density greater than 1 in about: 300000, 250000, 200000, 150000, 100000, 50000, 20000, 10000, 5000 nm². In certain particular embodiments, the ADAS is derived from an Agrobacterium tumefaciens parental strain.

In another aspect, the invention provide a composition of ADAS, wherein at least about: 80, 81, 82, 83, 84, 85, 90, 95, 96, 97, 98, 99, 99.1, 99.2, 99.3, 99.4, 99.5, 99.6, 99.7, 99.8, 99.9%, or more of the ADAS contain a bacterial secretion system, including T3, T4, T3/4SS, T6SS, and optionally including one or more of: exogenous carbohydrates, phosphate producing synthases, light responsive proteins, import proteins, enzymes, functional cargo, organism-specific effectors, fusion proteins.

As will be readily apparent the compositions and preparations provided by the invention can contain any ADAS provided by the invention, such as highly active ADAS or ADAS that lack a minE gene product.

The compositions provided by the invention can be prepared in any suitable formulation. For example, the formulation can be suitable for IP, IV, IM, oral, topical (cream, gel, ointment, transdermal patch), aerosolized, or nebulized administration. In some embodiments, a formulation is a liquid formulation. In other embodiments, the formulation is a lyophilized formulation.

In some embodiments, an ADAS composition described herein comprises less than 100 colony-forming units (CFU/mL) of viable bacterial cells, e.g., less than 50 CFU/mL, less than 20 CFL/mL, less than 10 CFU/mL, less than 1 CFU/mL, or less than 0.1 CFU/mL of viable bacterial cells.

In some embodiments, the invention provides an ADAS composition wherein the ADAS are lyophilized and reconstituted, and wherein the reconstituted ADAS have an ATP concentration that is at least 90% of the ATP concentration of an ADAS that has not been lyophilized, e.g., at least 95%, 98%, or at least equal to the ATP concentration of an ADAS that has not been lyophilized.

In some embodiments, the invention provides an ADAS composition wherein the ADAS are stored, e.g., stored at 4° C., wherein after storage, the ADAS have an ATP concentration that is at least 90% of the ATP concentration of an ADAS that has not been stored, e.g., at least 95%, 98%, or at least equal to the ATP concentration of an ADAS that has not been stored. In some embodiments, the storage is for at least one day, at least one week, at least two weeks, at least three weeks, at least one month, at least two months, at least six months, or at least a year.

In some embodiments, ADAS may be preserved or otherwise in a “quiescent” state and then rapidly become activated.

In some embodiments, the ADAS composition is formulated for delivery to an animal, e.g., formulated for intraperitoneal, intravenous, intramuscular, oral, topical, aerosolized, or nebulized administration.

In some embodiments, the ADAS composition is formulated for delivery to a plant. In some aspects, the composition includes an adjuvant, e.g., a surfactant (e.g., a nonionic surfactant, a surfactant plus nitrogen source, an organo-silicone surfactant, or a high surfactant oil concentrate), a crop oil concentrate, a vegetable oil concentrate, a modified vegetable oil, a nitrogen source, a deposition (drift control) and/or retention agent (with or without ammonium sulfate and/or defoamer), a compatibility agent, a buffering agent and/or acidifier, a water conditioning agent, a basic blend, a spreader-sticker and/or extender, an adjuvant plus foliar fertilizer, an antifoam agent, a foam marker, a scent, or a tank cleaner and/or neutralizer. In some embodiments, the adjuvant is an adjuvant described in the Compendium of Herbicide Adjuvants (Young et al. (2016). Compendium of Herbicide Adjuvants (13th ed.), Purdue University).

In some embodiments, the ADAS composition is formulated for delivery to an invertebrate, (e.g., arthropod (e.g., insect or arachnid), nematode, protozoan, or annelid). In some embodiments, the ADAS composition is formulated for delivery to an insect.

In some embodiments, the composition is formulated as a liquid, a solid, an aerosol, a paste, a gel, or a gas composition.

K. ADAS Comprising an Enzyme

In one aspect, the invention features a composition comprising a plurality of ADAS, wherein the ADAS comprise an enzyme and wherein the enzyme alters a substrate to produce a target product. In some embodiments, the substrate is present in the ADAS and the target product is produced in the ADAS. In other embodiments, the substrate is present in a target cell or environment to which the ADAS is delivered. In some embodiments, the enzyme is diadenylate cyclase A, the substrate is ATP, and the target product is cyclic-di-AMP.

### III. Methods of Manufacturing ADAS

A. Making ADAS and Highly Active ADAS

In some aspects, the invention features a method for manufacturing a composition comprising a plurality of ADAS, the composition being substantially free of viable bacterial cells, the method comprising (a) making, providing, or obtaining a plurality of parent bacteria having a reduction in the level or activity of a cell division topological specificity factor; (b) exposing the parent bacteria to conditions allowing the formation of a minicell, thereby producing the highly active ADAS; and (c) separating the highly active ADAS from the parent bacteria, thereby producing a composition that is substantially free of viable bacterial cells.

Parent bacteria include any suitable bacterial species from which an ADAS may be generated (e.g., species that may be modified using methods described herein to produce ADAS). Table 1 provides a non-limiting list of suitable genera from which ADAS can be derived.

TABLE 1
Bacterial species for ADAS production
Gram negative bacteria
Escherichia
Acinetobacter
Agrobacterium
Anabaena
Anaplasma
Aquifex
Azoarcus
Azotobacter
Azospirillum
Bartonella
Bordetella
Bradyrhizobium
Brucella
Buchnera
Burkholderia
Candid atus
Chromobacterium
Crocosphaera
Coxiella
Dechloromonas
Desulfitobacterium
Desulfotalea
Erwinia
Francisella
Fusobacterium
Gloeobacter
Gluconobacter
Helicobacter
Legionella
Magnetospirillum
Mesorhizobium
Methylobacterium
Methylococcus
Neisseria
Nitrosomonas
Nostoc
Photobacterium
Photorhabdus
Phyllobacterium
Polaromonas
Prochlorococcus
Pseudomonas
Psychrobacter
Ralstonia
Rhizobium
Rickettsia
Rubrivivax
Salmonella
Shewanella
Shigella
Sinorhizobium
Synechococcus
Synechocystis
Thermosynechococcus
Thermotoga
Thermus
Thiobacillus
Trichodesmium
Vibrio
Wigglesworthia
Wolinella
Xanthomonas
Xylella
Yersinia
Gram positive bacteria
Bacillus
Clostridium
Deinococcus
Exiguobacterium
Geobacillus
Lactobacillus
Listeria
Moorella
Oceanobacillus
Symbiobacterium
Thermoanaerobacter

In some aspects, the invention features methods for manufacturing any of the ADAS compositions, e.g., highly active ADAS compositions, described in Section I herein. For example, provided herein are methods for making highly active ADAS; methods for making ADAS lacking a cell division topological specificity factor and, optionally, lacking a Z-ring inhibition protein (e.g., methods of making ADAS from ΔminCDE parent bacteria), and methods for making any of the ADAS mentioned herein, wherein the ADAS comprises a cargo.

In some embodiments, the ADAS (e.g., highly active ADAS) is made from a parental strain that is a plant bacterium, such as a plant commensal bacterium (e.g., Bacillus subtilis or Pseudomonas putida), a plant pathogen bacterium (e.g., Xanthomonas sp. or Psuedomonas syringae), or a bacterium that is capable of plant rhizosphere colonization and/or root nodulation, e.g., a Rhizobia bacterium.

In some embodiments, the ADAS (e.g., highly active ADAS) is made from a parental strain that is a symbiont of an invertebrate, e.g., a symbiont of an arthropod (e.g., insect or arachnid), nematode, protozoan, or annelid. In embodiments, the invertebrate is a pest or a pathogen of a plant or of an animal.

In some embodiments, the ADAS (e.g., highly active ADAS) is made from a parental strain that is capable of genetic transformation, e.g., Agrobacterium.

In some embodiments, the ADAS (e.g., highly active ADAS) is made from a parent strain that is a human bacterium, such as a commensal human bacterium (e.g., E. coli, Staphylococcus sp., Bifidobacterium sp., Micrococcus sp., Lactobacillus sp., or Actinomyces sp.) or a pathogenic human bacterium (e.g., Escherichia coli EHEC, Salmonella typhimurium, Shigella flexneri, Yersinia enterolitica, or Helicobacter pylon), or an extremophile.

In some embodiments, the ADAS and/or parent strain is a functionalized derivative of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

Parent bacteria may include functionalized derivatives of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

In some embodiments, an ADAS is derived from a parental strain engineered or induced to overexpress ATP synthase. In some more particular embodiments, the ATP synthase is heterologous to the parental strain. In certain particular embodiments, the parental strain is modified to express a functional FoFi ATP synthase.

In certain embodiments, an ADAS provided by the invention is obtained from a parental strain cultured under a condition selected from: applied voltage (e.g., 37 mV), non-atmospheric oxygen concentration (e.g., 1-5% O₂, 5-10% O₂, 10-15% O₂, 25-30% O₂), low pH (about: 4.5, 5.0, 5.5, 6.0, 6.5), or a combination thereof.

The highly active ADAS of any one of the preceding claims, which is made from an extremophile, including functionalized derivatives of any of the foregoing, for example including a functional cassettes, such as a functional cassette that induces the bacterium to do one or more of: secrete antimicrobials, digest plastic, secrete insecticides, survives extreme environments, make nanoparticles, integrate within other organisms, respond to the environment, and create reporter signals.

Owing to the diversity of bacterium, ADAS can be made with modified membranes, e.g., to improve the biodistribution of the ADAS upon administration to a target cell. In certain embodiments, the membrane is modified to be less immunogenic or immunostimulatory in plants or animals. For example, in certain embodiments, the ADAS is obtained from a parental strain, wherein the immunostimulatory capabilities of the parental strain are reduced or eliminated through post-production treatment with detergents, enzymes, or functionalized with PEG. In certain embodiments, the ADAS is made from a parental strain and the membrane is modified through knockout of LPS synthesis pathways in the parental strain, e.g., by knocking out msbB. In other particular embodiments, the ADAS is made from a parental strain that produces cell wall-deficient particles through exposure to hyperosmotic conditions.

In some embodiments, the methods include transforming a parental strain with an inducible DNAse system, such as the exol (NCBI GeneID: 946529) & sbcD (NCBI GeneID: 945049) nucleases, or the I-Ceul (e.g., Swissprot: P32761.1) nuclease. In more particular embodiments, the methods include using a single, double, triple, or quadruple auxotrophic strain and having the complementary genes on the plasmid encoding the inducible nucleases.

In some embodiments, the methods of the methods provided by the invention, the parental strain is cultured under a condition selected from: applied voltage (e.g., 37 mV), non-atmospheric oxygen concentration (e.g., 1-5% O₂, 5-10% O₂, 10-15% O₂, 25-30% O₂), low pH (4.5-6.5), or a combination thereof.

In certain embodiments, the methods of the methods provided by the invention, the parental strain lacks flagella and undesired secretion systems, optionally where the flagella and undesired secretion systems are removed using lambda red recombineering.

In some embodiments, the methods of provided by the invention, flagella control components are excised from the parental strain genome via, for example, insertion of a plasmid containing a CRISPR domain that is targeted towards flagella control genes, such as flhD and flhC.

In certain embodiments, the methods provided by the invention are for making a highly active ADAS, where an ADAS comprising a plasmid containing a rhodopsin-encoding gene is cultured in the presence of light. In more particular embodiments, the rhodopsin is proteorhodopsin from SAR86 uncultured bacteria, having the amino acid sequence of GenBank Accession: AAS73014.1, or a functional fragment thereof. In still more particular embodiments, the culture is supplemented with retinal. In other more particular embodiments, the rhodopsin is proteorhodopsin and the plasmid additionally contains genes synthesizing retinal (such a plasmid is the pACYC-RDS plasmid from Kim et al., Microb Cell Fact, 2012).

In certain particular embodiments, the parental strain contains a nucleic acid sequence encoding a nanobody that is then expressed on the membrane of the ADAS.

In some embodiments of the methods provided by the invention, the parental strain contains a nucleic acid sequence encoding one or more bacterial secretion system operons. Exemplary plasmids include the Salmonella SPI-1 T3SS, the Shigella flexneri T3SS, the Agro Ti plasmid, and the P. putida K1-T655 system.

In certain embodiments, the parental strain comprises a cargo. In some embodiments, the parent strain contains a nucleic acid sequence encoding a set of genes that synthesize a small molecule cargo.

### IV. Purification of ADAS and ADAS Compositions

In some embodiments of the methods and compositions provided herein, ADAS are purified from compositions (e.g., cultures) comprising viable bacteria, e.g., parental bacteria. For example, the invention features a method for manufacturing a composition comprising a plurality of ADAS, the composition being substantially free of viable bacterial cells, the method comprising (a) making, providing, or obtaining a plurality of parent bacteria having a reduction in the level or activity of a cell division topological specificity factor; (b) exposing the parent bacteria to conditions allowing the formation of a minicell, thereby producing the ADAS; and (c) separating the ADAS from the parent bacteria, thereby producing a composition that is substantially free of viable bacterial cells.

Purification separates ADAS from viable parent bacterial cells, which are larger and contain a genome. Separating the highly active ADAS from the parent bacteria can be performed using a number of methods, as described herein. Exemplary methods for purification described herein include centrifugation, selective outgrowth, and buffer exchange/concentration processes.

In some aspects, provide herein are ADAS compositions, and methods of comparing such compositions, wherein the compositions are substantially free of parent bacterial cells and/or viable bacterial cells, e.g., have no more than 500, e.g., 400, 300, 200, 150, or 100 or fewer than 50, fewer than 25, fewer than 10, fewer than 5, fewer than 1, fewer than 0.1colony-forming units (CFU) per mL. In some embodiments, an ADAS composition that is substantially free of parent bacterial cells may include no bacterial cells.

Auxotrophic parental strains can be used to make ADAS provided by the invention. As described in more detail below, such manufacturing methods are useful for purification of the ADAS. For example, following ADAS generation, parent bacterial cells may be removed by growth in media lacking the nutrient (for example, amino acid) necessary for viability of the parent bacterium. In some embodiments, an ADAS provided by the invention is derived from a parental strain auxotrophic for at least 1, 2, 3, 4, or more of: arginine (e.g., knockout in argA, such as strains JW2786-1 and NK5992), cysteine knockout in cysE (such as strains JW3582-2 and JM15), glutamine e.g., knockout in glnA (such as strains JW3841-1 and M5004), glycine e.g., knockout in glyA (such as strains JW2535-1 and ΔT2457), Histidine e.g., knockout in hisB (such as strains JW2004-1 and SB3930), isoleucine e.g., knockout in ilvA (such as strains JW3745-2 and AB1255), leucine e.g., knockout in leuB (such as strains JW5807-2 and CV514), lysine e.g., knockout in lysA (such as strains JW2806-1 and KL334), methionine e.g., knockout in metA (such as strains JW3973-1 and DL41), phenylalanine e.g., knockout in pheA (such as strains JW2580-1 and KA197), proline e.g., knockout in proA (such as strains JW0233-2 and NK5525), Serine e.g., knockout in serA (such as strains JW2880-1 and JC158), threonine e.g., knockout in thrC (such as strains JW0003-2 and Gif 41), tryptophan e.g., knockout in trpC (such as strains JW1254-2 and CAG18455), Tyrosine e.g., knockout in tyrA (such as strains JW2581-1 and N3087), Valine/Isoleucine/Leucine e.g., knockout in ilvd (such as strains JW5605-1 and CAG18431).

In certain embodiments, the methods include using a single, double, triple, or quadruple auxotrophic parental strain, optionally wherein said parental strain further includes a plasmid expressing a ftsZ.

### V. Methods of Using ADAS

A. Methods of Delivering an ADAS

In one aspect, the invention features a method for delivering an ADAS to a target cell, the method comprising (a) providing a composition comprising a plurality of ADAS; and (b) contacting the target cell with the composition of step (a).

In another aspect, the invention features a method for delivering an ADAS to a target cell, the method comprising: (a) providing a composition comprising a plurality of ADAS; and (b) contacting the target cell with the composition of step (a).

The target cell may be, e.g., an animal cell, a plant cell, or an insect cell.

B. Methods of Delivering a Cargo

In another aspect, the invention features a method for delivering a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)) to a target cell, the method comprising: (a) providing a composition comprising a plurality of achromosomal dynamic active systems (ADAS); and (b) contacting the target cell with the composition of step (a).

In another aspect, the invention features a method for delivering a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)) to a target cell, the method comprising: (a) providing a composition comprising a plurality of ADAS; and (b) contacting the target cell with the composition of step (a).

The target cell to which the cargo is delivered may be, e.g., an animal cell, a plant cell, or an insect cell.

C. Methods of Modulating a State of a Cell

In one aspect, the invention features a method of modulating a state of an animal cell, the method comprising: (a) providing a composition comprising a plurality of achromosomal dynamic active systems (ADAS); and (b) contacting the animal cell with the composition of step (a), whereby a state of the animal cell is modulated.

In another aspect, the invention features a method of modulating a state of a plant cell, the method comprising: (a) providing a composition comprising a plurality of achromosomal dynamic active systems (ADAS); and (b) contacting the plant cell with the composition of step (a), whereby a state of the plant cell is modulated.

In another aspect, the invention features a method of modulating a state of an insect cell, the method comprising: (a) providing a composition comprising a plurality of achromosomal dynamic active systems (ADAS); and (b) contacting the insect cell with the composition of step (a), whereby a state of the insect cell is modulated.

The modulating may be any observable change in the state (e.g., the transcriptome, proteome, epigenome, biological effect, or health or disease state) of the cell (e.g., an animal, plant, or insect cell) as measured using techniques and methods known in the art for such a measurement, e.g., methods to measure the level or expression of a protein, a transcript, an epigenetic mark, or to measure the increase or reduction of activity of a biological pathway. In some embodiments, modulating the state of the cell involves increasing a parameter (e.g., the level or expression of a protein, a transcript, or activity of a biological pathway) of the cell. In other embodiments, modulating the state of involves decreasing a parameter (e.g., the level or expression of a protein, a transcript, or activity of a biological pathway) of the cell.

D. Methods of Treating an Animal, a Plant, or an Insect

In some aspects, the invention features a method of treating an animal in need thereof, the method comprising (a) providing a composition comprising a plurality of achromosomal dynamic active systems (ADAS); and (b) contacting the animal with an effective amount of the composition of step (a), thereby treating the animal.

The animal in need of treatment may have a disease, e.g., a cancer. In some embodiments, the ADAS carries a chemotherapy cargo or an immunotherapy cargo.

In some aspects, the invention features a method of treating a plant in need thereof, the method comprising (a) providing a composition comprising a plurality of achromosomal dynamic active systems (ADAS); and (b) contacting the plant or a pest (e.g., an insect pest) thereof with an effective amount of the composition of step (a), thereby treating the plant.

In an additional aspect, the invention provides methods of modulating a target cell. The target cell can be any cell, including an animal cell (e.g., including humans and non-human animals, including farm or domestic animals, pests), a plant cell (including from a crop or a pest), a fungal cell, or a bacterial cell. The cell may be isolated, e.g., in vitro or, in other embodiments, within an organism, in vivo. These methods entail providing an ADAS provided by the invention or a composition provided by the invention with access to the target cell, in an effective amount. The access to the target cell may either be direct, e.g., where the target cell is modulated directly by the ADAS, such as by proximate secretion of some agent proximate to the target cell or injection of the agent into the target cell, or indirect. The indirect modulation of the target cell can be by targeting a different cell, for example, by modulating a cell adjacent to the target cell, which adjacent cell may be commensal or pathogenic to the target cell. The adjacent cell, like the target cell may be either in vitro or in vivo—i.e., in an organism, which may be commensal or pathogenic. These methods are collectively “methods of use provided by the invention” and the like. In a related aspect, the invention provides target uses of the ADAS and compositions provided by the invention, consonant with the methods of use provided by the invention.

For example, in some embodiments, the invention provides method of modulating a state of an animal cell, by providing an effective amount of an ADAS provided by the invention or composition provided by the invention access to the animal cell. In certain embodiments, the ADAS or composition is provided access to the animal cell in vivo, in an animal, such as a mammal, such as a human. In some embodiments, the animal cell is exposed to bacteria in a healthy animal. In more particular embodiments, the animal cell is lung epithelium, an immune cell, skin cell, oral epithelial cell, gut epithelial cell, reproductive tract epithelial cell, or urinary tract cell. In still more particular embodiments, the animal cell is a gut epithelial cell, such as a gut epithelial cell from a human subject with an inflammatory bowel disease, such as Crohn's disease or colitis. In yet more particular embodiments, the animal cell is a gut epithelial cell from a subject with an inflammatory bowel disease, and the ADAS comprises a bacterial secretion system and a cargo comprising an anti-inflammatory agent, e.g., an antibody or antibody fragment targeting tumor necrosis factor (TNF) (e.g., an anti-TNF antibody); an antibody or antibody fragment targeting IL-12 (e.g., an anti-IL-12 antibody); or an antibody or antibody fragment targeting IL-23 (e.g., an anti-IL-23 antibody).

In other embodiments the animal cell is exposed to bacteria in a diseased state. In certain embodiments, the animal cell is pathogenic, such as a tumor. In other embodiments, the animal cell is exposed to bacteria in a diseased state, such as a wound, an ulcer, a tumor, or an inflammatory disorder

In certain embodiments, the ADAS is derived from an animal commensal parental strain. In other embodiments, the ADAS is derived from animal pathogenic parental strain.

In certain particular embodiments, the animal cell is contacted to an effective amount of an ADAS comprising a T3/4SS or T6SS and a cargo, wherein the cargo is delivered into the animal cell. In some particular embodiments, the animal cell is provided access to an effective amount of an ADAS comprising a cargo and a secretion system, wherein the cargo is secreted extracellularly and contacts the animal cell.

In some embodiments, the state of the animal cell is modulated by providing an effective amount of an ADAS provided by the invention or a composition provided by the invention with access to a bacterial or fungal cell in the vicinity of the animal cell. That is, these methods entail indirectly modulating the state of the animal cell. In certain embodiments, the bacterial or fungal cell is pathogenic. In more particular embodiments, the fitness of the pathogenic bacterial or fungal cell is reduced. In other certain embodiments, the bacterial or fungal cell is commensal. In more particular embodiments, the fitness of the commensal bacterial or fungal cell is increased. In still more particular embodiments, the fitness of the commensal bacterial or fungal strain is increased via reduction in fitness of number of a competing bacteria or fungi, which may be neutral, commensal, or pathogenic.

In certain particular embodiments, the bacterial or fungal cell in the vicinity of the animal cell is contacted to an effective amount of ADAS comprising a T3/4SS or T6SS and a cargo, wherein the cargo is delivered into the bacterial or fungal cell. In other particular embodiments, the bacterial or fungal cell in the vicinity of the animal cell is provided access to an effective amount of ADAS secreting cargo extracellularly that contacts the bacterial or fungal cell.

In certain embodiments, the ADAS is derived from a parental strain that is a competitor of the bacterial or fungal cell. In other embodiments, the ADAS is derived from a from a parental strain that is mutualistic bacteria of the bacterial or fungal cell.

As will be appreciated, the various method of use provided by the invention that modulate the state of an animal cell can readily be adapted to corresponding methods for modulating the state of a plant, fungal, or bacterial cell. For illustrative purposes, methods for modulating the cell of a plant or fungal cell will be recited more particularly.

Accordingly, in a related aspect the invention provide methods of modulating a state of a plant or fungal cell by providing an effective amount of an ADAS provided by the invention or composition provided by the invention access to: a) the plant or fungal cell, b) an adjacent bacterial or adjacent fungal cell in the vicinity of the plant or fungal cell, or c) an invertebrate, (e.g., arthropod (e.g., insect or arachnid), nematode, protozoan, or annelid) cell in the vicinity of the plant or fungal cell.

In certain embodiments, the ADAS is provided access to the plant cell in planta, e.g., in a crop plant such as row crops, including corn, wheat, soybean, and rice, and vegetable crops including Solanaceae, such as tomatoes and peppers; cucurbits, such as melons and cucumbers; Brassicas, such as cabbages and broccoli; leafy greens, such as kale and lettuce; roots and tubers, such as potatoes and carrots; large seeded vegetables, such as beans and corn; and mushrooms. In some embodiments, the plant or fungal cell is exposed to bacteria in a healthy plant or fungus. In other embodiments, the plant or fungal cell is exposed to bacteria in a diseased state.

In certain embodiments, the plant or fungal cell is dividing, such as a meristem cell, or is pathogenic, such as a tumor. In some embodiments, the plant or fungal cell is exposed to bacteria in a diseased state, such as a wound, or wherein the plant or fungal cell is not part of a human foodstuff.

For certain embodiments the ADAS is derived from a commensal parental strain. In other embodiments, the ADAS is derived from a plant or fungal pathogenic parental strain.

In some embodiments, the ADAS comprises an T3/4SS or T6SS and a cargo, and the cargo is delivered into the plant or fungal cell. In other embodiments, the plant or fungal cell is provided access to an effective amount of an ADAS comprising a bacterial secretion system and a cargo, wherein the bacterial secretion system secretes the cargo extracellularly, thereby contacting the plant or fungal cell with the cargo.

In some embodiments, the methods entail providing an effective amount of an ADAS or composition access to the adjacent bacterial or adjacent fungal cell in the vicinity of the plant or fungal cell. In more particular embodiments, the adjacent bacterial or adjacent fungal cell is pathogenic, optionally wherein the fitness of the pathogenic adjacent bacterial or adjacent fungal cell is reduced. In other more particular embodiments, the adjacent bacterial or adjacent fungal cell is commensal, optionally wherein the fitness of the commensal adjacent bacterial or adjacent fungal cell is increased. In still more particular embodiments, the fitness is increased via reduction of a competing bacteria or competing fungi, which may be neutral, commensal, or pathogenic.

In some embodiments, the adjacent bacterial or adjacent fungal cell is contacted with an effective amount of ADAS comprising a T3/4SS or T6SS and a cargo, wherein the cargo is delivered into the adjacent bacterial or adjacent fungal cell.

In other embodiments, the adjacent bacterial or adjacent fungal cell is provided access to an effective amount of ADAS comprising a bacterial secretion system and a cargo, wherein the bacterial secretion system secretes the cargo extracellularly, thereby contacting the adjacent bacterial or adjacent fungal cell with the cargo.

In some embodiments, the ADAS is derived from a parental strain that is a competitor of the adjacent bacterial or adjacent fungal cells. In other embodiments, the ADAS is derived from a parental strain that is a mutualistic bacterium of the adjacent bacterial or adjacent fungal cell.

In certain embodiments, the methods include providing an effective amount of the ADAS or composition access to an invertebrate, (e.g., arthropod (e.g., insect or arachnid), nematode, protozoan, or annelid) cell in the vicinity of the plant or fungus. In more particular embodiments, the invertebrate is pathogenic. In still more particular embodiments, the fitness of the pathogenic invertebrate cell is reduced. In yet more particular embodiments, the fitness of the pathogenic invertebrate cell is reduced via modulation of symbiotes in the invertebrate cell. In other particular embodiments, the invertebrate is commensal. In more particular embodiments, the fitness of the commensal invertebrate cell is increased. In still more particular embodiments, the fitness is increased via reduction of a competing bacteria or fungi, which may be neutral, commensal, or pathogenic.

In yet another aspect, the invention provide methods of removing one or more undesirable materials from an environment comprising contacting the environment with an effective amount of an ADAS provided by the invention or composition provided by the invention, wherein the ADAS comprises one or more molecules (such as proteins, polymers, nanoparticles, binding agents, or a combination thereof) that take up, chelate, or degrade the one or more undesirable materials. “Environments” are defined as targets that are not cells, such as the ocean, soil, superfund sites, skin, ponds, the gut lumen, and food in a container.

In certain embodiments, the undesirable material includes a heavy metal, such as mercury, and the ADAS includes one or more molecules (such as proteins, polymers, nanoparticles, binding agents, or a combination thereof) that bind heavy metals, such as MerR for mercury. In some embodiments, the undesirable material includes a plastic, such as PET, and the ADAS includes one or more plastic degrading enzymes, such as PETase. In certain embodiments, the undesirable material comprises one or more small organic molecules and the ADAS comprise one or more enzymes capable of metabolizing said one or more small organic molecules.

E. RNA Delivery Methods

In another aspect, the invention provides a composition containing a bacterium or ADAS provided by the invention, wherein the bacterium or ADAS includes a T4SS, an RNA binding protein cargo, and an RNA cargo that is bound by the RNA binding protein and is suitable for delivery into a target cell through the T4SS. In certain embodiments, the RNA binding protein is a Cas9 fused to VirE2 and VirF, the RNA cargo is a guide RNA, and, optionally, the T4SS is the Ti system from Agrobacterium. In other embodiments, the RNA binding protein is p19 from Carnation Italian Ringspot Virus fused to VirE2 or VirF, the RNA cargo is an siRNA, and optionally wherein the T4SS is the Ti system from Agrobacterium.

In a related aspect, the invention provides methods of making these particular compositions, such methods entailing transfecting a plasmid containing the Cas9 fused to VirE2 and VirF and RNA cargo into an Agrobacterium cell.

In a further related aspect, the invention provides methods for delivering RNA to a plant cell or animal cell comprising contacting said plant cell or animal cell with a bacterium or ADAS, wherein the bacteria or ADAS comprises a T4SS, an RNA binding protein cargo, and an RNA cargo, wherein the RNA is delivered to the plant cell or animal cell. In more particular embodiments, the RNA-binding protein cargo is also delivered to the plant cell or animal cell.

### EXAMPLES

Table of Contents

| | |
|---|---|
| Example 1 | Methods for manufacturing ADAS with endogenous |
| | or heterologous secretion systems (T3-ADAS) |
| Example 2 | Synthesis of attenuated T3-ADAS via removal of |
| | endogenous effectors |
| Example 3 | Endogenous effectors delivered via T3-ADAS induce |
| | actin ruffling on mammalian host cells |
| Example 4 | Endogenous effectors delivered via T3-ADAS induce |
| | cytotoxicity in mammalian cells |
| Example 5 | Engineering heterologous T3-ADAS |
| Example 6 | Intracellular protein delivery to mammalian cells via |
| | heterologous T3-ADAS |
| Example 7 | T3-ADAS with enhanced expression of effectors |
| Example 8 | Translocating a T3-independent cargo via T3-ADAS |
| Example 9 | Translocating a T3-independent cargo via T3-ADAS |
| Example 10 | Methods for negative selection of viable parent |
| | contaminants |
| Example 11 | Characterization of T3-ADAS |

### Example 1: Methods for Manufacturing ADAS with Endogenous or Heterologous Secretion Systems (T3-ADAS)

Here, we describe two distinct approaches for generating bacterial ADAS capable of Type III secretion (T3-ADAS). The first approach utilizes bacterial strains that encode a native or “endogenous” type III secretion system (T3SS), i.e., a T3SS encoded by genes present in the wild-type parent bacterium. The second approach is characterized by the mobilization (e.g., via DNA capture methodologies or de novo DNA synthesis) of “heterologous” genetic elements encoding a T3SS into a bacterial strain that does not natively produce the T3SS (e.g., does not produce any T3SS). Endogenous or heterologous T3SS may be present in ADAS produced by any method, and ADAS comprising a T3SS may be referred to as T3 ADAS.

T3 ADAS may be manufactured using any of the methods for ADAS production described herein. Such methods include disruption of the regulation of parent cell partitioning functions, e.g., disruption of the min operon or over-expression of the septum machinery component FtsZ.

Additionally, the abundance of either endogenous or heterologous T3SSs within ADAS can be increased, e.g., via modulation of transcriptional regulatory networks in ADAS-producing bacteria. Most T3SSs have associated transcriptional regulators, including one or more positive regulators, which promote T3SS expression, and one or more negative regulators, which antagonize T3SS expression, typically via inhibition of the positive regulators. Overexpression of positive regulators and/or deletion of negative regulators is sufficient to increase T3SS expression in bacteria and the ADAS these bacteria produce.

Here, we describe genetic engineering, microbial culture, and bioprocessing procedures utilized to generate T3 ADAS comprising endogenous and heterologous T3SSs. Exemplary T3 ADAS comprising endogenous T3SSs were produced by engineering parent strains of Salmonella enterica and Vibrio parahaemolyticus to produce bacterial ADAS and overexpress their native T3SSs. Exemplary T3 ADAS comprising heterologous T3SSs were produced by engineering multiple ADAS-producing strains of Escherichia coli to overexpress a heterologous T3SS captured from the virulence plasmid of Shigella flexneri.

A. Engineering ADAS Production in a Bacterial Chassis Having an Endogenous Type 3 Secretion System

In some cases, ADAS are made from a gram-negative bacterial species that naturally encodes and expresses a T3SS, thereby generating T3 ADAS comprising an endogenous T3SS. In this example, T3 ADAS were generated from Salmonella enterica and Vibrio parahaemolyticus, two exemplary bacterial strains having endogenous T3SS. Disruption of the minCDE locus was used as an exemplary method for producing ADAS.

Distinct molecular genetic procedures were performed to disrupt the minCDE locus in Salmonella enterica (LT2) and Vibrio parahaemolyticus (ATCC 17802), cell lines obtained from the American Type Culture Collection (ATCC).

For S. enterica, the minCDE locus was replaced with either a chloramphenicol- or kanamycin-resistance cassette using standard lambda red recombination protocols (Datsenko and Wanner, PNAS, 97(12): 6640-6645, 2000). Briefly, primers were designed with a 5′ region, which introduces 40 base pairs of homology with the genomic region flanking the minCDE locus, and a 3′ region, which introduces additional regions of homology with the plasmids pKD3 or pKD4 of the Lambda-RED system. These primers were used to amplify the drug resistance cassettes of either pKD3 or pKD4, and the resulting PCR product was electroporated into a S. enterica strain bearing pKD46, a plasmid encoding the phage-derived Lambda-RED homologous recombination system, according to the methods of Datsenko and Wanner, PNAS, 97(12): 6640-6645, 2000. Successful transformants, which underwent a recombination event that replaced the chromosomal minCDE locus with the drug-resistance cassette, were selected on LB agar with chloramphenicol 35 μg/mL or kanamycin 50 μg/mL. The resulting colonies are confirmed to have the genetic disruption using standard allele-specific PCR.

ADAS-producing strains of V. parahaemolyticus were generated via sequential rounds of allelic exchange cloning in a spontaneous streptomycin-resistant mutant of V. parahaemolyticus ATCC17802 (MACH077) (Milton et al., Journal of Bacteriology, 178(5): 1310-1319, 1996). The first allelic exchange step used PRO231, a pDM4 derived plasmid, to replace the chromosomal minCDE locus with a constitutively expressed Kanamycin-resistance cassette, generating MACH462 (ΔminCDE::kanR) (Table 2). The second allelic exchange step used PRO200 (Table 3), a different pDM4-derived plasmid, to delete the Kanamycin-resistance cassette of MACH462 (Table 2), generating MACH625 (ΔminCDE)
(Table 2). Allelic exchange vectors were created by amplifying template sequences (either ATCC17802 gDNA or a plasmid-encoded kanR cassette) with specific primers containing 20-40 base pair overhangs suitable for isothermal assembly. PCR products were purified and mixed at equimolar ratios and cloned into Smal-digested pDM4 via isothermal assembly. The resulting vectors were propagated in Pir1 E coli, transformed into SM10lambdaPir E. coli (MACH260), and delivered into V. parahaemolyticus via conjugation.

TABLE 2
Strains

| Name | Description | Genotype | Alias | Reference |
|---|---|---|---|---|
| MACH009 | BW25113 | Δ(araD- | CGSC 7636 | |
| | | araB)567, ΔlacZ4787(::rrnB- | | |
| | | 3), λ-, rph-1, Δ(rhaD- | | |
| | | rhaB)568, hsdR514 | | |
| MACH060 | MACH009 | BW25113 ΔminCDE::CamR | | This work |
| | ΔminCDE::CamR | | | |
| MACHO65 | MG1655 | F-, λ-, rph-1 | CGSC 7740 | |
| MACH077 | Vibrio | Smr1 | ATCC 17802 | |
| | parahaemolyticus, | | | |
| | Smr1 | | | |
| MACH 124 | MACH060 pGFP | BW25113 ΔminCDE::CamR | | |
| | | pGFP | | |
| MACH185 | Salmanella | Salmonella LT2 | | |
| | typhimerium + minCDE | minCDE::CamR | | |
| MACH199 | Vibrio | Vibrio parahaemolyticus | ATCC 17802 | |
| | parahaemolyticus | EB101 pFtsZ | | |
| | pFtsZ | | | |
| MACH200 | MACH065 | MG1655 ΔminCDE::CamR | | |
| | ΔminCDE::CamR | | | |
| MACH260 | SM10 lambda pir with | thi thr leu tonA lacY supE | | |
| | pDM4 | recA::RP4-2-Tc::Mu Km λpir | | |
| MACH269 | SalTy ΔminCDE pHilA- | Salmonella typhimurium LT2 | ATCC 23564 | |
| | KanR | ΔminCDE::CamR pHilA-KanR | | |
| MACH270 | SalTy ΔminCDE pHilA- | Salmonella typhimurium LT2 | | |
| | AmpR | ΔminCDE::CamR pHilA-AmpR | | |
| MACH320 | MACH060 pT3SS | BW25113 ΔminCDE::CamR | | |
| | pVirB | pT3SS pVirB | | |
| MACH434 | MACH060 pOspB- | BW25113 ΔminCDE::CamR | | |
| | TEM1 | pOspB-TEM1 | | |
| MACH435 | MACH060 pT3SS | BW25113 ΔminCDE::CamR | | |
| | pVirB pOspB-TEM1 | pT3SS pVirB pOspB-TEM1 | | |
| MACH462 | Vibrio | Vibrio parahaemolyticus with a | | |
| | parahaemolyticus | KanR cassette knocked in at | | |
| | (ATCC 17802) Smr1 | the minCDE locus | | |
| | ΔminCDE::KanR | | | |
| MACH607 | DH10B_Shigella | | | Du et al., |
| | T3SSΔeffectors | | | PNAS, |
| | | | | 113(17), |
| | | | | 4794-4799, |
| | | | | 2016 |
| MACH625 | Vp SmR1 ΔminCDE | ΔminCDE mutant created by | | |
| | | resolving the ΔminCDE::KanR | | |
| | | allele | | |
| MACH704 | MACH060 pT3SS | BW25113 ΔminCDE::CamR | | |
| | pVirB PRO292 (NleC) | pT3SS pVirB PRO292 (NleC) | | |
| MACH707 | MACH060 pT3SS | BW25113 ΔminCDE::CamR | | |
| | pVirB PRO295 (OspF) | pT3SS pVirB PRO295 (OspF) | | |
| MACH739 | Vp SmR1 ΔminCDE + | ΔminCDE mutant of Vp | | |
| | pMB1 _KanR_pTet_ | transformed with tet inducible | | |
| | exsA | exsA (transcriptional activator | | |
| | | of T3SS1) | | |
| MACH778 | MACH60 + PRO292 | MACH60 + PRO292 (NleC) | | |
| | (NleC) | | | |
| MACH869 | Deletion of Vp T3SS1 | Vp SmR1 ΔminCDE ΔT3Es | | |
| | effector locus via | | | |
| | conjugation | | | |
| MACH823 | MACH009 (BW25113) | MACH009 (BW25113) P1 | | |
| | P1 transduced with | transduced with int_SER4 | | |
| | int_SER4 (integrated | (integrated T3SS lacking 4 | | |
| | T3SS lacking 4 | effectors) from MACH607 | | |
| | effectors) from | | | |
| | MACH607 | | | |
| MACH871 | Vp SmR1 ΔminCDE + | exsA expression vector | | |
| | pTet_exsA_pSC 101_ | transformed into T3E+ | | |
| | KanR | minCDE Vp | | |
| MACH872 | Vp SmR1 ΔminCDE | exsA expression vector | | |
| | ΔT3Es + | transformed into ΔT3Es | | |
| | pTet_exsA_pSC 101_ | minCDE Vp | | |
| | KanR | | | |
| MACH875 | BW25113_Shigella | Chromosome: | | |
| | T3SSΔeffectors_ | BW25113_Shigella | | |
| | ΔminCDE::CamR | T3SSΔeffectors (KanR) for | | |
| | | integrated | | |
| | | minT3SS_Δ4effectors and | | |
| | | ΔminCDE::CamR (CamR) | | |
| MACH953 | SM10 + pDM4_ΔexsD | Donor strain for Vp allelic | | |
| | | exchange vector to delete | | |
| | | exsD | | |
| MACH976 | BW25113_Shigella | Chromosome: | | |
| | T3SSΔeffectors_ | BW25113_Shigella | | |
| | ΔminCDE::CamR + | T3SSΔeffectors (KanR) for | | |
| | PRO101 (VirB) | integrated | | |
| | | minT3SS_Δ4effectors and | | |
| | | ΔminCDE::CamR (CamR) | | |
| | | Plasmid: IPTG-inducible VirB | | |
| | | (SpectR) | | |
| MACH4001 | SalTy ΔminCDE ΔinvA | Salmonella LT2 | | |
| | | minCDE::CamR, invA::KanR | | |
| MACH4002 | Vp SmR1 ΔminCDE | Vp SmR1 ΔminCDE ΔesxD | | |
| | ΔesxD | | | |
| MACH4003 | Vp SmR1 ΔminCDE + | exsA expression vector | | |
| | pCloDF_KanR TetR- | transformed into T3E + | | |
| | pTetA_exsA | minCDE Vp | | |
| MACH4004 | V. parahaemolyticus | V. parahaemolyticus ΔminCDE | | |
| | ΔminCDE ΔT3Es | ΔT3Es ΔVPA0450-0451 | | |
| | ΔVPA0450-0451 | | | |

TABLE 3
Plasmids

| Name | Description | Sequence | Reference |
|---|---|---|---|
| pGFP | pMB1 KanR TetR-PtetA-sfGFP | SEQ ID NO: 1 | This work |
| pHilA-CloDF | CloDF13 AmpR TetR-PtetA-HilA | SEQ ID NO: 2 | This work |
| pHilA-pMB1 | pMB1 AmpR TetR-PtetA-HilA | SEQ ID NO: 3 | This work |
| pOspB-TEM1 | ColE1 AmpR Plac-OspB-TEM1 | SEQ ID NO: 4 | Reeves et. al., |
| | | | ACS Syn Bio, |
| | | | 4: 644-654, |
| | | | 2015 |
| pT3SS | RK2 KanR Shigella flexneri minimum | SEQ ID NO: 5 | Mou et |
| | type 3 secretion system | | al., PNAS, |
| | | | 115(25): 6452- |
| | | | 6457, 2018 |
| pVirB | pSC101 SpecR Plac-VirB | SEQ ID NO: 6 | Reeves et. al., |
| | | | ACS Syn Bio, |
| | | | 4: 644-654, |
| | | | 2015 |
| pDM4 | A conjugation vector for doing allelic | SEQ ID NO: 7 | Milton et al., |
| | exchange. Uses R6K origin as suicide | | Journal of |
| | vector (must be propogated in pir+ | | Bacteriology, |
| | strain) that in SM10/S17 E. coli | | 178(5): 1310- |
| | strains is mobilized via conjugation. | | 1319, 1996 |
| | With flanking regions of homology | | |
| | (homology arms) selection is | | |
| | performed using CamR resistance | | |
| | and then counterselected for double- | | |
| | cross over events using SacB. | | |
| Ptet_blaMSecSigtrap_ | Reporter plasmid for monitoring | SEQ ID NO: 8 | |
| AmpR_pMB1 | translocation into mammalian cells | | |
| | loaded with the CCF2/AM or | | |
| | CCF4/AM dye. Contains Bbsl sites to | | |
| | T2S assemble putative secretion tags | | |
| | in front of a TEM1 Beta-lactamase. | | |
| | When induced by ATC, if the TEM1 | | |
| | protein is translocated into a cell | | |
| | loaded with the reporter dye, the | | |
| | TEM1 activity creates a FRET-shift in | | |
| | the dye from Green to Blue. | | |
| Ptet_blaMSecSigtrap_ | Ptetwith a beta-lactamase (blaM; | SEQ ID NO: 9 | |
| CamR_pMB1 | TEM1) without a secretion signal. | | |
| | Secretion signal and RBS can be | | |
| | cloned into the Bbsl sites to use as a | | |
| | reporter plasmid of translocation into | | |
| | cells (e.g., T3SS) using the BLAzer | | |
| | reporter assay or extracellular | | |
| | secretion (e.g., T1SS, T2SS). | | |
| PRO200 | pDM4_ΔminCDE, allelic exchange | SEQ ID NO: 10 | |
| | vector bearing regions homologous to | | |
| | the minCDE locus in V. | | |
| | parahaemolyticus | | |
| pDM4_ΔminCDE::kanR | Allelic Exchange vector bearing | SEQ ID NO: 11 | |
| | regions homologous to the minCDE | | |
| | locus in V. parahaemolyticus with a | | |
| | KanR cassetts | | |
| PRO292 | ATC inducible expression of NleC- | SEQ ID NO: 12 | Zurawski et al., |
| | FLAG from EPEC | | Molecular |
| | | | Microbiology, |
| | | | 71(2): 350-368, |
| | | | 2009 |
| PRO295 | ATC inducible expression of Shigella | SEQ ID NO: 13 | Zurawski et al., |
| | ospF protein | | Molecular |
| | | | Microbiology, |
| | | | 71(2): 350-368, |
| | | | 2009 |
| pTet_exsA_pMB1 _ | exsA cloned into the Bsal site of | SEQ ID NO: 14 | |
| KanR | pTet_pMB1_Kan | | |
| Ptet_CloDF_Carb_dsA | Ptet, CloDF ori, Carb Resistance plus | SEQ ID NO: 15 | |
| ZR030-SopE-FLAG | Salmonella SopE-FLAG effector | | |
| PRO055 | Expression vector containing the TetR | SEQ ID NO: 16 | |
| | repressor and Ptet (TetA) promoter | | |
| | with Bsal cloning sites for inserting | | |
| | translational unit for expression. The | | |
| | PtetA promoter is then induced by the | | |
| | addition of anhydrotetracycline (ATC). | | |
| pOspC3(50aa)-TEM | shigella-derived secretion signal | SEQ ID NO: 17 | |
| | before the beta lactamase for the | | |
| | FRET translocation assay. | | |
| pSopE(104aa)-TEM | salmonella-derived secretion signal | SEQ ID NO: 18 | |
| | before the beta lactamase for the | | |
| | FRET translocation assay. | | |
| pExoS(54aa)-TEM | pseudomonas-derived secretion | SEQ ID NO: 19 | |
| | signal before the beta lactamase for | | |
| | the FRET translocation assay. | | |
| pYopE(50aa)-TEM | yersinia-derived secretion signal | SEQ ID NO: 20 | |
| | before the beta lactamase for the | | |
| | FRET translocation assay. | | |
| PRO231 | Allelic Exchange vector bearing | SEQ ID NO: 50 | |
| | regions homologous to the minCDE | | |
| | locus in V. parahaemolyticus with a | | |
| | CamR cassette | | |
| PRO330 | pMB1_KanR TetR-PletA-exsA | SEQ ID NO: 51 | |
| PRO369 | pCloDF_KanR TetR-pTetA_exsA | SEQ ID NO: 52 | |
| PRO370 | pSC101_KanR TetR-pTetA_exsA | SEQ ID NO: 53 | |
| PRO364 | Derivative of pDM4, Allelic exchange | SEQ ID NO: 54 | |
| | vector for deletion of Vp T3SS1 | | |
| | effector locus | | |
| PRO443 | Derivative of pDM4, allelic exchange | n/a | |
| | vector for_ΔVPA0450_0451 | | |

TABLE 4
Primers

| Primer Name | Description | Sequence |
|---|---|---|
| oAF75 | KO MinCDE FWD | SEQ ID NO: 21 |
| oAF76 | KO MinCDE REV | SEQ ID NO: 22 |
| oAF77 | KO MinC FWD | SEQ ID NO: 23 |
| oAF78 | KO MinC REV | SEQ ID NO: 24 |
| oAF79 | KO MinD FWD | SEQ ID NO: 25 |
| oAF80 | KO MinD REV | SEQ ID NO: 26 |
| oAF167 | KO SalTy MinCDE FWD | SEQ ID NO: 27 |
| oAF168 | KO SalTy MinCDE REV | SEQ ID NO: 28 |

B. Enhancing T3SS Expression in ADAS-Producing Strains T3SS expression is typically a tightly regulated process; however, the abundance of T3SSs on the surface of a bacterium or ADAS can be increased through the induction of a T3-specific transcriptional activator. For each T3 chassis organism, the corresponding transcriptional regulator is listed in Table 2. To control the expression of T3SSs, transcriptional regulators are cloned into expression constructs and placed under control of a constitutive or an inducible promoter. Expression constructs are then introduced into the corresponding T3-ADAS producing bacterial chassis either on an episomal plasmid or by site-specific integration into the host chromosome.

This approach was used to increase T3SS expression in both Salmonella and Vibrio T3 ADAS producing strains. In Salmonella, the SPI-1 encoded T3SS is regulated by the HilA transcriptional activator (Table 5). HilA was cloned into an anhydrotetracycline-inducible plasmid containing a CloDF13 origin of replication and a carbenicillin resistance gene to generate pHi1A-CloDF (Table 3) and introduced into the ΔminCDE Salmonella strain to create MACH269 (Table 2). T3-ADAS are synthesized from these strains using the methods described in Example 1.

For Vibrio, ExsA expression vectors were generated by amplifying the chromosomal allele with specific primers containing T2S restriction sites and cloned using T2S assembly into a variety of expression vectors, with distinct origins of replication, downstream of the an hydrotetracycline-inducible promoter. The resulting vectors (PRO330, PRO369, and PRO370) (Table 3) were transformed into Vp ΔminCDE via electroporation to generate MACH739, MACH4003, and MACH871 (Table 2), respectively. Increased expression of T3 operons was confirmed through T3 gene-specific qRT-PCR following growth in media prepared with or without inducer.

Alternatively, the promoter region of the T3 activator protein can be replaced directly with an inducible or constitutive promoter using KIKO (Sabri et al., Microbial Cell Factories, 12: Article No: 60, 2013), lambda red recombination methods, or allelic exchange protocols, as described above.

TABLE 5
T3SS activators listed by organism

| | Positive | Negative |
|---|---|---|
| T3 Bacterial chassis | Regulator | Regulator |
| Salmonella SPI-1 | HilA | |
| Shigella | VirB or VirF | H-NS |
| Vibrio | ExsA | ExsD |
| Yersinia | LcrF (VirF) | |
| E. coli | Ler | |
| Pseudomonas | ExsA | ExsD |

C. Enhancing T3SS Expression in ADAS Producing Strains Via Removal of T3 Negative Regulators (Repressors)

Enhanced expression of T3SS genes is also accomplished by removing factors involved in the negative regulation of T3 operons. In this example, T3SS-containing ADAS derived from Vibrio parahamemolyticus are used as model ADAS, and the chromosomal T3SS anti-sigma factor exsD is used as a model negative regulator of T3SS genes.

Deletion of exsD is generated via sequential rounds of allelic exchange cloning to generate MACH4002 (ΔminCDE ΔexsD; Table 5). Allelic exchange vectors are generated by amplifying template sequences (either V. parahamemolyticus gDNA or plasmid-encoded KanR cassette) with specific primers containing 20-40 bp overhangs suitable for isothermal assembly. PCR products are purified and mixed at equimolar ratios and cloned into Smal-digested pDM4 (Milton et al., Journal of Bacteriology, 178(5): 1310-1319, 1996) via isothermal assembly. The resulting vectors are propagated in Pir1 E. coli, transformed into SM10lambdaPir E. coli, and delivered into V. parahamemolyticus via conjugation. Increased expression of T3 operons in the exsD knockout V. parahamemolyticus line is confirmed by comparing T3 gene expression levels to wild-type bacteria using qRT-PCR.

### Example 2: Synthesis of Attenuated T3-ADAS Via Removal of Endogenous Effectors

Many of the natural substrates of T3 secretion systems (effectors) can modulate a variety of normal cellular processes upon intracellular delivery to mammalian cells. These processes include, but are not limited to the remodeling of cytoskeletal components, activation or inhibition of inflammatory pathways, and induction of cytotoxic responses in the host. Exemplary effectors and functional classes are shown in Table 6. This example describes strategies for removing some or all these effector proteins from bacterial species that naturally encode them and generating T3-ADAS therefrom, thus generating attenuated (minimal) T3-ADAS.

TABLE 6
Effectors and functional classes

| | | | Modulation of | |
|---|---|---|---|---|
| | | Cytoskeletal | Inflammatory | Additional |
| Organism | Cytotoxic | remodeling | Pathways | Effectors |
| Yersinia | | YopE, YopH, | YopJ, YspK, | |
| enterocolytica | | YopO, YopT | YopM | |
| Pseudomonas | ExoU, | ExoT, ExoS | | |
| aeriginosa | ExoY | | | |
| Salmonella | | SspH2, SteC, | AvrA, SspH1, | SifA, SIrP, |
| enterocolitica | | SrfH, SpvB, | SpvC, GogB, | SseF, SseG, |
| | | SipA, SptP, | SseK1, SseK2, | PipB2, |
| | | SopE2 | SseK3, GtgA, | SopD2, |
| | | | SpvD, SopE, | SSeJ, GtgE, |
| | | | SopA, SopB | PipB, SifB, |
| | | | | SteA, SteB, |
| | | | | SteD, SteE, |
| | | | | CigR, |
| | | | | SopD, SpiC |
| Shigella | | IpgB1, IpgB2, | IpaH7.8, | OspE2 |
| flexneri | | IpgD, VirA, | OspC1, OspB, | |
| | | IcsB, IpaA | OspF, OspG, | |
| | | | IpaH9.8, | |
| | | | OspZ, IpaH4.5 | |
| Vibrio | VPA450, | VopC, VopL, | VopP, VopZ, | |
| parahaemo- | VopT, | VopV | VopS, VopQ | |
| lyticus | VPA1380 | | | |
| Escherichia | Cif | EspH, Map, | NleB, NleC, | EspF, NleG, |
| coli | | Tir, EspG, | NleE, NleH1, | EspL, EspO |
| | | EspG2, EspJ, | NleD, NleF, | |
| | | NleL, EspZ, | NleA | |
| | | EspW, EspT, | | |
| | | EspM | | |

A. Removing T3Es from the V. parahaemolyticus Genome.

The four type III effectors (T3Es) known to be translocated through V. parahaemolyticus' T3SS are deleted via sequential rounds of allelic exchange cloning as described above. First, a large swath of the T3SS pathogenicity island containing 3 T3Es and their associated chaperones was deleted from MACH625 using the pDM4-derived allelic exchange vector pDM4_ΔT3Es (PRO364) to generate V. parahaemolyticus ΔminCDE ΔT3Es (MACH869). Then, the allelic exchange procedure was repeated using the pDM4-derived allelic exchange vector pDM4_ΔVPA0450-0451 (PRO443) to delete the remaining effector (VPA0450) and its associated chaperone (VPA0451) from the genome of the MACH869 to generate V. parahaemolyticus ΔminCDE ΔT3Es ΔVPA0450-0451 (MACH4004).

B. Attenuation of Endogenous T3-ADAS Via Recombination

T3 effectors are present within the operons encoding the T3SS structural machinery, but many are encoded in separate or individual transcriptional units located throughout the chromosome, on a virulence plasmid, or clustered in distinct pathogenicity islands. In this example, Yersinia enterocolitica and enteropathogenic E. coli (EPEC) are used as model parent bacteria.

In the case of the virulence plasmid of Y. enterocolitica (Derbise et al., FEMS Immunology & Medical Microbiology, 38(2): 113-116, 2003) and both LEE and non-LEE encoded effectors of EPEC (Cepeda-Molero et al., PLoS Pathogens, 13(10): e1006706, 2017), several effectors are removed in one step using standard recombination-based approaches, as described in Example 1. Sequential rounds of effector deletions can be pursued to target multiple loci in these bacterial chassis.

C. Attenuation of Multiple Effectors Via Removal of a Common Transcriptional Activator

In some cases, a suite of effectors are transcriptionally controlled by the same transcriptional activator. Thus, an alternative approach to individually removing effector proteins is to knock out a common regulator protein. In this example, Shigella flexneri is used as a model organism and mxiE is used as a model transcriptional activator.

In S. flexneri, the mxiE transcriptional activator induces expression of about half (˜16-20) of Shigella's T3 substrates (Kane et al., Journal of Bacteriology, 184(16), 4409-4419). mxiE is removed from a chassis strain (e.g., an ADAS-producing strain) using standard recombination-based approaches, as described in Example 1.

### Example 3: Endogenous Effectors Delivered Via T3-ADAS Induce Actin Ruffling on Mammalian Host Cells

T3SSs have been shown to modulate the cytoskeletal machinery of a variety of cell types (Navarro-Garcia et al., BioMed Research International, 2013: Article ID 374395, 2013. This example demonstrates that ADAS expressing a T3SS are capable of delivering effectors into human cells that induce changes to cytoskeleton to cause actin ruffling, a classic mechanism used by bacteria to invade mammalian cells. ADAS derived from Salmonella typhimurium were used as model ADAS, and human dermal fibroblast (HDFn) cells were used as model target cells. This model embodiment may be applied to modulating a cytoskeletal state of any number of animal and plant target organisms, e.g., via ADAS delivery of an endogenous effector through an endogenous T3SS.

A. Purification of SalTy T3-ADAS

Salmonella typhimurium (SalTy) T3-ADAS were prepared from MACH269 (Table 2) using methods described in Example 2, with several modifications to the growth and purification conditions. Anhydrotetracycline was added to the 250 mL culture to a final concentration of 50 ng/mL to induce over-expression of HilA and the SPI-1 T3SS apparatus. Purification steps in Example 9 were modified to improve parent cell removal by performing an initial 20-minute centrifugation at 2000×g and 4° C. Supernatant was decanted into fresh centrifuge tubes, and the 20-minute centrifugation at 2000×g and 4° C. was repeated. To remove remaining parental bacteria, the supernatant containing SalTy ADAS was transferred into a 1 L shake flask, to which ciprofloxacin (10 ug/mL) was added, followed by a 1 hour outgrowth at 37° C. in a 250 rpm shaking incubator. The remaining purification steps are performed in accordance with Example 2. At the final ADAS resuspension step, T3SS-expressing SalTy ADAS were resuspended in PBS and quantified using a Spectradyne® nCS1™ Nanoparticle Analyzer.

B. Actin Ruffling Using SalTy ADAS and Fibroblast Cells

To assess whether SalTy ADAS can mediate a change in actin dynamics on human fibroblast cells, SalTy ADAS were incubated with HDFn cells and stained for actin to visualize the presence of actin ruffles using fluorescence microscopy.

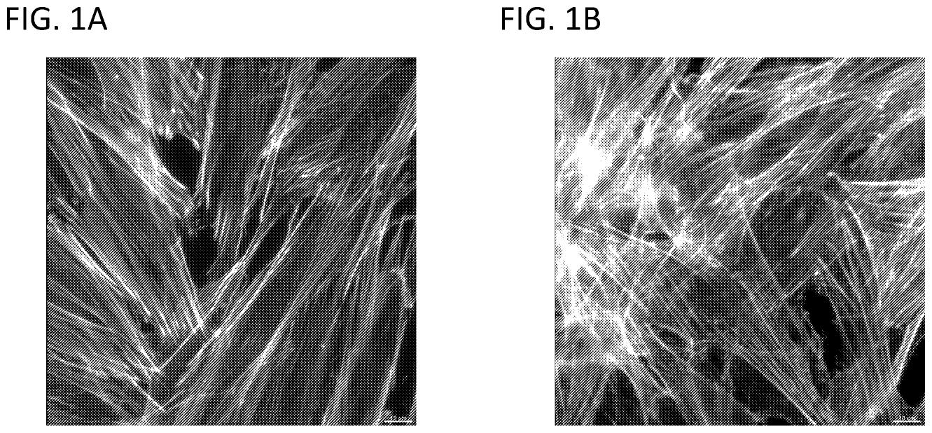

First HDFn cells were grown according to ATCC recommendations. For this assay, the cells were seeded in IBIDI® 35 mm dishes with glass coverslip bottoms. The cells were allowed to adhere for at least 24 hours prior to the assay. MACH269 SalTy T3-ADAS and MACH060 ADAS were added to separate dishes of HDFn cells at a ratio of 1000:1 (ADAS: HDFn) in cell media (DMEM plus 1% fetal bovine serum) for 40 minutes at 37° C. in an incubator with a 5% CO₂ environment. After 40 minutes, the cell media containing ADAS was aspirated and the cells were washed 2× with clean PBS. Then, the cells were fixed using 4% paraformaldehyde for 35 minutes and again washed 2× with clean PBS. The cells were blocked using 1% donkey serum in PBS overnight. After overnight blocking, the dishes were mixed with anti-Salmonella LPS antibody (abcam ab8274) in 0.5% bovine serum albumin (BSA) solution at a 1:200 dilution for 2 hours and then washed 3× with PBS. Mouse secondary antibodies bound to Alexa555 were added in 0.5% BSA solution at a 1:500 dilution for 2 hours and then washed 3× with PBS, with the last wash being an overnight wash. After overnight washing, the dishes were mixed with anti-Salmonella LPS antibody (Thermo Fisher Scientific, PA1-20811) in 0.5% bovine serum albumin (BSA)+0.125% Triton™ X-100 solution at a 1:200 dilution for 2 hours and then washed 3× with PBS. Rabbit secondary antibodies bound to Alexa488 were added in 0.5% BSA+0.125% Triton™ X-100 solution at a 1:500 dilution for 2 hours and then washed 3× with PBS, counterstained with DAPI and actin for 30 min in the UV and Cy5 channels, and washed 3× with PBS. Imaging of the various samples was performed using an Olympus IX83 fully motorized scope using an oil-dipping 100×objective. Only the SalTy T3-ADAS (MACH269) were capable of inducing actin ruffling in HDFn cells (FIGS. 1A and 1B).

### Example 4: Endogenous Effectors Delivered Via T3-ADAS Induce Cytotoxicity in Mammalian Cells

The 4 T3Es delivered by V. parahaemolyticus' T3SS profoundly disrupt the actin cytoskeleton and mediate rapid cytotoxicity upon translocation into eukaryotic cells. Vp-ADAS are purified from MACH625 cultured under T3SS-inducing conditions (DMEM) or MACH625 derivatives engineered to overexpress T3SSs via overexpression of the transcriptional activator exsA or deletion of the T3SS anti-activator exsD. In parallel, ADAS are purified from T3-deficient versions of these strains (e.g., strains in which a gene necessary for the expression, assembly, or function of the T3SS has been inactivated or repressed) and from MACH625 derivative lacking V. parahemolyticus's 4 T3Es. ADAS are resuspended in cell culture medium and co-incubated with eukaryotic cell cultures at different ratios and for different durations. Cytotoxicity is measured at various time points by measuring the number of live cells remaining (e.g., via trypan blue exclusion, LDH release assays, MTS assay, CellTiterGlo assay, etc.). ADAS purified from T3-expressing strains induce cell death to a greater extent than ADAS purified from T3-deficient strains of V. parahameolyticus' lacking type III effector proteins.

### Example 5: Engineering Heterologous T3-ADAS

T3SS genetic elements are well-defined for a diverse set of bacterial species that naturally encode T3SS, including a variety of enteric pathogens (Hu et al., Environmental Microbiology, 19(10): 3879-3895). The ˜15-35 kb of DNA encoding the structural genes required for these multi-protein nanomachines are typically found within large operons or as part of pathogenicity islands on the bacterial chromosome or virulence plasmid. This example describes three strategies for mobilizing T3SS genes, and their corresponding transcriptional activators, into a heterologous, non-T3 encoding host bacterium. E. coli is used as a model host bacterium. When combined with the ΔminCDE disruptions described in Example 1, these engineered bacterial strains produce heterologous T3-ADAS (i.e., ADAS comprising heterologous type 3 secretion systems).

A. Synthesis of T3-ADAS with Heterologous T355 on an Episomal Plasmid

At least two approaches can be pursued to generate episomally encoded heterologous T3SSs, depending on the source of the genetic material. Due to the large size of DNA to be mobilized, separate approaches are required depending on whether the T3SS operons are present on a virulence plasmid (e.g., as in Shigella flexnen), or on the chromosome (e.g., Vibrio parahaemolyticus). Shigella and Vibrio are provided as exemplary T3-encoding genes. The same approaches are applied to any host organism encoding a T3SS, e.g., any bacterium listed in Table 7.

TABLE 7
Bacterial genera having sequences encoding T3SS
Acetobacter
Achromobacter
Acidovorax
Aeromonas
Agrobacterium
Algicola
Anaeromyxobacter
Archangium
Arsenophonus
Azohydromonas
Bacteroides
Bilophila
Bordetella
Bradyrhizobium
Brenneria
Brevundimonas
Burkholderia
Caenimonas
Castellaniella
Caulobacter
Cedecea
Chelativorans
Chitinimonas
Chlamydia
Chiamydophila
Chloracidobacterium
Chromobacterium
Citrobacter
Collimonas
Corallococcus
Cupriavidus
Cystobacter
Desulfatiglans
Desulfocurvus
Desulfospira
Desulfovibrio
Dickeya
Edwardsiella
Endoziocomonas
Ensifer
Entero bacter
Erwinia
Escherichia
Glomeribacter
Hafnia
Hahella
Hamiltonella
Herbaspirillum
Hyalangium
Hyphomicrobium
Janthinobacterium
Kozakia
Lawsonia
Leclercia
Lonsdalea
Luteimonas
Lysobacter
Marinomonas
Mesorhizobium
Methylibium
Microvirga
Morganella
Myxococcus
Neochlamydia
Ottowia
Paludibacterium
Pandoraea
Pantoea
Parachlamydia
Pectobacterium
Phaseolibacter
Photobacterium
Photorhabdus
Phyllobacterium
Polyangium
Proteus
Protochlamydia
Providencia
Pseudoalteromonas
Pseudogulbenkiania
Pseudomonas
Pseudovibrio
Ralstonia
Ramlibacter
Regiella
Rhizobium
Rhodomicrobium
Rouxiella
Salmonella
Serratia
Shewanella
Shigella
Simkania
Sinorhizobium
Sodalis
Sphingomonas
Succinatimonas
Succinimonas
Succinivibrio
Tanticharoenia
Thalassomonas
Thiorhodococcus
Variovorax
Verrucomicrobium
Vibrio
Waddlia
Xanthomonas
Yersinia
Yokenella

1. Synthesis of T3-ADAS with Heterologous T355 on an Episomal Plasmid Sourced from the Shigella flexneri Virulence Plasmid

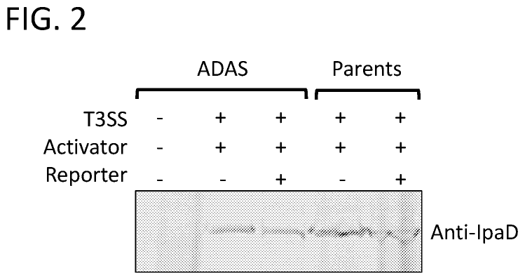

The plasmid capture methodology approach described in Reeves and Lesser, Bio-Protocol, 6(22): e2002, 2016 was used to generate the plasmid pT3SS (Table 3). pT3SS contains an episomal copy of the all the genes required for a functional Shigella flexneri T3SS, the absence of the majority of native T3 effectors (natural substrates of the T3SS), an RK2 origin of replication, and a kanamycin resistance cassette (Mou et al., PNAS, 115(25): 6452-6457, 2018). To express the heterologous type 3 secretion system from Shigella flexneri in an E. coli ADAS-producing strain, the plasmid pT3SS was transformed into the ΔminCDE E. coli strain, MACH060 (Table 2). To increase the abundance of T3SS on the surface of the E. coli ADAS, an IPTG-inducible plasmid (pVirB; Table 3) encoding virB (the Shigella flexneri T3SS transcriptional activator), an SC101 origin, and a spectinomycin resistance cassette is also introduced into the strain to generate MACH320 (Table 2). Presence of each introduced plasmid was confirmed by gene-specific PCR and growth on specific selective antibiotics. Expression of the T3SS in both parental and ADAS samples from MACH320 were demonstrated via an immunoblot probing for the T3-specific protein, IpaD (Creative Biolabs Inc. Catalog #EPAF-1108CQ): FIG. 2.

2. Synthesis of E. coli T3-ADAS with Heterologous T355 on an Episomal Plasmid Sourced from the Yersinia enterocolitica Virulence Plasmid

The plasmid capture methodology described above is also applied to transfer the T3SS genes from Yersinia enterocolitica into the ΔminCDE E. coli strain MACH060 (Table 2) on an episomal plasmid. The Y. enterocolitica transcriptional activator, LcrF (Table 5), is also introduced into the strain on an IPTG-inducible plasmid as a means to increase the abundance of the Y. enterocolitica T3SS on the surface. Expression of the T3SS in both parental and Ye-T3-ADAS samples are assessed via an SDS-PAGE gel analysis and Coomassie stain.

3. Synthesis of E. coli T3-ADAS with Heterologous T355 on an Episomal Plasmid Sourced from the Vibrio parahaemolyticus Chromosome

Restriction digest of genomic DNA and BAC technology are used as described in Akeda, et al., Biochem Biophys Res Commun, 427(2): 242-247, 2012 to ligate and package the T3SS operons from a multi-effector knockout strain of Vibrio parahaemolyticus (Vp) into a SuperCos 1 cosmid vector (Gigapack III Gold, Agilent). The resulting lambda phage particles are transduced into the E. coli ADAS-producing strain MACH060 (Table 2). Cosmids present in the resulting transductant colonies are sequenced at their 5′ and 3′ ends to confirm the presence of the Vp T3 operons in the insert sequence. To control expression of the heterologous Vp T3SS operons in the ADAS-producing strain, an anhydrotetracyline-inducible plasmid encoding EsxA, the Vp T3SS1 transcriptional activator (Table 5), is also introduced into the strain. Expression of the T3SS in both parental and ADAS samples is confirmed via an immunoblot using anti-sera raised against the Vp T3SS structural proteins.

B. Synthesis of ADAS with Heterologous Shigella T3SS Via P1 Phage Transduction

MACH607 (Table 2) is a strain of DH10B E. coli harboring the structural genes from the Shigella T3SS operons, devoid of effector genes, in the atp/gidB loci on the chromosome and an adjacent kanamycin resistance cassette. Standard P1 transduction protocols (Thomason et al., Current Protocols, 79(1): 1.17.1-1.17.8, 2007) were applied to transfer the T3 operons and adjacent kanamycin resistance cassette into BW25113 E coli to generate MACH823 (Table 2). Next, lambda red recombination, as described in Example 1, was used to replace the minCDE loci with a chloramphenicol resistance cassette to create MACH875 (Table 2), an ADAS-producing E. coli strain with the heterologous Shigella T3 genes stably integrated into the chromosome. All chromosomal modifications were confirmed by PCR. The IPTG-inducible plasmid encoding virB (pVirB), the Shigella flexneri T3SS transcriptional activator, an SC101 origin, and a spectinomycin resistance cassette were also introduced into the strain to control expression of the T3 operons, generating MACH976 (Table 2).

C. Engineering Heterologous T3 ADAS Through DNA Synthesis

Modern DNA synthesis technology is capable of generating the large (˜15-35 kb) pieces of DNA required for T3SS expression. There are a variety of circumstances in which DNA synthesis offers advantages over capture and mobilization approaches, including, but not limited to, synthesizing T3 genes from a genetically intractable organism (e.g., Chlamydia); eliminating undesired embedded genes (for example, toxic effectors) or refactoring genetic regulatory networks; or optimizing codon usage between donor and recipient species.

DNA sequences from the donor T3-positive organism are identified from available bioinformatic sequence databases. Next, the entire coding region is synthesized by a commercial vendor and cloned into an episomally replicating plasmid, e.g., using Gibson isothermal assembly, T2S restriction-based, Yeast transformation-associated recombination or other standard cloning methods, to generate a large plasmid capable of propagation in E. coli species (Gibson et al., Nat Methods, 7(11): 901-903, 2010; Potapov et al., ACS Synth Biol, 7(11): 2665-2674, 2018). In some instances, the synthesized DNA contains ˜500 bp of flanking DNA that, in some embodiments, is homologous to a region of E. coli or other desired target organism chromosome to maintain the option of site-specific integration.

Iterations of vector design include synthesis of native sequence; refactoring of regulatory elements or operon structure; tuning of gene expression (transcription or translation); codon optimization for the heterologous host; removal of unnecessary genes (e.g., non-T3 related genes), effectors (e.g., off-target genes), or deleterious genes (e.g., repressors); addition of elements such as transcriptional insulators, biochemical handles (e.g., reporters, affinity tags) or cloning elements (e.g., restriction sites, primer sites, barcodes, integrase sites); and swapping of genes with metagenomic homologs that alter performance of the T3SS (e.g., tip proteins).

### Example 6: Intracellular Protein Delivery to Mammalian Cells Via Heterologous T3-ADAS

The ability to deliver intracellular proteins to mammalian cells enables a variety of applications in human therapeutics. This example demonstrates that a heterologous protein can be delivered to human fibroblast cells using T3-ADAS. E. coli ADAS with a Shigella T3SS are used as exemplary ADAS, and a beta-lactamase reporter fusion protein is used as an exemplary cargo. This model could be applied to any number of animals, plant, or fungal applications in which delivery of an intracellular protein could have an effect on the target organism.

In this experiment, Normal Human Dermal Fibroblast, neonatal (HDFn) cells are loaded with a CCF4/AM, a fluorescence resonance energy transfer (FRET)-based dye that accumulates within the cytosol, such that the cells emit a green fluorescence at 518 nm that is detected on a BioTek® microplate reader. When an effector-B-lactamase fusion protein (OspB-TEM1) is delivered (translocated) into the cytosol of HDFn cells via type 3 secretion, the CCF4/AM substrate is cleaved, disrupting FRET and resulting in cells that emit blue fluorescence at 447 nm. The ratio of blue:green (447 nm/517 nm) signal is calculated and is indicative of the proportion of cells that received functional protein (OspB-TEM1).

1. Protein Secretion System Tag Fusion

To enable a functional readout of T3-specific translocation of a protein from T3-ADAS into HDFn cells, a plasmid encoding a previously verified IPTG-inducible effector beta-lactamase reporter fusion protein (pOspB-TEM1; Table 3), a ColE1 origin of replication, and a carbenicillin resistance cassette (Reeves et. al., ACS Syn Bio, 4: 644-654, 2015) was introduced into MACH320 to generate MACH435 (Table 2). As a control, the reporter plasmid was also introduced into MACH060 to generate MACH434 (Table 2), a wild-type ADAS-producing strain background (ΔminCDE) that does not contain the type 3 secretion expression system plasmids.

A. Preparation and Purification of E. coli ADAS with T3SS from Shigella

E. coli ADAS having T3SS from S. flexneri were prepared using methods described in Example 5, with a slight modification to the growth conditions. IPTG was added to the culture to a final concentration of 1 mM to induce expression of both the T3SS apparatus and the OspB-TEM1 beta-lactamase reporter protein. At the final ADAS purification step, T3SS-expressing ADAS are resuspended in mammalian cell culture media (Lonza's Dulbecco's Modified Eagle Medium (DMEM); Catalog No. 12-604F) supplemented with fetal bovine serum at 1% and 1 mM IPTG and quantified on the Spectradyne® nCS1™ Nanoparticle Analyzer, as described above.

B. Intracellular Protein Delivery Via T3-ADAS

Normal Human Dermal Fibroblasts, neonatal (HDFn) cell lines were cultured in accordance with ATCC instructions. In brief, cells were cultured at 37° C., 5% CO₂ in Corning® tissue culture flasks (T-75, T-150, or T-225, Catalog No. 430641) with Lonza's Dulbecco's Modified Eagle Medium (DMEM) (Catalog No. 12-604F) supplemented with fetal bovine serum at 10% and 1% penicillin-streptomycin. To harvest HDFn cells for the delivery assay, cells were washed with PBS without divalent cations (Ca²⁺ and Mg²⁺) and 2-3 mL of Gibco's TrypLE solution (Catalog No. 12605036) was added to cell surface for 5-10 minutes. Enzyme activity was quenched with 6-8 mL of complete growth media. After harvesting, the cells were counted using an automated cell counter (LUNA-II™ Automated Cell Counter, Logos Biosystems) and live/dead stain (Trypan Blue 0.4%). Black 96-well plates with a clear glass bottom were seeded at a density of 2×10⁴ cells/well approximately 20 hours prior to exposure with ADAS.

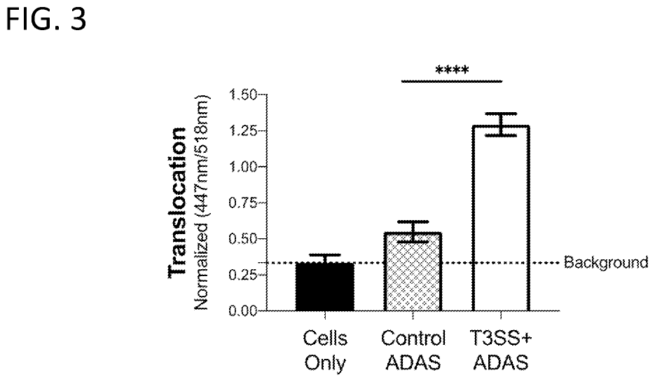

To confirm transfer of the OspB-TEM1 reporter protein from ADAS into the cytoplasm of HDFn cells, we performed a translocation assay. ADAS from MACH434 and MACH435 (Table 2) were prepared and purified as described in Example 9 and resuspended to a concentration of 2×10¹⁰ particles/mL in DMEM containing 1% FBS and 1 mM IPTG. The cell culture media was removed from the 96 well plate containing HFDn cells, cells were washed twice with PBS, and 100 μL of the ADAS sample (corresponding to 2×10⁹ particles) were added per well. Plates were centrifuged at 2000×g for 10 minutes to facilitate association of ADAS with the HFDn cells and then incubated at 37° C., 5% CO² for 4 hours. After co-incubation, ADAS were removed from the wells using 3 PBS washes, and cell culture media containing CCF4/AM dye from a LiveBLAzer™ FRET-B/G Loading Kit was added to the wells (Thermo Fisher Scientific catalog #K1095). After 20 minutes at 37° C. and 5% CO2, the CCF4/AM dye was removed with two PBS washes and replaced with 4% paraformaldehyde (PFA) in PBS to fix the samples. After 10 minutes, the PFA was removed and replaced with PBS. The plate was then analyzed for fluorescence on the BioTek® microplate reader (excitation: 409 nm, emission: 447 nm (blue) and 518 nm (green)). FIG. 3 shows the results of the transfer assay expressed as the ratio of blue to green signal intensity (447 nm/518 nm), which corresponds to the amount of CCF4/AM dye cleaved by TEM1 Beta-lactamase. A higher ratio of blue/green signal is interpreted as a larger amount of beta-lactamase protein delivery into HDFn cells. The results indicate that while MACH434 (no T3SS) shows a slight signal above background, MACH435 displays a highly statistically significant increase in blue/green signal ratio above the control ADAS (One-way ANOVA with Tukey's Multiple Comparisons Test, p<0.0001). This result shows that OspB-TEM1 protein was effectively transferred into HDFn human cells via the T3SS present in the ADAS. Notably, as beta-lactamase proteins are not native substrates of type 3 secretion systems, these results demonstrate the capacity of T3-ADAS to deliver heterologous cargo to human cells.

### Example 7: Intracellular Protein Delivery to Mammalian Cells Via Heterologous and Endogenous T3-ADAS

The ability to deliver proteins into mammalian cells enables a variety of applications in human therapeutics. This example demonstrates that a heterologous protein can be delivered to human cells from ADAS that harbor heterologous or endogenous T3SSs. Distinct laboratory (DH10b) and uropathogenic (CFT073) strains of E. coli ADAS with a Shigella T3SS exemplify ADAS bearing a heterologous T3SS, while enteropathogenic (LT2) S. enterica sp. Typhimurium ADAS exemplify ADAS bearing an endogenous T3SS. A beta-lactamase reporter fusion protein, OspB-TEM1, is used as an exemplary cargo. These models could be applied to any number of animals, plant, or fungal applications in which delivery of an intracellular protein could have an effect on the target organism.

In this experiment, HeLa cells are loaded with CCF2/AM, a forster resonance energy transfer (FRED-based dye that accumulates within the cytosol, such that the cells excited at 410 nm emit a green fluorescence at 520 nm that is detected on a SPECTRAMAX® microplate reader. When an effector-B-lactamase fusion protein (OspB-TEM1) is delivered (translocated) into the cytosol of HeLa cells via type 3 secretion, the CCF2/AM substrate is cleaved, disrupting FRET and resulting in cells that emit blue fluorescence at 450 nm. The ratio of blue:green (450 nm/520 nm) signal is calculated and is indicative of the magnitude of cytosolic delivery of functional protein (OspB-TEM1).

1. Protein Secretion System Tag Fusion

To enable a functional readout of T3-specific translocation of a protein from T3-ADAS into HeLa cells, a plasmid encoding a previously verified IPTG-inducible effector beta-lactamase reporter fusion protein (pOspB-TEM1), a ColE1 origin of replication, and a carbenicillin resistance cassette (Reeves et. al., ACS Syn Bio, 4: 644-654, 2015) was introduced into T3SS+ ADAS-producing strains. As a control, the reporter plasmid was also introduced into T3SS− ADAS-producing strain background that either does not contain heterologous type 3 secretion system expression plasmids or that does not contain an activator of endogenous T3SS expression.

A. Preparation and Purification of E. coli ADAS with T3SS from Shigella

T3SS+ and T3SS− ADAS from DH10b, LT2, and CFT073 were prepared using methods described in Example 5, in media supplemented with antibiotics and inducers described in table: “t3_ip_figures”. At the final ADAS purification step, T3SS+ and T3SS− ADAS are resuspended in PBS and quantified on the Spectradyne® nCS1™ Nanoparticle Analyzer, as described above.

B. Intracellular Protein Delivery Via T3-ADAS

HeLa cells were cultured in accordance with ATCC instructions. In brief, cells were cultured at 37° C., 5% CO₂ in Corning® tissue culture flasks (T-75, T-150, or T-225, Catalog No. 430641) with Lonza's Dulbecco's Modified Eagle Medium (DMEM) (Catalog No. 12-604F) supplemented with fetal bovine serum at 10% and 1% penicillin-streptomycin. To harvest HeLa cells for the translocation assay, cells were washed with PBS without divalent cations (Ca²⁺ and Mg²⁺) and 2-3 mL of Gibco's TrypLE solution (Catalog No. 12605036) was added to cell surface for 5-10 minutes. Enzyme activity was quenched with 6-8 mL of complete growth media. After harvesting, the cells were counted using an automated cell counter (LUNA-II™ Automated Cell Counter, Logos Biosystems) and live/dead stain (Trypan Blue 0.4%). Black 96-well plates with a clear glass bottom were seeded at a density of 2×10⁴ cells/well approximately 20 hours prior to exposure with ADAS.

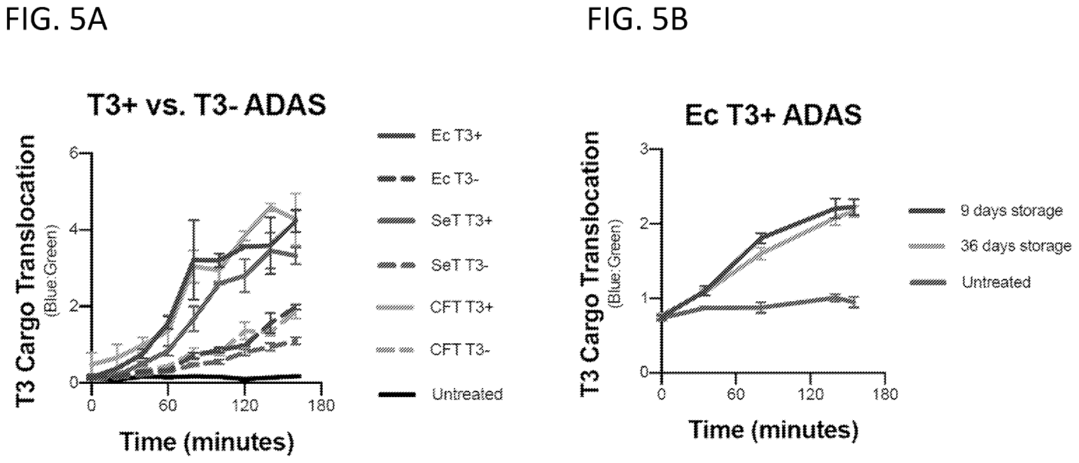

To confirm transfer of the OspB-TEM1 reporter protein from ADAS into the cytoplasm of HeLa cells, we performed a translocation assay. First, the cell culture media was removed from the 96 well plate containing HeLa cells, cells were washed twice with PBS, and cell culture media containing CCF2/AM dye from a LiveBLAzer™ FRET-B/G Loading Kit was added to the wells (Thermo Fisher Scientific catalog #K1095). The plate was wrapped in foil, to protect the dye from light, and incubated at room temperature for 60 minutes. During the incubation, ADAS were resuspended to a concentration of 4×10⁹ particles/mL in PBS. The CCF2/AM dye was removed, and the cells were washed three times with PBS. 50 μL of the ADAS sample (corresponding to 2×10⁸ ADAS) were added per well, and the plate was then analyzed for fluorescence on a SPECTRAMAX® microplate reader (excitation: 410 nm, emission: 450 nm (blue) and 520 nm (green)). FIGS. 5A and 5B show the results of the transfer assay expressed as the ratio of blue to green signal intensity (450/520 nm), which corresponds to the amount of CC24/AM dye cleaved by TEM1 Beta-lactamase, over time. A higher ratio of blue/green signal is interpreted as a larger amount of beta-lactamase protein delivery into HeLA cells. The results indicate that T3SS− ADAS show a slight signal above background that increases over time; however, T3SS+ ADAS display a larger increase in blue/green signal ratio above the T3SS− ADAS. This result shows that OspB-TEM1 protein was effectively transferred into HeLa cells via T3SSs present in the ADAS. Notably, as beta-lactamase proteins are not native substrates of type 3 secretion systems, these results demonstrate the capacity of T3-ADAS to deliver heterologous cargo to human cells.

C. T3-ADAS Remain Capable of Intracellular Protein Delivery after Prolonged Storage at 4° C.

To assess the durability of T3-ADAS, intracellular protein delivery of recently purified T3SS+ ADAS was assessed relative to T3SS+ ADAS subjected to prolonged storage at 4° C. (9 days vs. 36 days). The translocation assay was performed as described above; however, a slightly higher number of T3SS+ ADAS was added to each well (5×10⁸ vs. 2×10⁸). The results indicated that T3SS+ ADAS retain the capacity to deliver heterologous cargo to human cells even after prolonged storage at 4° C. (FIG. 5B).

### Example 8: T3-ADAS with Enhanced Expression of Effectors

The known repertoire of T3SS natural effector substrates is well defined, and continues to expand. Known and predicted bacterial effectors are provided, for example, at the SecretEPDB (Bacterial Secreted Effector Protein Database) provided by Monash University. These effectors are targeted to the T3SS machinery and transverse the needle to be delivered into the cytoplasm of a host cell or into the extracellular milieu. The conserved nature of T3SS structural proteins means that effectors present in one T3-encoding strain can often be promiscuously secreted through the T3SS of another species. For example, the Salmonella effectors SipA, SopA, SopE1, and SopE2 are all successfully targeted to and secreted by the T3SS in Shigella flexneri (Costa et al., mBio, 3(1): e00243-11, 2012). In this example, we demonstrate the enhanced expression and loading of both native and non-native T3 effectors into heterologous T3-ADAS and endogenous T3-ADAS and detail strategies for evaluating and confirming effector secretion.

This approach can be applied to any protein known or predicted to be a T3-secreted substrate.

A. Expression and Loading of Native and Non-Native Effectors in T3-ADAS with Heterologous T3SS

To enable a functional readout of effector expression and type 3 specific secretion in T3-ADAS, the effectors OspF (Shigella flexneri), and NIeC (EPEC) were cloned into an expression vector. The sequence of each effector was codon-optimized for expression in E. coli, fused to an C-terminal FLAG-tag, and cloned into a plasmid containing the TetA promoter, the TetR repressor (which represses the TetA promoter), a CloDF origin of replication, and a carbenicillin resistance gene for antibiotic selection to generate PRO295 and PRO292, respectively (Table 3). Each plasmid was introduced into MACH320 (ΔminCDE, encoding the Shigella flexneri T3SS and transcriptional activator VirB on plasmids) to generate MACH707 and MACH704 (Table 2). As a T3SS null control, the NIeC-expressing construct was also introduced into MACH060 (ΔminCDE) to generate MACH778 (Table 2).

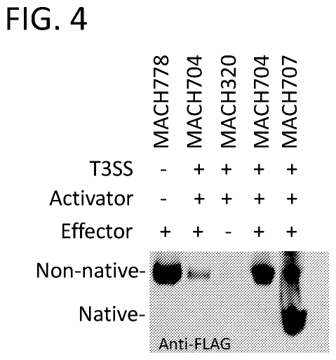

Each of these strains were grown overnight in 1 L of Luria Broth supplemented with anhydrotetracycline (1000 ng/mL) inducer. ADAS were then derived from these strains and purified as indicated in Example 10. The purified ADAS were subjected to the nanoparticle characterization and viable cell plating procedures described in Example 12. CFU plating revealed that the concentration of viable cells present within the purified population of ADAS was at or below the limit of detection (100 CFU/mL). ADAS were centrifuged at 20,000×g for 10 minutes at 4° C. The supernatant was removed and the pelleted ADAS were resuspended in Lysis buffer (1× BugBuster™ Protein Extraction Reagent (MilliporeSigma™) with 1×LDS from ThermoFisher) and heated at 85° C. for 2 minutes. An equal volume of lysed ADAS was then loaded into a 4-12% BisTris polyacrylamide gel. Proteins on the gel were resolved, transferred to a nitrocellulose membrane, and incubated in Intercept PBS blocking buffer for 60 minutes at room temperature. The membrane was incubated with primary antibody (Rabbit anti-FLAG at a 1:5000 dilution) for 60 minutes at room temperature. Antibodies were resuspended in Intercept PBS blocking buffer supplemented with 0.2% Tween20. The membrane was subsequently washed three times in 1×PBS+0.05% TWEEN® 20 followed by incubation in INTERCEPT® PBS blocking buffer (LI_COR®)+0.2% TWEEN® 20 supplemented with secondary antibodies (Goat anti-Rabbit 680 both at a 1:5,000 dilution). The membrane was washed three times in 1×PBS+0.05% Tween20 and imaged on an Invitrogen™ iBright™ Imager (Thermo Fisher). FIG. 4 demonstrates that both ADAS and T3-ADAS were loaded with the respective T3 effector-FLAG tagged protein, confirming that cargo proteins from at least two T3-encoding species are stably loaded into heterologous T3-ADAS.

B. Secretion of Native and Non-Native Effectors from Heterologous T3-ADAS

Heterologous T3-ADAS that were pre-loaded with either native or non-native effector-FLAG fusion proteins were analyzed on a modified version of a solid-plate-based secretion assay (Ernst et al., mBio, 9(3): e01050-18, 2018). A nitrocellulose (NC) membrane was overlaid onto a solid Congo red (CR)-containing media containing 1 mM IPTG and 1000 ng/mL anhydrotetracycline to continue induction of the T3SS and cargo on plates. Purified T3-ADAS samples were concentrated to −10¹% DAS/mL, and 10 μL samples were spotted onto the plates and allowed to dry completely, then incubated at 37° C. overnight. Membranes were removed, washed, and probed with a Rabbit-anti-FLAG primary antibody, cytoplasmic (lysis) control Mouse-anti-DnaK antibodies, and corresponding secondary antibodies, and developed on an Invitrogen™ iBright™ Imager (Thermo Fisher) as described above. Proteins detected on the imager are the result of T3-specific secretion of the effectors from the T3-ADAS and stable binding onto the nitrocellulose membrane. The lack of signal from the DnaK control antibody confirms the effector signal is not due to lysis of the ADAS and confirms that both native and non-native effectors can be secreted from the heterologous T3-ADAS.

C. Expression and Loading of Native and Non-Native Effectors in T3-ADAS

A similar approach to loading heterologous T3-ADAS is applied to Salmonella-derived T3-ADAS. The codon-optimized sequences of native (SopE) and non-native (OspF from Shigella flexneri) effectors are fused to a C-terminal FLAG-tag and cloned into an anhydrotetracycline inducible expression plasmid containing a CloDF origin of replication and a carbenicillin resistance gene for antibiotic selection (Table 3). Each plasmid is introduced into MACH269, a T3-ADAS producing Salmonella enterocolitica ΔminCDE strain containing a plasmid encoding the Salmonella T3SS transcriptional activator HilA on a plasmid (Table 2). As a T3SS null control, the same constructs are introduced into a Salmonella enterocolitica ΔminCDE ΔinvA double mutant, which can produce ADAS, but does not express the T3SS. ADAS pellets are generated from strains grown in the presence of inducer overnight and analyzed by immunoblotting for the FLAG tagged effector cargo.

D. Secretion of Native and Non-Native Effectors from T3-ADAS

Secretion from SalTY T3-ADAS is confirmed using a method similar to that described in Example 8B. Purified SalTy T3-ADAS and invA(T3)-knockout ADAS samples and are concentrated to ˜10¹⁰ ADAS/mL, and 10 μL samples are spotted onto membranes overlaid on top of high-salt LB plates and allowed to dry completely. Following overnight incubation at 37° C., membranes are probed for the presence of T3-secreted effectors.

### Example 9: Translocating a T3-Independent Cargo Via T3-ADAS

T3SS substrates (e.g., native effectors, non-native effectors, or heterologous cargoes) are targeted to the T3SS machinery via an N-terminal secretion signal. Such secretion signals can be highly variable in amino acid sequence and length (varying from 15-180 amino acids), although typically composed of a relatively unstructured or disordered region (Arnold et al., PLoS Pathog., 5(4): e1000376, 2009) and in some cases a chaperone binding domain. Exemplary secretion signals are provided in Table 8. Exogenous cargo proteins (non-native T3 substrates, e.g., exogenous cargoes, e.g., exogenous proteins or peptides) can be modified to become substrates of a T3SS by fusing a type 3 secretion signal to their N-terminus. When moieties comprising a signal sequence (e.g., native effectors, non-native effectors, or heterologous cargoes modified to comprise a signal sequence) are expressed in T3SS-encoding strains, the moieties can be secreted extracellularly and delivered to the cytoplasm of target cells (e.g., eukaryotic cells) (Walker et al., Expert Rev Mol Med, 19: e6, 2017). This example describes approaches for fusing T3 secretion signals from known T3 effectors onto T3-independent proteins and demonstrating translocation into eukaryotic cells.

TABLE 8
Secretion signals

| Organism | Secretion signal | Amino acid length | Sequence |
|---|---|---|---|
| Salmonella | SptP | 180 | SEQ ID NO: 29 |
| Salmonella | SopD | 50 | SEQ ID NO: 30 |
| Salmonella | SipA | 180 | SEQ ID NO: 31 |
| Salmonella | slrP | 200 | SEQ ID NO: 32 |
| Salmonella | SopA | 100 | SEQ ID NO: 33 |
| Salmonella | SopB | 180 | SEQ ID NO: 34 |
| Salmonella | SipC | 180 | SEQ ID NO: 35 |
| Salmonella | AvrA | 100 | SEQ ID NO: 36 |
| Salmonella | OspB | 288 | SEQ ID NO: 37 |
| Salmonella | SopE2 | 120 | SEQ ID NO: 38 |
| Shigella | IpaH9.8 | 60 | SEQ ID NO: 39 |
| Shigella | OspC3 | 60 | SEQ ID NO: 40 |
| Shigella | OspG | 60 | SEQ ID NO: 41 |
| Shigella | IpaH4.5 | 60 | SEQ ID NO: 42 |
| Shigella | IpaH7.8 | 60 | SEQ ID NO: 43 |
| Pseudomonas | ExoS | 54 | SEQ ID NO: 44 |
| Yersinia | yopE | 50 | SEQ ID NO: 45 |

A. T3 Secretion Signal Fusions to the T3-Independent Protein, Beta-Lactamase (TEM)

To demonstrate translocation of a T3-independent protein into a eukaryotic cell, a series of secretion signal-Beta-lactamase (TEM) fusions from representative T3-encoding species are designed and synthesized. One such fusion is OspC3 (50aa)-TEM, in which the first 150 bp (50 amino acids) of the OspC3 coding sequence, which contains the N-terminal T3 secretion signal, is fused to the entire open reading frame of TEM. The construct also includes a ribosome binding site and flanking sequences that provide Type 2S cloning compatibility with PRO055 (Table 3), an expression construct containing the TetA promoter, the TetR repressor (which represses the TetA promoter), a pMB1 origin of replication and a kanamycin resistance gene for antibiotic selection, and a Bsal T2S cloning site. Cloning OspC3(50aa)-TEM into PRO055 yields the plasmid pOspC3(50aa)-TEM (Table 3).

Versions of the above-described TEM fusion are made for several representative T3 encoding species: SopE (104aa)-TEM from Salmonella, ExoS (54aa)-TEM from Pseudomonas, and YopE(50aa)-TEM from Yersinia, to generate the following plasmids, respectively; pSopE(104aa)-TEM, pExoS(54aa)-TEM, pYopE(50aa)-TEM (Table 3). Constructs are verified by sequencing analysis.

Each construct is introduced into MACH320, the heterologous T3-ADAS producing strain, via electroporation. Additionally, each construct is introduced into its T3-ADAS producing cognate host; for example, pSopE(104aa)-TEM is introduced into MACH269, the SalTy T3-ADAS producing strain described above (Table 2).

B. Translocation of T3-independent proteins (secretion-signal TEM fusions) by heterologous T3-ADAS and endogenous T3-ADAS To demonstrate intracellular delivery (translocation) of the TEM fusion proteins, heterologous T3-ADAS expressing the TEM fusion proteins described in Example 9A were prepared using the methods outlined in Example 10 and evaluated using the CCF4-BLAZer assay as described in Example 7.

A similar approach is used for cognate secretion signal-TEM fusion and host T3SS pairings: for example, Shigella T3-ADAS are loaded with OspC3(50aa)-TEM, SalTy T3-ADAS are loaded with SopE(104aa)-TEM, Yersinia T3-ADAS are loaded with YopE-TEM, and Pseudomonas ADAS are loaded with ExoS(54aa)-TEM.

C. T3 Secretion Signal Fusions to T3-Independent Effectors from Type IV or Type VI Secretion Systems.

Bacterial Type IV and Type VI secretion systems are functionally and genetically distinct from T3SSs, each having their own repertoire of effectors. To demonstrate that effector substrates from Type IV and Type VI secretion systems can be retargeted and translocated by the T3SS upon fusion to a T3 secretion signal, representative effectors from the Type IV secretion system of Legionella pneumophila SdhA (Laguna et al., PNAS, 103(49): 18745-18750, 2006) and the type VI secretion system of Edwardsiella tardis (EvpP) (Chen et al., Cell Host and Microbe, 21: 47-58, 2017) are fused to the N-terminal OspC3 (50aa) secretion signals and a C-terminal FLAG tag, then cloned into an expression construct as described in Example 9A and sequence verified.

Each construct is introduced into MACH320, the heterologous T3-ADAS producing strain, via electroporation.

D. Translocation of T3 Secretion Signal Fusions to T3-Independent Effectors from Type IV or Type VI Secretion Systems by Heterologous T3-ADAS

Translocation of the FLAG-tagged effectors was evaluated using the methods detailed in Carleton et al., Nature Communications, 4: Article number 1590, 2013. Heterologous T3-ADAS preloaded with secretion signal-effector-FLAG fusion proteins were purified as described in Example 1 and co-incubated with HDFn cells for 2.5 hours. HDFn cells were washed thoroughly and lysed with 0.1% Triton™ X-100. Lysates are filtered with 0.2 micron filters to remove any remaining T3-ADAS and evaluated with an anti-FLAG immunoblot.

### Example 10: Translocating a T3-Independent Cargo Via T3-ADAS

This example describes methods of purifying populations of ADAS from a culture of an ADAS-producing strain. ADAS were purified from high cell density cultures of ADAS-producing strains via combinations of 1) low speed centrifugation, 2) selective outgrowth, and 3) buffer exchange/concentration. Low speed centrifugation procedures were used to selectively deplete viable cells and large cellular debris, while enriching ADAS in a mixed suspension. Selective outgrowth procedures were used to reduce the number of viable cells present in the sample via the addition of compounds that are directly anti-microbial and/or compounds that enhance viable cell sedimentation via low speed centrifugation. Buffer exchange/concentration procedures were used to transition ADAS from larger volumes of bacterial culture media into smaller volumes of 1×PBS while removing culture additives and cellular debris.

A. ADAS Purification

ADAS-producing strains were generated using the molecular cloning procedures described in Examples 1 and 2, then cultured to high cell density in culture medium (1 to 1000 mL).

Cultures were transferred to centrifuge tubes and subjected to a low speed centrifugation procedure aimed at pelleting intact cells and large cell debris while maintaining ADAS in the supernatant. Low speed centrifugation procedures were performed either at 4° C. or at room temperature. In some instances, the low speed centrifugation procedure was a sequence of sequential 10-minute spins at 1,000×g, 2,000×g, 3,000×g, and 4,000×g performed on an Allegra X14R benchtop centrifuge or an Eppendorf 5424 R benchtop centrifuge. In some instances, the low speed centrifugation procedure consisted of sequential 2,000×g spins for 20 minutes at 4° C. in which the supernatant of the first spin was decanted into a sterile centrifuge bottle prior to the second spin. In some instances, the low speed centrifugation procedure was a single 40-minute spin at 4,000×g in a Sorvall Lynx 6000 floor centrifuge in which the rate of rotor acceleration was set to the lowest possible setting.

Following low speed centrifugation, culture supernatants were decanted into sterile culture tubes and subjected to a selective outgrowth. In some instances, concentrated antibiotic solutions (e.g., ceftriaxone, kanamycin, carbenicillin, gentamicin, ciprofloxacin) or other concentrated chemical solutions (e.g., sodium chloride, sodium hydroxide, M hydrochloric acid, glucose, cas-amino acids, and D-amino acids) were added directly to the culture supernatants. In other cases, the culture supernatants were pelleted via high speed centrifugation for 5 to 60 minutes at 10,000×g to 20,000×g and the pellets were resuspended in fresh culture media containing inhibitory concentrations of antibiotics or other chemical solutions. Selective outgrowth was performed by incubating ADAS at 4° C. to 42° C. for 1 to 3 hours with agitation at 250 rpm. ADAS were then transferred to sterile centrifuge tubes and subjected to an additional round of low speed centrifugation for 15 minutes at 4° C., 4,000×g.

Following selective outgrowth and low speed centrifugation, supernatants were subjected to buffer exchange/concentration procedures. In some instances, this was carried out by passing supernatants over a 0.2 μm aPES filter followed by 1 to 9 volumes of 1×PBS. In other cases, ADAS were pelleted via centrifugation at 10,000×g to 20,000×g for 5 to 60 minutes, washed in 1 to 9 volumes of 1×PBS, pelleted again, and resuspended in 1×PBS at 1 to 100,000×concentration from the starting culture volume.

### Example 11: Methods for Negative Selection of Viable Parent Contaminants

A. ADAS Purification Using Antibiotic Selection

In some instances, concentrated antibiotic solutions (e.g., ceftriaxone, kanamycin, carbenicillin, gentamicin, and/or ciprofloxacin) are added to an ADAS-containing preparation to deplete viable parent bacteria. Ceftriaxone is a beta-lactam class of antibiotic that is resistant to beta-lactamases typically used for plasmid selection and maintenance. Beta-lactams target expanding cell walls—a hallmark of actively growing cells. This selection can be applied to mixtures of ADAS and viable parents to selectively deplete the actively-growing viable parents. Briefly, concentrated ceftriaxone (500 μg/mL final concentration) is added into the fresh media and the culture is allowed to grow at 37° C. shaking at 250 RPM for at least one hour, in some instances longer than one hour. Growing parent cells will either lyse or filament.

The growth-selected culture is centrifuged at a very low speed of 500×g for 10 minutes to deplete filamentous parent bacteria. The supernatant from this centrifugation cycle is aspirated and subjected to another round of centrifugation at 8,000×g for 20 minutes to separate the ADAS from lysed parent cell debris. The pellet is then buffer exchanged into appropriate buffer conditions. In some instances, this buffer is phosphate-buffered saline.

B. ADAS Purification Using Auxotrophy

ADAS-producing parental strains that are auxotrophic, i.e., are unable to synthesize an organic compound required for growth, are useful for the manufacturing of ADAS. Such strains are able to grow only when the organic compound is provided. Auxotrophic parental strains may thus be selected against by storing or incubating an ADAS preparation in media lacking the organic compound, thus providing an additional method for reducing parental burden in the ADAS preparation.

Auxotrophic ADAS-producing parental strains are constructed via standard Lambda-RED genome editing (Datsenko and Wanner, PNAS, 97(12): 6640-6645, 2000) in strains of Table 2. Briefly, primers were designed that amplified either a chloramphenicol or kanamycin cassette from plasmids pKD3 and pKD4, respectively. These primers encode ˜40 base pairs of homology to the target loci of the E. coli genome. In some instances, the target loci are dapA or thyA.

These linear fragments are purified and transformed into E. coli and plated on LB media containing appropriate antibiotic and auxotrophic nutrient −100 μg/mL diaminopimelic acid in the case of AdapA or 25 μg/mL thymidine in the case of AthyA. Resultant colonies are confirmed for their auxotrophic genotypes and phenotypes. From this point on, the auxotrophic nutrient must be maintained in the media to support growth of the auxotrophic parent. The antibiotic resistance is cured from the genotype using pCP20 as described in Datsenko and Wanner, PNAS, 97(12): 6640-6645, 2000. These cured auxotrophic strains are transformed with the appropriate T3SS-encoding plasmids to create the auxotrophic T3-ADAS-producing parents.

To prepare ADAS from auxotrophic parents, strains are grown according to Example 1B with the modification that the auxotrophic nutrient is included in the growth media as stated above. ADAS and contaminating parents are collected as above, washed once with PBS and recollected, and exchanged into a media depleted of the auxotrophic nutrient. These are then allowed to grow at 37° C. shaking at 250 RPM for at least two hours and, in some instances, longer than two hours. Growth in thymidine-less medium in the case of AthyA induces thymidine-less cell death. Growth in diaminopimelate-less medium in the case of AdapA results in cell wall rupture and lysis.

The growth-selected culture is centrifuged at a very low speed of 500×g for 10 minutes to deplete filamentous parent bacteria. The supernatant from this centrifugation cycle is aspirated and subjected to another round of centrifugation at 8,000×g for 20 minutes to separate the ADAS from lysed parent cell debris. The pellet is then buffer exchanged into appropriate buffer conditions. In some instances, this buffer is phosphate-buffered saline.

C. ADAS Purification Using Filamentation

In some instances, growth media can be adjusted so that cell size is modified via induction of filamentation in genome-laden viable parents. Highly halogenic medium (e.g., medium to which concentrated sodium chloride has been added) can induce the formation of bacterial filaments by disrupting the septum formation of bacteria (Mattick, K. L., et al (2000). Appl. Environ. Microbiol. 66, 1274-1279). Growth of viable parents in high salt solutions thus results in size differentiation of parent bacteria from ADAS. Briefly, to the fresh media containing the ADAS and viable parents a concentration sodium chloride solution is added to a final concentration of 5% w/v. The preparation is then incubated at 37° C. shaking at 250 RPM for at least two hours and, in some instances, longer than two hours.

The growth-selected culture is centrifuged at a very low speed of 500×g for 20 minutes to deplete filamentous parent bacteria, using a slow ramp speed to prevent co-precipitation of the smaller ADAS particles. The supernatant from this centrifugation cycle is aspirated and subjected to another round of centrifugation at 8,000×g for 20 minutes to separate the ADAS from lysed parent cell debris. The pellet is then buffer exchanged into appropriate buffer conditions. In some instances, this buffer is phosphate-buffered saline.

### Example 12: Characterization of T3-ADAS

This example describes methods of characterizing purified populations of ADAS and/or unpurified ADAS-producing bacterial cultures using a variety of approaches, including microscopy and immunofluorescence, nanoparticle characterization, viable cell plating, immunoblotting, and flow cytometry.

A. Viable Cell Plating

To determine the concentration of viable parent bacterial cells present before and after purification, the ADAS-producing culture and the purified ADAS population described herein were assayed via viable cell plating. Serial dilutions were prepared by repeatedly transferring 100 μL of either the ADAS-producing culture or the purified population of ADAS into 900 μL of 1×PBS. Then, 10 μL of each dilution was spotted on selective media and incubated at 37° C. to allow the growth of viable cells. After 24 hours, dilutions containing 1-100 colonies were counted, and this colony count was multiplied by the appropriate dilution factor to enumerate the number of colony forming units (CFU) per mL of sample (i.e., the concentration of viable cells present in each sample). Thus, the concentration of viable cells present in a sample can be enumerated via viable cell plating up to >100 CFU per mL.

B. Nanoparticle Characterization

A purified population of ADAS from Example 10 were suspended in 1×PBS, diluted to a concentration between 10⁷ and 10⁹ particles per mL, and TWEEN® 20 (Sigma Aldrich) was added to a final concentration of 0.1% (v/v) to minimize particle aggregation. This suspension of ADAS was diluted 20-fold and loaded onto a CS2000 cartridge (Technology® Supplies Ltd.), and analysis was performed on a Spectradyne® nCS1™ Nanoparticle Analyzer. The nCS1™ measures both the size and concentration of individual particles in a suspension by applying a bias voltage across a constriction of defined size and monitoring changes in electrical resistance as a function of time (Fraikin et al., Nature Nanotechnology, 6(5): 308-313, 2011). Roughly 50,000 particles within the range of 200 to 2,000 nm were measured. In other examples, the number of particles is between 10-100,000 particles. The resulting data were plotted as a cumulative size distribution, which reveals the concentration of particles between 200 and 2,000 nm in diameter in the suspension of purified T3-ADAS. Additionally, the data were exported as an integration range report, which enumerates the aggregate volume of 200 to 2,000 nm particles in the suspension of purified ADAS. Collectively, these data demonstrate that the concentration and size distribution of purified ADAS can be quantified via nanoparticle characterization methods. Additionally, quantification of small molecules (e.g., ATP, nucleic acids, or proteins (e.g., GFP) observed within a purified population of ADAS can be divided by the aggregate volume of ADAS or the number of particles present in the assay, enabling calculation of the average concentration of small molecules, nucleic acids, or proteins of interest within a purified population of T3-ADAS.

C. Light Microscopy and Immunofluorescence

A preparation of T3 ADAS was prepared as described in Examples 1 and 2. Separately, glass coverslip bottom 35 mm dishes from ibidi® were coated with a 0.01% poly-lysine solution for 5 minutes; the solution was aspirated and the dishes were allowed to dry. A dense suspension of purified or unpurified ADAS containing culture was placed on the coverslip bottom in 1 μL drops and allowed to almost dry. Then 1 mL of 2.8% paraformaldehyde/0.04% glutaraldehyde fixative was added for 30 minutes. Samples were washed 3× in PBS, permeabilized with 0.1% Triton™ X-100 (Sigma-Aldrich), washed 2× with PBS, further permeabilized with 10 μg/mL lysozyme, washed 2× with PBS, blocked with 1% donkey serum, washed 2× with PBS, stained with primary antibody (anti-E. coli LPS) or antibodies raised against specific T3 proteins (ex: Anti-IpaD (Creative Biolabs Inc. Catalog #EPAF-1108CQ), washed, and stained with a secondary antibody bound to Alexa Fluor® 555 (Thermo Fisher). The dishes were counter-stained with DAPI for 10 min and washed 3× with PBS. The final dishes were imaged using a 100×oil-dipping objective lens on an Olympus IX83 inverted microscope (Olympus). Images were rendered using the publicly available software package Fiji. Microscopy showed that that within the parent population, there are ADAS which have the LPS membrane stain, but lack a genome (no-or-low DAPI positive stain).

D. Immunoblotting Proteins in ADAS

To evaluate ADAS protein composition, the presence in ADAS of two housekeeping proteins, DnaK and GroEL, that are known to be present in parent E. coli bacterial cells was characterized. ADAS-producing strains are generated and subjected to the ADAS purification procedures described herein. In parallel, 5 mL aliquots of the ADAS-producing cultures were pelleted at 8,000×g and resuspended in a 5 mL of 1×PBS, then diluted 100-fold in 1×PBS. 4× NuPAGE™ LDS Sample Buffer (Thermo Fisher) was added to 100 μL aliquots of the diluted cultures and to the purified ADAS in order to lyse the samples. The various lysates were incubated at 85° C. for 2 minutes, then 40 μL of each sample was resolved on an SDS-polyacrylamide gel. A positive control, comprising 10 ng of purified recombinant E. coli GroEL protein (Abcam, ab51307) or recombinant DnaK protein (Abcam, ab51121), was also denatured in 1×LDS Sample Buffer at 85° C. for 2 minutes and resolved on the gel. Proteins were transferred to a nitrocellulose membrane that was blocked for 1 hour at room temperature in Intercept® (PBS) Blocking Buffer (LI-COR®) and incubated with a 1:1,000 dilution of an antibody targeting GroEL (Abcam, ab90522) or DnaK (Abcam, ab69617) at room temperature overnight. The blots were then washed in excess volumes of PBS+0.05% TWEEN® 20, incubated with anti-mouse (LI-COR®, 926-68070) and anti-rabbit (LI-COR®, 926-32211) antibodies conjugated to fluorescent dyes for 1 hour at room temperature, washed again in excess volumes of PBS+0.05% TWEEN® 20, and imaged on an Invitrogen™ iBright™ Imager (Thermo Fisher). Specific bands corresponding to GroEL and DnaK were present in the lanes containing lysates from ADAS-producing cultures and in the lanes containing lysates from purified ADAS; thus, the protein content of ADAS can be detected via immunoblotting.

To evaluate T3-ADAS protein composition, the above protocol is used with anti-FLAG antibodies (Abcam, ab21536) or antibodies raised against specific T3 proteins (e.g., Anti-IpaD (Creative Biolabs Inc. Catalog #EPAF-1108CQ).

E. SDS-PAGE

When T3-specific antibodies are not available, ADAS pellets can be evaluated on a standard SDS-PAGE gel. Comparison of T3-expressing samples vs T3-null samples are used to demonstrate the presence of T3 proteins under inducing conditions.

What is claimed is:

1. An achromosomal dynamic active system (ADAS) derived from a parent bacterial cell, the ADAS comprising a bacterial Type 3 secretion system (T3SS) that is heterologous to the parent bacterial cell.

2. The ADAS of claim 1, wherein the parent bacterial cell is a Gram-negative bacterial cell.

3. The ADAS of claim 1 or 2, wherein the parent bacterial cell does not comprise an endogenous T3SS.

4. The ADAS of claim 3, wherein the parent bacterial cell is an E. coli cell.

5. The ADAS of claim 4, wherein the E. coli cell is a Nissle E. coli cell.

6. The ADAS of any one of claims 1-5, wherein the parent bacterial cell is a probiotic cell.

7. The ADAS of any one of claims 1-6, wherein the T3SS is from a genus of Table 6.

8. The ADAS of claim 7, wherein the T3SS is a Salmonella T3SS, a Vibrio T3SS, an Escherichia T3SS, a Yersinia T3SS, a Shigella T3SS, a Pseudomonas T3SS, or a Chlamydia T3SS.

9. The ADAS of claim 8, wherein the Salmonella T3SS is a Salmonella enterica T3SS.

10. The ADAS of claim 8, wherein the Vibrio T3SS is a Vibrio parahaemolyticus T3SS.

11. The ADAS of claim 8, wherein the Escherichia T3SS is an enteropathogenic E. coli (EPEC) T3SS.

12. The ADAS of claim 8, wherein the Yersinia T3SS, is a Yersinia enterocolitica T3SS.

13. The ADAS of claim 8, wherein the Shigella T3SS is a Shigella flexneri T3SS.

14. The ADAS of any one of claims 1-13, wherein the parent bacterial cell comprises one or more heterologous nucleotide sequences encoding the components of the T3SS.

15. The ADAS of claim 14, wherein the one or more nucleotide sequences encoding the components of the T3SS are carried on a vector.

16. The ADAS of claim 15, wherein the parent bacterial cell has been transiently transformed with the vector.

17. The ADAS of claim 15, wherein the parent bacterial cell has been stably transformed with the vector.

18. The ADAS of any one of claims 1-17, wherein the parent bacterial cell further comprises a moiety that increases the level of the T3SS in the ADAS.

19. The ADAS of claim 18, wherein the moiety is a transcriptional activator of the one or more heterologous nucleotide sequences encoding a component of the T3SS.

20. An achromosomal dynamic active system (ADAS) derived from a parent bacterial cell, the ADAS comprising a bacterial Type 3 secretion system (T3SS) that is endogenous to the parent bacterial cell, wherein the parent bacterial cell has been modified to reduce the level of an endogenous protein or polypeptide capable of being secreted by the T3SS.

21. The ADAS of claim 20, wherein the parent cell bacterial has been modified by deleting a transcriptional activator of the endogenous protein or polypeptide capable of being secreted by the T3SS.

22. The ADAS of claim 20 or 21, wherein the parent bacterial cell is a Gram-negative bacterial cell.

23. The ADAS of any one of claims 20-22, wherein the parent bacterial cell is from a genus of Table 6.

24. The ADAS of claim 23, wherein the parent bacterial cell is a Salmonella species, a Vibrio species, an Escherichia species, a Yersinia species, or a Shigella species.

25. The ADAS of claim 24, wherein the Salmonella species is Salmonella enterica.

26. The ADAS of claim 24, wherein the Vibrio species is a Vibrio parahaemolyticus.

27. The ADAS of claim 24, wherein the Escherichia species is an enteropathogenic E. coli (EPEC).

28. The ADAS of claim 24, wherein the Yersinia species is Yersinia enterocolitica.

29. The ADAS of claim 24, wherein the Shigella species is Shigella flexneri.

30. The ADAS of any one of claims 20-29, wherein the parent bacterial cell further comprises a moiety that increases the level of the T3SS in the ADAS.

31. The ADAS of claim 30, wherein the moiety is a transcriptional activator of a nucleotide sequence encoding a component of the T3SS.

32. The ADAS of any one of claims 20-31, wherein the parent bacterial cell has been modified to reduce the level of a negative regulator of a component of the T3SS.

33. The ADAS of claim 32, wherein a chromosomal locus encoding the negative regulator has been deleted from the parent bacterial cell.

34. The ADAS of any one of claims 1-33, wherein the parent bacterial cell has been modified to reduce the level of one or more of:
(a) LPS;
(b) a metabolically non-essential protein;
(c) a toxin not associated with a T3SS;
(d) an endotoxin;
(e) a flagella; and
(f) a pillus.

35. The ADAS of any one of claims 1-34, further comprising at least one cargo, wherein the T3SS is capable of delivering the cargo to a target cell.

36. The ADAS of claim 35, wherein the delivery is to the cytoplasm of the target cell.

37. The ADAS of claim 35 or 36, wherein the cargo is a protein or a polypeptide.

38. The ADAS of any one of claims 35-37, wherein the cargo is endogenously secreted by the T3SS.

39. The ADAS of claim 38, wherein the ADAS or the parent bacterial cell has been modified to increase the level of the cargo in the ADAS.

40. The ADAS of any one of claims 35-37, wherein the cargo is not endogenously secreted by the T3SS.

41. The ADAS of claim 40, wherein the cargo is endogenously secreted by a T3SS from a species other than the ADAS T3SS species.

42. The ADAS of claim 40, wherein the cargo is endogenously secreted by a Type 4 secretion system (T4SS) or a Type 6 secretion system (T6SS).

43. The ADAS of any one of claims 35-42, wherein the cargo has been modified for delivery by the T3SS.

44. The ADAS of any one of claims 35-43, wherein the cargo is an enzyme, a DNA-modifying agent, a chromatin-remodeling agent, a gene editing agent, a nuclear targeting agent, a binding agent, an immunogenic agent, or a toxin.

45. The ADAS of claim 44, wherein the enzyme is a metabolic enzyme.

46. The ADAS of claim 44, wherein the gene editing agent is a component of a CRISPR system.

47. The ADAS of claim 44, wherein the nuclear targeting agent is a transcription factor.

48. The ADAS of claim 44, wherein the binding agent is an antibody or an antibody fragment.

49. The ADAS of claim 44, wherein the binding agent is a VHH molecule.

50. The ADAS of claim 44, wherein the immunogenic agent is an immunostimulatory agent.

51. The ADAS of claim 44, wherein the immunogenic agent is an immunosuppressive agent.

52. The ADAS of any one of claims 41-51, wherein the cargo has been modified by addition of a secretion signal.

53. The ADAS of claim 52, wherein the secretion signal is a sequence of Table 7.

54. A method for delivering a cargo to the cytoplasm of a target cell, the method comprising contacting the target cell with an ADAS of any one of claims 35-53.


SEQUENCE LISTING (54 sequences)

SEQ ID NO: 1 - DNA, 3449 bp, Artificial Sequence
  Synthetic Construct
ctcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagcagtgctcttgtgggtccgattcgccagatgataaggaacagagacgggaccaaaacgaaaaaaggcccccctttcgggaggcctcttttctggaatttggtaccgaggcatttacttatacaactcgtccataccatgagtaattccggcagccgtaacgaactccagcaggaccatgtggtcgcgtttctcgttgggatcttttgataaaacagactgcgtgctaagatagtgattgtcgggtaaaaggaccggcccgtccccaatgggcgtattttgttgatagtggtcagcaagctgaactgacccatcttccacgttgtggcgaatcttaaagttagctttaatcccgttcttctgtttgtcagcggtaatgtagacgttatgtgaattaaagttgtactccagcttgtgcccaagaatatttccatcttctttaaagtcgatccctttcaactcaatgcggttaaccaaagtgtccccttcgaacttcacttcagcgcgagttttataggtcccgtcgtctttgaacgagattgtgcgctcttgcacatagccttccggcattgcgctcttaaagaaatcgtgacgcttcatgtgatctggataacgagagaaacattgaacgccgtaagttaaagtcgtcaccagagtaggccagggcactggcaacttcccagtagtgcagataaacttaagggttaacttcccattagtagcgtccccttccccttccccacgtacgctgaacttatggccgtttacgtcaccgtctaattcgaccaagatgggtactacaccggtgaacaattcctctcccttggacatatttacctcccctatggttcgctgcaacttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccata

SEQ ID NO: 2 - DNA, 4439 bp, Artificial Sequence
  Synthetic Construct
gttgcctggctccccgtcgtgtagataactacgatacgggagggcttaccatctggccccagggctgcaataataccgcgggacccacgctcaccggctccagatttatcagcaataaaccagccagccggaagggccgagcgcagaagtggtcctgcaactttatccgcctccatccagtctattaattgttgccgggaagcgagagtaagaagttcgccagttaagagtttgcgcaacgttgttgccatagctacaggcatcgtggtgtcacgctcgtcgtttggtatggcttcattcagctccggttcccaacgatcaaggcgagttacatgatcacccatgttgtgcaaaaaagcggtaagctccttcggtcctccgatcgttgtcagaagtaagttggccgccgtgttatcactcatggttatggcagcgctgcataattcgcgtactgtcatgccgtccgtaagatgcttttctgtgactggtgagtactcaaccaagtcattctgagaatagtgtatgcggcgaccgagttgctcttgcccggcgtcaatacgggataataccgcgccacagagcagaactttaaaagtgctcatcattgggaaacgttcttcggggcgaaaactctcaagaatcttaccgctgttgaggtccagttcgatgtaacccacacgtgcacccaactgatcttcggcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatagatatttgaatgtatttagcagtgctcttgtgggtccgattcgccagatgataaggaacagagacgggaccaaaacgaaaaaaggcccccctttcgggaggcctcttttctggaatttggtaccgaggcatttaccgtaatttaatcaagcggggatcctgtttccatcttttgaaccaaatattatcttcgtttttaaatttattccacattttctcggcaatagcttcgccgtgggcaaccagcactaacggtaataatcccggattattatctatacgctgttcactttccagaaattctcttatcgtcggtaaggcacgctcactattctgacaatattcagcgtaaagaagattaacagcaataagtcctgttatttcctgcgtggatatttctttagttaattttcgtgccagttcatgtttacctttaagcgaaagaaacataacctgcatacttaataatattggattatcctgcaggtgttgtgagcgtaattcatcgcctaaacgtatagcatcatcaataccggtatgataataggtaatccacagcttagtgatccctgcggctgcgcgcgttgggtccaattttaaacactcgttaatcgtttgtaaggcctcctccaactgaccagccatgaaaagattccagccataataatatttaatatctgcagaaatgggcgaaagtaagttagcttgtttgaatagcaaactcccgacgatgtattctgagtgaatcgtattaattagccccagtaatcctaaagcttgtggattattgtggtccagctctgtcgccttaatcgcatgttctttagctttgatcatagcgttttgtttatcaaaaatccccatttgcgccatgctgaggtagcattctgccagcgcacagtaaggcgcaatgctgtttggcgacatgttaacgcattgagtcagcaatttaagcgcttgctgtaagctatagggggtgtattgatttaactcatgcttaccgcgtaagtaaaccatagtactgtcaatcgaatttagctcgctaatctgctttgtgtcccagcgaagtccgggaatacatctgagcaaaagattcgcaattttgttttgcaagagagaagcgggttggtgttctatcaacttaatgctttcctggtgcagcaggtgataacctttaacccgaactatctcaatctgtacaatattatcattaccatcgggtatcatctgcccggagatataataatcggggcgtaattgatccatgagctcaagaatatccttaacactgcggcagttcttcgtaatggtcaccggcagcacgctcaggccaaagggcgcatactgcgataatcccttcacgatagagtaatgcagactctcggattgaacctgatcctgcatctgaaaaggaagtatcgccaatgtatgagtcgtaggttgcggcgctggcggagacaccactacgaccggacgattaaaccgatagccctgtccgtacagtgtttcaatgtaacgatgctctttatcttccgacagaatacgtcgtaaggcataaatacagcgggtaagagattcttcgttaacttccgcgtcgccccatacctggtccagtaaggtgtttttactcacaatctcgccggcggcttcgagcaggatgaccagaacggcatattcttttggcggaatattgactttcttttctgagcgtagcagggagccgtccatgttgagtatgaaatcatcaaagacgaattttttattcgatacaggaacaggattaaaatgtggcataaaaaaccaacttaggaagccgaagctaattcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccagatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcttccgctactgaactactcgatgtcagagccagcgtcttgcataaaaaaacccgccgaagcgggtttttacgttattattaccaatgcttaatcagggaggcacctatctcagcgatctggcgattacgttcgtccata

SEQ ID NO: 3 - DNA, 4374 bp, Artificial Sequence
  Synthetic Construct
gatacgggagggcttaccatctggccccagggctgcaataataccgcgggacccacgctcaccggctccagatttatcagcaataaaccagccagccggaagggccgagcgcagaagtggtcctgcaactttatccgcctccatccagtctattaattgttgccgggaagcgagagtaagaagttcgccagttaagagtttgcgcaacgttgttgccatagctacaggcatcgtggtgtcacgctcgtcgtttggtatggcttcattcagctccggttcccaacgatcaaggcgagttacatgatcacccatgttgtgcaaaaaagcggtaagctccttcggtcctccgatcgttgtcagaagtaagttggccgccgtgttatcactcatggttatggcagcgctgcataattcgcgtactgtcatgccgtccgtaagatgcttttctgtgactggtgagtactcaaccaagtcattctgagaatagtgtatgcggcgaccgagttgctcttgcccggcgtcaatacgggataataccgcgccacagagcagaactttaaaagtgctcatcattgggaaacgttcttcggggcgaaaactctcaagaatcttaccgctgttgaggtccagttcgatgtaacccacacgtgcacccaactgatcttcggcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatagatatttgaatgtatttagcagtgctcttgtgggtccgattcgccagatgataaggaacagagacgggaccaaaacgaaaaaaggcccccctttcgggaggcctcttttctggaatttggtaccgaggcatttaccgtaatttaatcaagcggggatcctgtttccatcttttgaaccaaatattatcttcgtttttaaatttattccacattttctcggcaatagcttcgccgtgggcaaccagcactaacggtaataatcccggattattatctatacgctgttcactttccagaaattctcttatcgtcggtaaggcacgctcactattctgacaatattcagcgtaaagaagattaacagcaataagtcctgttatttcctgcgtggatatttctttagttaattttcgtgccagttcatgtttacctttaagcgaaagaaacataacctgcatacttaataatattggattatcctgcaggtgttgtgagcgtaattcatcgcctaaacgtatagcatcatcaataccggtatgataataggtaatccacagcttagtgatccctgcggctgcgcgcgttgggtccaattttaaacactcgttaatcgtttgtaaggcctcctccaactgaccagccatgaaaagattccagccataataatatttaatatctgcagaaatgggcgaaagtaagttagcttgtttgaatagcaaactcccgacgatgtattctgagtgaatcgtattaattagccccagtaatcctaaagcttgtggattattgtggtccagctctgtcgccttaatcgcatgttctttagctttgatcatagcgttttgtttatcaaaaatccccatttgcgccatgctgaggtagcattctgccagcgcacagtaaggcgcaatgctgtttggcgacatgttaacgcattgagtcagcaatttaagcgcttgctgtaagctatagggggtgtattgatttaactcatgcttaccgcgtaagtaaaccatagtactgtcaatcgaatttagctcgctaatctgctttgtgtcccagcgaagtccgggaatacatctgagcaaaagattcgcaattttgttttgcaagagagaagcgggttggtgttctatcaacttaatgctttcctggtgcagcaggtgataacctttaacccgaactatctcaatctgtacaatattatcattaccatcgggtatcatctgcccggagatataataatcggggcgtaattgatccatgagctcaagaatatccttaacactgcggcagttcttcgtaatggtcaccggcagcacgctcaggccaaagggcgcatactgcgataatcccttcacgatagagtaatgcagactctcggattgaacctgatcctgcatctgaaaaggaagtatcgccaatgtatgagtcgtaggttgcggcgctggcggagacaccactacgaccggacgattaaaccgatagccctgtccgtacagtgtttcaatgtaacgatgctctttatcttccgacagaatacgtcgtaaggcataaatacagcgggtaagagattcttcgttaacttccgcgtcgccccatacctggtccagtaaggtgtttttactcacaatctcgccggcggcttcgagcaggatgaccagaacggcatattcttttggcggaatattgactttcttttctgagcgtagcagggagccgtccatgttgagtatgaaatcatcaaagacgaattttttattcgatacaggaacaggattaaaatgtggcataaaaaaccaacttaggaagccgaagctaattcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattaccaatgcttaatcagggaggcacctatctcagcgatctggcgattacgttcgtccatagttgcctggctccccgtcgtgtagataactac

SEQ ID NO: 4 - DNA, 5778 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgtttacaattaatcatccggctcgcattatgtgtggaattgtgagcggataacaatttcacacaggaaacagaccatgaatttagatggtgttagaccatactgtagaatagtcaataaaaagaatgaaagcatatcagatattgcatttgcacatataataaaaagggtaaaaaattcatcatgtactcacccaaaagcagcattggtttttttaggagagaaaggtttttgtgatagcaatgatgttctatctattatgggacaacaaataccaagagtatttaagaacaagatgttatatgattatgtttttaaaaatgaaaaaagtaaaaatgattttctaaaaatggctgaatcatggctaccacagagtgaaccaatagtaataaataatgatgatgacgcattgaatgctgctgcttatttttctgtaaaaaaagcgaaaataaaaacagtaaacgatactgattttaaagagtataataaggtttatattcttgggcacggtagtcctggttctcatcaattaggccttggttcggaacttattgatgtacaaacaatcatttcaagaatgaaagactgtggtattctaaatgtgaaagatatccgttttacttcatgcggctccgctgataaagtggctcctaaaaattttaacaatgcccctgctgaaagtctttcttgtatccttaactctctgcctttttttaaggaaaaagaatctttgctagagcagataaaaaaacaccttgaaaacgatgagtcattgagtgatggtctaaaaatatccggctatcatggatatggagttcactatggtcaagagctttttccctactcacattatcgttcaacttcaattcctgctgatccggagcatacagtaaaaagaagctctcagaaaaagacttttattattaataaagaactggatcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaaaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 5 - DNA, 32767 bp, Artificial Sequence
  Synthetic Construct
ttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaggatccctatattaccctgttatccctagcgtaactatcgatctcgagtacggccccaaggtccaaacggtgaatgcagaaggtgtagagtctcgcagatttctgacatcatagagcctaacattagatgcataacgctacagattgtagaatctctacaggtgaaatatacactggttggcgtttatgtcgcaaaacatgtcattcaggttcatctcaccaataaggatatgagtgaagtggaggataagtgagtctgctgtcagagttttctggtgtatgtcagtaactgcgaaccatgtctgcttgatatgggagcttatggaggctctctgtacaaggttagggagctaacaaagcagggataaaaagtccgcctgatgcaggcacggttcgttaaatcttttgccattggtaacaaaatggtgcgatgaatgttcaggctatctttatcttgatggtggttcagctgtttggtaaagaaatcgctgtaacaatagaagaactgcaggcagtactggtcccacatcgtttgctcagtcaactggtgaagttccgtagcgcgtaaattaatgctttgcatgaaacgttactggagtggatgaaagaatacctgcatatatcattactgcgttggattaccagtacaacagattgaatgaactggacatgttgatagacggcattgaaaagcagcttataagcatagtgcggcagagttaattggagagcacttcgccttctgagtattaagtaagcatggtggtacctgcggatactatctattcactgtgctcggtcggcagccttactgaccaaggagttgggggagttgataacaaagttgaaaaaacagctctcaaccaaggtggttattgtaacaatagcaaataagctggtgtgacctgtatcacaataaccgattatggacgtaaatacgacaaaaatatgtcagtatcaggcggtattaattgctgatgctgatggacattaactcttaataaaagggtggatgctgaaaggttgttatggtgacggacgtgatgacgacggaaaagagtatgaggttttggatttaaagttcgttcggggaataaggacccggtcagctaattctcatagtagttcgcattgctggatgcaatactgtgctgcaattactcgtcgatgtcggaacaacggaagccttgcaaacggggatgtattcatacaggtgttatgttatatggggttttattgcagggggctataccaagagaatatatgatatctcatcagactgatgtgagggtaaatgagaatagagtaaacgaacaaggttgttttttggcgaggaagcagatgtatgacaattcttgtggagctgccagtttactctgtgctgcaaaggaattaggggttgataaaataccacaatacaaagggtctatgtccgaaatgacaaggaagagttctcttgatttggataatcgttgtgagcgggatttatacttaataactagtggtaattataatccgagaatacacaaagataatatcgcggatgccggttattctatgcctgataagatagtcatggctacgaggcttcttggattgaatgcttatgtcgttgaagaaagtaacattttttcgcaagtaattagttttatttatcccgatgcaagggacttattaattggcatgggatgtaatatagtacatcaacgagatgtgttatcttctaaccaaagagttttggaagctgttgctgtatcctttattggagttcctgtcggcctccactgggtgttgtgtagacctgatggaagctatatggatccagctgtaggagaaaattattcttgtttttcaactatggaattaggtgcaagaagaagtaattctaactttataggatatactaaaataggtatttccatagttataactaatgaggctttgtaaattgtcgttttttatctgtgaaggagcttgaatattttggggtggggggcataacaaatgatattactgagtacaaatttagctatattgggcggattagttgtgtgggaagacgaaatgtttctcttgaaataagtgaactccctcaaggaatgattgtgtgcttaaatatttaagtgctggtaagtttgagaaggctgacatattttgcagagcaaacagtgattgacttggatgatatttttattttagtcactcgttgtaattggagtatgctttattttatttgaaaatagatgtattttatacccgcaatttaaactgtaagccctatgaggggtatggatattataagttactctttgaccaagcataacttttggggttagaaaaataaatctcttgttaataattaatatgattttaaaatcaaacttgtttataattataaaataatgtaagtcggtcattttatgggaagattaggagaataaaacagtaattatatttgagaaatctgctaatatattcttaaagcttttttgatactgtgaacgaagtctgttaatggtggattttttgtttatcaccatgtaaaaaaaattgagtagtaaaagcgttgactgctaatcctcgctgtttttattgaaacgattaaaaatacgctgaatattttcggccagtcactcatattcacataaaattttgaccatcatgccgcatcccttagttgcacaattcattttcaatgatatgaatattactaatttcaccagataattttctcgtggtcagcagggtatgagttttcaccgtaattttaaggaaacacagtgcgtggggattgaataacagatagccgacggaattaccccaccggctgaaagtcaagttcaacgaagatttttattatctgaattgggcagtttacatcagtgttcgatgtttatgaagacgatagatggcgagaaattatatcccgaatagcttcatctatttctctctgaactgttttgtttattttagataatgtaaatattagagttctgtttgttttattctcttttctttttatgtaagtccgtttgtctttagaaataaataacgagtcaacttttagcgaagggttttgctttctgtttttgcttttctttatgatgtttaaaatttcttttttatatatgtccgggggcaaatttgtatcaaggtcttttatttcttcctttaatattggtagtgtagaactaagagattcattagccttttcaagtcctttataataattgaataatatcttgtagtcattaaagttaagttctgacgcgattggaaatagagatattatttcctgtggaacgcttgctgcctgaaaggcacgagtgactttcgcgcgagacagattctcttttttggctatgtctttataggacatccctgatactttcagaaaattaagaccaataccaagttctcggatgctatgctcttttgctgtttgtatatcgtttgctagttttctggcatcaagagtagatatatattcttttgaatatagaacttcaagatctgctcctgcatatattgcagatgctcttctacgagtgccatccagaatttcaattctaccatcaatctcccttcctattacagggaagaattgttgtagctttatagtttttatgatatcggctaatgattcttctgttagggattcttgatcgcgtacattcgttttttggttgacgaaggttaaatcttggatccttttatgaggaatcttggctttgataaaggaaactttattaccagaatggagtgtaaactctttcttttggccttcctttatacttaacaagtcgttgcacaaatccaccatatcacaccctgtttattcatattgatttatattgacaaaagttaaatgcagtgtgagcaagattgtatgtaactgttgatgatagtcaacggtagtcttaaaaattactattttaatagctggattgatgcaatttatctttattggataaaagaataatattcttgtggagctctgatgtgagagctccatagatgaaaaaatgcttatcaattactatattgattttgtactatttttgactacagaaataagatctccaatagtaataatgtgttctaatgtagattcatctatacgtagattgaactcaaaactaatcatatccagcatttcaatcagagaaagagaatcggcagaaagatcttcaataaatttagtttcttcagatagtttgctgtacgctataccatagcaaaaggccactattgataatattttttcttttatcatattttaatccttattgatattctttaatacttttgatagggaagtggttacatcttttgctgctttatatatattattgttttcagctgtaatatcattgttattttttaaatcagatatatcatctgtaagtaattctgcagaggtatcgtcaatttttgacaataccttgctgagggcgctagatacttcttttgccttttcatagatcgcatgatttttatcaattgtatctgcgttggtttctgttacgtagtttcttatatttgcatctgtaacttttaatttgttgtttattctatgttctgttgtggattttgtaatgtcctttgatttttcctgtaactctcgttcttgtgttcctgatttagttccaatgagagatataagattacttagggagtttgttactttttttgaagcttcaaatatcgtttcccgtgttaaatatgtgtctccatttcttgataaaaaatcagtctgaagtacttttgaatcagtgtttgagttgaatttattgatttgcgaagctactctatgagaaagagaagcgctacttttagttgtatctgatgagttatcattactgcttgtcactccttccgggtggagagggggggtggttgaagagttctgtatgttcagaggcctgtgtccccgagaaagagagttatttgcagatctttcttgactactcgtagttaacaggtctgaacctattccatccgaattatctggagtatggttgaaagccttttctaaagctgaattatttgcaaatgtctggacagtatctgatatagagtgtggtagaacggaagttactttttgaactagtgctctttgtggatttagtaatgagtttccgttacgggataatagatcgtttgtctgagaagtaggagactgtgcatcattttctgggttctcgtgatagctattgtcatatactctgttgtcaaaaactctattatcgtaagttctattatcattatttatatgatagtgaattacaggttgggatgttttctcattggctccgggggttggtccgccatctggcatatcttcgggaggtgtagggaggccatcttggataggatgcgaatcttttatatcttctaaaatatcattaaggacttttctcaacagctcaggaaacacggctactgtatttaagtctcttgacagtttacctataccaaatcctttactatcaatagtatttttacgttgattgcgcaaactatcaaaggcaagttttgcctgtgcgtcaactaaatcatctagtttgagagatgaaacagtttcattacttaactttaattcagcaagaatattattcttgataaatgatgatacttcagtattgaggagtttttgtcccaatgataaagcttcaccagatgtgtgttgttgtgctaatagtgcaagtctgtgctcaatgttttgtcgtaattgatggagtttcttattctttgccgaccatggtccaaaggcagaatcttgggataatttatcttttataattagttccgaatactgtgcgacaattttttgaagtcccttttcgttttcaaaattaagaccattttgatgtactaaggagtacaaaacatttgaaaattcgagaatctcgcttttggcatacgcattcattgagttggaataaatgcttcttaacacatcttctttccctgaggaagaaaataaaaaatcaagacatttctgattaaaagacgttgcaaagttttctttttcagacactgatgctggtgtttttagagaagtttgcgaactatggatatcggatattttgctttttaactcgctgtattctgttgatgatggtgaagttgccttatataagaatgttggcgcttgagtattatttacattatgcatggcgcacctcagaaatggagaaaaagtttatctgtatctgtacatgagcttattgtactactcaaaacctttactaaattatcaaaaatactattggcattactgtatttttgaactaaagtttgaagattatttttcattgtttcatcttcggcagagaatccggcattccatgcctgatattttgcattatctagcacaacctcgccatttccacctagattatctaagctttttaacatattgtctattggggtcatgtttatactgacaacatatcccccgtttttttgagataccttgccgattgttccacctaattctgtaagccatttatttgcttgttcctgactaacagtattatttgctggatatagcggtttatctttatatttttccttgagttcttccaatgcctttttaagcgagttgacttgtaatttcacggagtttccgtcgttacctccgggagagatccagccggcaagactggaaagaacagcgctaaaatcttgatacatttgagtatatgaactaacggcatgttcatatactttcagatactgttcattaatatcattgatggagtttgcaattttagcccacagttctctatgagatatcatttgatctccatcaagctcggcttctttcggagctgaatgtaataattctcttgcgtctttattaattggatattcgtgcctggaaagaatatctaatagttgagcggatttatttacatccatgctaatctgagatgaatgtaatgctatttcttctagcgatgttttagtcaacgttttttgtgaaagctctttctttaatgcctgatttgttgttctgatattatgaagggtgtcgttgagcatagtaagggaacttacaggatgagaactggtcgttgtttttatatcagaattaactgtttcggttgatgaaccgttggtattgtttggactgaatgatgaggtggaaatactattagtcagagttgttatattcatggttatttccttacaggaagagccatatattattatggctcttggatatattaagctcgaatgttaccagcaatctgactggctgtcgaattctttgattggttgatgctgtcaattatattcaataatttttgtattaattgatttgtcgcttgggatgcttctttagatacttgggatgcttcctctgcttgtttactgctggcctgactgattagttgttcttcttcttcaagagcagatgcataacgccctcctgatgtggatatattaccagcaacggctgatgtttccattgttgcacggccaatgttttgtttttgctgcgcagaaagggtattgagctcgtaagttcttctctgggtgtcaattttatcctgcaaagaaatatcgggagaactaagagaagttttatgttcagttgacagggatatattatctggcgccagtttatttttacctaaaaattttgtgctagagttagtttgtggtgaggtgatatttgtatctatttgtttatttaaccctaatttagaacctgcaagctctttttcaagagattgagcagtggcaaggttctttcttaaggctcctttttggtcgctaatccctgaatgcgtttttttggcaccgatacccgttatacctacttgtgtgactgctccagtaatgcttgatgatagggctgccaggccttgccgaacaatgttctctgcagctgattttgtagcatcgaacgcaatcaatgacaattgagagcccaatttagtttctgcagtgcggagagcagaaagtagaacggctacactaataattaaagaaacagcattagaagaaagactggaaatatccagagtgttctctggagtaagggtgtgactcatctctgaaagatctttcatctttttgtcagtaaggcgagcaatgatttctgaatcatgctgaacctgctctgataactttaaaagttgatcatcatttaatgtggttgttaatacgggatttttaccgacattaagtggaatatgcgctgcaatctgctgataattttgtgatttttgtgtttcggaagaactttgagtttgttttgtggatatatctgtatataaaatctgggttggttttgtgttttgaatttccataacattctccttatttgtatcaagcagtagtttgttgcaaaattgcttttgcaacatcagttctattagcctgagagttggacattgaggctaatagatctgcaattacttcctgcaattggccgaatttttctattgcttcactgatatattttgacagttgttcaacttgatatttcgataatgtcaggtctgctagatttgtcgacgcgctgttctggaaaacagcagaagcgacactttcctcccgcttgtgtggcagagttaacactttctcctaataaaactgcttggtttaaatgggtggaaataatttgtttggatattacttcatcacccgctgcaccaagaaatttatttaatctggcaacagcattagtgattaaatcgtccagttgagaagaaaaattcttgagaaactttggtataaggtctgtgagggttttacctattattttgccaatattttctgcaagttttgctgctgcctgtttaccaacagtggctacgagaacgactgctgcaactaggacaagagcgcctgcgattgcccccagaatagagccaatcattttggcttttttcgagtcgacgcccaagccttcgagcatttttgtaaatgcatctgaaaggagtttgattaagggttcaatgactgctttcatgatcggattcagggcttgttccatgaaggaattgccggtcgctgcttgtactatagcatccgtaaccataagagctaaaccaacagctgccagtgctagagaggctcctccagaaaaagctgctgcaacaacactaacgatagttaaaagtgccccaagtattttcccaacacaacccattactctgttgagttcttctgctttacgtacttcagcagcatactcatcagattttctctccatttcagtttttcttgattcttggagagactggaatagagccagatcattttttaaagattcttcattattttttccaactagttgaataaaggttgccatcaattgagtaacactggcaagtccggttaatgatttctgctgggttgatagctgttcagcagatgctgtgtttgaaaatgcagaaaaagagtctatttctttttcgagttgcattgatttatctgtaagtttgctatgaattgacagggttttctgctcaatcaatgtcctgtctttaactgctgcgtcttttttgatagtgagttgtatttcttcccggcttaattttttcttttctggtgactctgggtcgagttcggataatcttgtttgaatttggttaattttattttctaggtcttttattttagaatctgcgttttttagtttattaatttgtttttcatagtctctggttagtccttcagtttcagatagaagagtgttaattttatcggagaattctaggtttttttgctgtcttgcctgttgctgggacttccaagcagtaattttatttgttaatgcagttaaagatttttcaccgagtatttgaataaggtttccaattaaaagcgttagttgggaacttgcatttaatgactttggtgctttaagttcagggattaatatatttgaagttgagtgtgcattagttgtattaatattttggttagcagtaagatcagcaattgtaagagaaaataatttgttagctgcatcatttgcagcttggatagtattgtctccaagctcagtggaagccaatattttggcaagaggaaaaccagtggttgtggtgcttacattatgcataataattactccttgatatcctgaattgcgtccaagtatgattgtgcttttatttttaatttttcatcattgctgtgttgaattacgagttcgaagcactctttagcttttaagggggctttcaaccgaagctggcattgtccagtatggaatactggtgtatagtcattttttcctaatgcaaaagcgacagcataaaggtctgctgcttgttggaactgttcttttatctgataaatagctgcgagtcccataatgtagtctacattgtaaaagtcgtatatacataaaaacctgaagaaaacttcagcttcctctattcttcctttgttgtaaaagtcataagcatatgaataaatgtcatccatcatatcatcaggaattgcattaatatctttcagtgtagcgccagagttaattgcatcaattactgcagtagagatgctttcattttcggtgatatttaaagacataaggtctcctttttgtctgctttactttaataagtataagatttaatataaaagatttaatttgtattgctttgacggtatacagcctttagaacctctgcgcttatttctatttgtaaaacttttgttgattcattaaatattttatcgtagacggaactggatactagatttgtaagatccttcatttgtgtgttaaacaaatccatgtcactgctatagcgttttaaaatctttgctctgaggccagatgaggcaatctcgtttagtgctgtatgactgatagatgtttgtgctgatttttcatcaagttttatatccgggaattttttttcaatagctgcaaatatgttttttttgttatctgaggtcaaaacaactccattgtgtatggccatttcatctatacttctgtttatcattgaagagacataagtggttcgctcttggctcatccaacaaaatagttgattccccgaattactatcttttattttgtctatgacgctgtccctgattttatttatattcaccgggcatccaaaattcttctgaggaaagcgttgttttaagttaaaaacagataatattttctttacaagcttattgcgacttaacgaatcaaatgatgggcgaggaatcgcgaattcaacctgtggaagtattttgtttagaatttgcatgataccccctatatgttagttcacttctgaagtgatgtttgcaatggttagtttaatacaaaataaaaattcatacaaagattcaatgagttcatcaggtgaaagatattttttgtagtgtttcttggtcaaaacacattgatgagtatcagggagaagacagatagaagaaccataatctctactcaaagagtagttaaggatgagtatgtgttctggattaataatattttctgtaaaaagttcatgttcattaagaagtccggtcaaaaagatgtattcatcattatctgttatacaaacaggtatttgttcttcgagcaatataaaggcttgatttttatcgttaaaatcaagctttgcacctgttatttcataaagtttatcatatagtttgcgacacatttctatggccttctatatattagaatgagagttattcaataaaagcttattgatctgtaataagtcgtgaatgtaaagtttggttccaagatctggcgatttaagagaattgtaataatcaataatttttattgatttttcgatgatttcttcttttttatctgttgcttcggtttttaattcgtttaatacacgagtcatattgctgggtgctgattttactaagtatgagtggcaagcgttgatgaccgcatcaaagtcttctgaagatacattttcagtaggatgtttgtaaagtgaaattgataaaggcttgaatgtttttggtgtgtggataaggttaattttattgaaacttttaagatagctgcgtcgtaattgagtcaatctggaaagatcaaaatcagattcaatatacttcattatttcgttataagactgattgagactttcaatgcgttgcctaactttatttgcatacgcgtatacatcgttaggtgtagcaaccaatttaacgtcagggagtggaaagtatatttcagaatttcctgctttaagaacattaagtgccataccagcacagttttcagatttactgattaatttataatttgccaattgtctgaaagcatctgctttctttttgcatataaattttgacatgtttgtttcatcaaggccaaaaaggttatggctgattttaccatcctttgttttattatcacctgataatggaatgtatactttatcagcactgaccccccaataaccataggggtcttttttttgattagctctgggaaaaaatatttttttgttgttattttgatcttgttttgaatttaataagtttctaacctcttggcctgcatcaagtctttcggctgtttggtctgaaatcatatttagtttgtcttgcttataggtatcgacactgtaacctgagcgtttttcaaaaaggtagtttatttctgtatcttttgtaacattcccgccgggataccagctgagataggtatgagttggttcctgtagtaaattgttttttatactgatactagtatgccctaaatgggttatgcctctatttccccagcaacgaaaaactaaatgtctttcaagatttttggctgtaccgatgccatgaaaacgctctgcggtattcaacattaatttgatgtctctacctgtgaaagagtgttgaattgagtttttagctgcttcataggcggcaaacgcactttctgcagatttttcgctagctaatgtattgaggaaaagcaataaggatttttgattctctagtcgataattttttagttgttcatttaatatctgtgaatttatctttgcattaatagtgcttaatgttctgaaaaagagatcctttaggttgaatttctgtgctatcaatataggtccatgctctgtatcttcaacgcgtataggcccttttgtattgcttgcgtcaatgaaattgctaattttgaggatcatactttattaactctccattacttggtgattgtggggatgtaaaatgtatttggtatataatatattcaattaataaatttagaaacttgagcctgttaacataatcaaattttcttttgctgtacataatatgtacctcgtgagcatatgtagtgctcgtttcatcatgaaatcccacaagataaagtgcctgatgtatcaggctcggagtgttatagaaaaagagagaaccctgttgaataagtatcttttttgttgtaaaacgaggtacaaaactgcaatcaatgccccatttatgactgaggataattaaatgcacataactaatttgggattgcatcaggtttcatttcaaagcggagattcctataaaggcgcagaagaaaccggaaagcacaaaggtgtaagcgtgatttcatatcaaagagttaaaaatggagaaagaaataaggggatcgaagcgcttaatcgattatatttacagaaccaaacaagcttaacaggaaaaagccttttgtttgcgcgtgacagagctgaagttttttgcgaggcaataaagttggcaggtggtgatacgtcaaaaattaaagccatgatggaacgattagatacctataaacttggtgaagttaataaaagacatattaatgagcttaataaagtaataagtgaagagataagagcacagctaggcattaaaaataaaaaggaattgcaaacgaaaataaaacaaatatttactgattatttaaataataagaattggggaccggtcgacaagaatattagtcatcatgggaaaaattatggttttcaattaactcctgcctctcatatgaagatagggaataaaaatatatttgtcaaagagtataatggaaaaggaatttgttgtgcttctacgagagagagtgatcatatcgcgaatatgtggctttcaaaagtggtggatgatgaggggaaggaaattttttcgggtatcagacatggagttatatcagcctatggattgaaaaaaaactcatctgaaagagctgttgctgctcgtaataaagctgaggagttagtaagcgcagcattatatagcagaccagagttattatcacaggctttgtctggtaaaacagtagatttaaagattgtttcgacttctcttctgacgccgaccagtttaaccgggggagaggagagtatgttaaaagatcaggtgaacgcattaaaagggttaaactctaaaaggggggagccaacgaaattattaattcggaatagtgatggtcttctgaaagaagtaagcgttaatctaaaagtggtgacatttaactttggtgtaaatgaattagcgctaaaaatgggcttaggctggaggaatgttgacaagcttaatgatgaatcaatatgttctttgttgggggataatttcctcaaaaatggtgtgattggaggttgggctgcagaagcaatagaaaaaaatccaccatgtaagaatgatgtgatatatctggctaaccagataaaagagattgtaaccaaaaagttacaaaaaaatgataatggagagccgtataaattgtcacaaagaatgactctattggcttatactataggtgctgtaccttgctggaattgtaagagtgggaaggacagaacaggtatgcaggatgctgaaattaaaagagaaataataagaaaacatgaaacaggtcagttttctcaattaaatagtaaattatcctcagaagagaaaagattattttctactattctaatgaatagtggtaatatggaaatccaagagatgaatactggtgtgcccggaaataaagtcatgaaaaaattgccgctatcttcgttagagctatcttattctgaaagaataggggacccaaaaatatggaatatggtgaaagggtattcgtcatttgtataagaggaatatatggaagatttagcagatgttatttgccgagccttgggtatccctttaattgatatcgatgatcaagcaatcatgcttgatgatgatgtgctaatatatattgaaaaagaaggagattcaataaatttattatgccctttttgtgcattgcctgaaaatataaatgatcttatatatgcgttgagcctaaattactcagaaaagatatgcttagctactgatgatgaaggcggaaatctaattgcgcgattagatcttaccgggataaatgaattcgaggatgtatatgttaatacagaatattatatttcgcgtgtgcgttggctgaaggatgaatttgcgcgaagaatgaaggggtattaatgtcccgtttcgtatttattttgctgtgttttatcccatatttagggagagctgattgttgggataaggctggtgaaaggtacaatattccatcaagtttgttaaaagcgattgcggaaaaagaatccggatttaataaatctgcagtcaatgttaataacaatggaagtaaagattatggtataatgcaaataaatgactttcattctaaaagacttagagaaatgggatattctgaggaaatgctaattagtcatccatgtctttctgtacattatgcagcaaagttattaaatgagtttatgatgatgtatggaagagggtgggaggctgttggagcctataatgcaggaacgtctccgaaaaagaaaaaagaaagattaaagtacgcagaggatatctatagacgctatttgcggatagcagcagagagcaagcagaataatcgaaggatataagaggatttatgtctgaggcaaagaactcaaatcttgcaccattcaggttattagtgaaactcacaaacggtgtcggggatgagtttccgttatattatgggaataacttaattgttctcggacggacgatagaaacacttgagtttggaaacgacaatttcccagaaaatattattcctgtaacagatagtaaatcggatggtataatttatttgaccataagcaaagataatatctgtcaatttagtgatgaaaaaggtgagcaaattgatatcaattcacaatttaattcatttgaatatgacgggatttcttttcaccttaaaaatatgagggaggataaatcgagaggccatatactaaacggaatgtataagaaccactcggtgttttttttctttgctgttattgttgtgttaattataattttttccctctctttaaagaaagatgaagtaaaagaaattgctgaaattatagatgataagagatatggaatagtcaacactggacagtgcaattatattcttgccgaaacacaaaatgatgcggtatgggcgtctgtagccttaaataaaaccggatttaccaaatgcagatatattctggtgtcaaacaaagaaataaatagaattcaacaatatatcaatcaacgattcccatttatcaatttgtatgtgttgaatttagtgagtgataaagcggaattactggtgtttttaagtaaagaaagaaatagttcaaaagataccgaacttgataaattgaaaaatgctctgattgttgaatttccgtatatcaaaaatattaaattcaactatcttagcgatcataatgcaaggggggatgctaaaggtattttcacaaaggtaaatgtccagtataaagagatttgtgaaaataataaagttacatattctgtcagagaagaactgacagatgaaaagcttgagctcattaatagactgatttctgaacataaaaatatatatggagatcagtatattgagttttctgtacttttgatagatgatgattttaaaggtaaatcatatcttaacagcaaagacagttatgtaatgttgaatgataaacactggttttttttagataaaaataagtgaggataaaatgagtgttacagtaccgaatgatgattggacattgagttcattatctgaaacttttgatgatggaactcaaacattacaaggtgaactaacattggcactagataaattagctaaaaatccttcgaatccacagttgctggctgaataccaaagtaaattatctgaatatacattatataggaacgcgcaatccaatacagtgaaagtgattaaggatgttgatgctgcaattattcaaaacttcagataatagggagcattcatgaattacatttatccagtcaatcaggttgatattatcaaagccagtgattttcaatctcaagagatatcaagtctggaagacgtcgtgtcggctaaatatagtgatattaagatggatacagatattcaagtatcacaaataatggagatggtaagcaatccagaatcattaaacccagaatctttggccaagttacagacgacgctctcaaattattcaataggagtatcattagctggcacgttagcaagaaaaacagtttcggctgttgaaactttattaaagtcttaatttatatgattaggtataaaggttttattttattcttgttgctgatgttgattggatgtgagcaacgtgaagagttaatttctaatttatctcaaagacaggcaaatgaaataatatctgtgctagaacgccataatattactgctagaaaagttgatggaggtaaacaggggatctcggtacaagtcgaaaaggggacatttgcatcggcagttgatttgatgcgcatgtacgatttgccaaatccggagagagttgatatctcacaaatgtttcctacagattcattagtgtcttctccaagagctgaaaaggcccgtttatatagtgctattgagcaacggctggaacagtctttagtttctattggtggtgttatttcggcaaaaatacatgttagctatgatcttgaagaaaaaaatatatcttcaaaaccgatgcatatatcagtaatcgctatatatgactcaccgaaagagtctgaactattagttagtaatattaagcgatttttgaaaaacaccttttctgatgttaagtatgaaaatatatctgtcatattaactccgaaagaagaatatgtttatacaaatgtacaacctgttaaggaagttaaatcggaatttttaacaaatgaagtaatatatttatttctcgggatggctgtactagttgtcattcttttggtatgggcattcaaaacagggtggttcaagagaaacaaaatatgataagaatggatggaatttataaaaaatatctttcaataatttttgatccagcgttctatataaatagaaatcggttgaatttgccttctgaactgttagaaaatggcgtaatcagaagtgagattaataatctcataattaataaatatgatctaaattgcgatattgaacctttaagcggggtaaccgctatgtttgttgccaactggaatttacttccagctgttgcttattttatcggtagtcaagagtctcggttaattaatcatagtgagatggttattagttattatgggggcaaaatcagtaagcaaggggaggccgctattcgaagtggtttttggcatcttattgcgtggaaggaaaatatatctgttggtatatatgaaagaataaacttattatttaatccaattgctttggagggaaattatacgcctgtagaaagaaatttgtcccgattaaatgaaggtatgcaatatgcaaaaaggcactttaccggtatccagacatcatgcctatgatggtgttgttataaagagaattgaaaaggaattatgtaaaaccataaaagatagagacactgagagtaagaagaaagcaatttgtgttataaaggaggctacgaaaaaagctgaatcactgcgtatagatgctgtatgtgatggttatcaaattggtatacagactgcttttgagcatattattgactatatttgtgaatggaagttaaagcaaaatgaaaatagaagaaatattgaggactatataactagtttgctttcagaaaatttacatgatgagaggatcattagcactttattggagcagtggcttagttcgttgcgtaatacagtaacagaattaaaagtggttttgcccaagtgtaatttagcactgaggaaaaagttagaacttgatctccataaatatcgctcagatgtcaagattattttaaaatattctgaagggaataattatatattctgtagtggtaatcaagtagtggagtttagtcctcaggatgtaatatcaggtgttaaaatagaacttgcggaaaaattaacaaaaaatgataaaaaatatttcaaagagttagcacataagaaactgagacaaattgcagaagacttgttaaaagagaatcctgttaatgattaatcaaataaatgcaagcaatgctctgcagcaaagattaaatagtgaagaagttgttaatttaaatgagagattatcatcttcgcaatcatttgatgaggatataatctatgaaataatgcaatatttctcacagagtgaattaaattccatagataatgatgaactgcacaataagattgaacaattattcaattcacgttttccttatcttacagctgcacaaaaaagttcacttcttaataaactcatagatgcaaatcaatatgtcgatttgcatgagggattttatgcatcattgtccatatataacaatattgatttctatattaagactacaaccttcgattcattaattagtgtctttgaagcagggagagaggcagatgattcgacatggtagtaataagttgaaaatatttattttaagtatattgctattaacactgagtgggtgtgctttaaagtcatcatctaattctgaaaaagaatggcatattgttcctgtaagtaaggattatttttctattccaaatgatttattatggtcgtttaatacaaccaataaaagtataaatgtttactctaaatgtattagtggtaaggcggtttatagttttaatgcaggtaaattcatgggcaactttaatgttaaggaagtagatgggtgcttcatggatgcacaaaagatagctatagataaactattttctatgctgaaagacggggttgttttaaaaggtaataagataaatgataccatccttatagagaaggatggggaagttaaattaaaattaattcgagggatataattgtattgtgagtaaatataaaggtctaaatacaagtaatatgttttacatttactctagtggacatgaaccagttaacgttgagcttgtaaaagataaagaacgtaacataattgagctggctccagcatggaagggtttttttttgtccgaaatcaaaatataaaattcagtgataatgtgaactatcattatcgctttaacattaatagttgtgcaaaatttcttgctttttgggactatttcagtggtgctctagtagagcatagccatgctgaaaaatgtattcatttctatcatgagaatgatcttcgggatagttgtaataccgaatcgatgcttgataagttaatgttaagattcatcttttcatctgaccagaatgtaagtaatgcactggctatgatacgaatgactgagtcatatcatttggttctttatcttctgagaactattgagaaagaaaaggaggtaagaataaagagtctgacagagcattatggtgtgtcggaggcttattttagatctttatgtagaaaagctttaggggcaaaagtcaaagaacaactgaacacttggcggttagtaaatggtttattagatgtatttttgcacaatcaaacaatcacttcagctgccatgaataatgggtatgcatcaacatcccatttttcaaatgagataaaaacacgactaggcttttctgcaagagaattatcgaatattacttttttagtgaaaaaaataaatgaaaaaatttaatattaaatctttgactctcttgattgtattgttaccattgatagttaatgctaataatattgactctcatttattagaacaaaatgacatagcgaaatatgttgctcaaagtgatacggtaggatctttttttgaacgtttttctgcgttattaaattatcctattgttgttagcaaacaagctgcaaaaaagagaatatctggggagtttgatttatctaatccagaagaaatgcttgaaaaactgacattacttgtgggactcatatggtataaagatggaaatgcattatacatatatgactctggagaattaataagcaaagttattttgctggagaatatatcacttaattatcttattcagtatctgaaggatgctaacctttatgatcacagatatcctataagaggaaacattagtgataaaacattctatatatctggtcctccggcattggttgaactggtagctaatacagcaactttgcttgataagcaggtttcttctattgggacagataaagttaattttggggttataaaattaaaaaatacatttgttagtgacagaacttataatatgagaggggaggatattgtcatcccaggggtggctactgttgtagaacgtttattaaataatggtaaggctctgagtaacagacaagcccaaaatgatccgatgccaccctttaatattacacagaaagtatctgaggatagtaatgacttttcatttagttcagttaccaatagttctatattagaggatgtatcacttattgcatatccagaaaccaattctatattagtcaaaggtaatgatcagcaaatacagattattcgcgatattatcactcagttagatatagccaaacgacatatagaattatcattatggataatcgatattgataaatctgaattaaataaccttggtgttaattggcaaggaacggcttcatttggtgattcgtttggtgcgtcatttaatatgagctcatcagcatcgataagcactttggacggcaataagtttattgcttcagttatggcattaaatcagaaaaagaaagcaaatgtagtttcaaggccggtgattttgacacaagagaatattccggctatatttgataataacaggactttttatgtgtcattagttggtgagcgcaattcttcgttggagcatgttacgtatggaacattgataaatgttatacctcgcttttcatctcggggacaaattgagatgtctttaacgatagaggatggaacaggaaatagtcagtcaaattacaattataataatgaaaatacatctgtccttccagaagttggaaggaccaaaatcagtacaatagcgagagttccacaaggcaaaagtttgttgataggtggttatacgcatgaaacgaactctaacgaaataatatctattccttttctctcctctattcctgtaatcggtaatgtgttcaagtataaaacaagtaatatcagtaatattgtaagggtttttctgattcaaccccgtgagataaaggagtcaagttattataatactgcagaatataaaagcctcatatctgaaagagaaatacaaaagactacacaaataatcccatctgaaactactttgctggaagacgaaaaatcattggtttcatacttaaattactaactataaagtaggtgatgtatgcttgatgttaaaaatacaggagtttttagctctgcattcattgataagttgaatgcaatgacaaattcagatgatggagatgagactgctgatgcagagcttgattctggcttggctaatagcaagtatattgactcatctgatgagatggcttccgctctttcgtcatttataaacagaagagaccttgagaaactgaaaggaacaaatagtgatagtcaggaacgtattttagatggggaagaagatgaaattaatcacaagatttttgatttaaagagaacgttaaaagataaccttcctctagatcgggattttatagacagactaaagagatattttaaagatccaagtgatcaagtcttagcattaagggaacttttgaatgaaaaagatcttactgctgaacaagtcgaattattaactaaaattattaatgagataatatcaggtagtgaaaaaagtgttaatgctggaataaattcagctatacaggctaaattatttggcaacaaaatgaaacttgaaccacagcttttgcgtgcatgttatcgtggttttatcatggggaacatatcaacaacagatcagtatatagaatggcttggtaattttggttttaatcacagacatacaattgtgaattttgtagagcagtcactgattgtagacatggattctgagaaaccgagctgtaatgcttatgagtttggttttgtgttatctaaattaattgcaattaagatgattagaacttcagacgtaatttttatgaagaaactggaatcctcaagcttgctaaaagatggcagtttaagtgcagagcagctattgctaactttattatatatttttcaatatccaagtgaaagtgagcaaattcttacttctgttatagaagtatcacgagccagtcatgaggattctgtagtgtatcaaacatatctatcttctgttaatgaaagtcctcatgatatatttaaaagtgaaagtgaaagagaaattgcgatcaatattctacgagagcttgtcacaagtgcatacaagaaagagctttctagataacaggagataaaagtgatccagtcttttcttaagcaagtaagtactaagcctgaattaattatattagtgcttatggttatgatcattgctatgttgatcataccattacctacataccttgttgattttttaatagggcttaatattgtacttgctattctcgtttttatgggatcattttatattgaaagaatattgagtttttctacattcccttcggttttgttgataacgacattatttcgtctcgctttatcaataagtactagtcgactaattttagttgatgcggatgcgggtaaaattattactacatttggccagtttgtcattggtgatagtctggctgtaggttttgttattttttcaatagtaactgttgtgcagttcattgttataacaaaaggatctgaaagggttgcggaggtagctgctcgcttttctcttgacggtatgccggggaaacaaatgagcattgatgcggatttaaaggctggaattattgatgccgcaggagctaaagaaagacgcagtattctggaacgtgagagtcaattgtatggttcatttgatggagcaatgaagttcattaaaggtgatgcaattgctggcatcattattatttttgtcaatttaataggaggtatttctgttggtatgagtcaacatggaatgtccctatctggtgcattatctacttataccatactaactattggtgacggattggtctctcagattcctgctctgttaatttctataagtgcaggatttatcgttacaagagttaatggtgatagcgataatatgggacgtaacataatgtcccagatatttgggaatccttttgttctcattgttacatcagctcttgccttggcgatagggatgttgccaggttttccgttttttgtttttttcctgatagcagttactttgacggctttattctattataaaaaggtcgtagaaaaagaaaaaagtctgtctgagtctgattctagtggctatactggtacatttgatattgataatagccatgactcatctttggcaatgatagaaaatctggatgctattagttcagaaaccgttcctttaattttattatttgccgaaaataagataaatgcaaatgatatggaaggtcttattgaaaggataagaagtcagttctttattgattatggtgtcaggcttccaactattttatatagaacaagcaatgagcttaaggtcgatgatattgttttgttaataaatgaggtgcgtgccgatagttttaatatatattttgacaaagtgtgtattacagatgaaaatggagatatagatgctctaggtattcctgttgtttcaacttcatataatgaacgtgttatttcttgggtagacgtttcatatacagaaaatctaactaatattgatgctaaaattaaaagtgctcaagatgaattttatcaccagttgtcacaagctttattaaacaacataaatgagatttttggtatacaagaaacaaaaaatatgttagatcagtttgaaaatcggtatcctgatctattaaaggaagtcttccgacatgtgactatacagagaatttctgaggtattacaaagattgcttggagaaaatatttctgttcgcaatttaaaacttattatggagtctttggcgctttgggctcccagagaaaaagatgtcataacattagttgaacatgtccgtgcatcactttctaggtatatttgtagtaaaatagctgtttctggtgagattaaagttgtgatgctttccggatatattgaggatgcaataagaaaagggataaggcaaacctctggtggctctttcttgaatatggatatagaggtttcggatgaggtaatggaaactttagcacatgctttgagagaattgagaaatgcaaaaaaaaatttcgttcttttggtatcagtagatatacgtaggtttgttaaaagacttatagataacagatttaagagtatactcgttatatcgtatgctgagattgatgaagcatataccattaatgtattaaagactatttagtgaggtttaaatatgagtaacattaatttagttcaattagttagagatagtcttttcacgattggttgtcctccatcaattattacagatttagacagccattcagcgattaccatttctttggatagtatgcctgctattaatatagctcttgtcaatgagcaggttatgttatgggctaattttgatgcaccaagtgatgtaaaacttcagtcatctgcttataacatattaaatttaatgttaatgaactttagttatagcattaatgagctggtagagcttcatcgttctgatgagtacttacagttaagagttgttattaaagatgactacgttcacgatgggattgtttttgcagagattctgcatgagttttatcagaggatggaaatcttaaatggggtcttataatcaatgagctatacaaaattgctcactcaattatcttttcctaatagaatctcggggccaatcttggaaacaagtcttagcgatgtttcgattggtgagatttgtaacattcaggctggaattgaaagtaatgaaattgttgcaagagctcaggttgtaggatttcatgatgaaaaaacaatattaagcttgattggaaattctcgtggactttcacggcaaacgttgattaagcccactgcccagtttcttcatacgcaagtcggccgtggattattgggagcagtagtcaatcctttaggggaggttactgataagtttgctgttacagataacagtgaaattctttatcgacctgtagataatgctcctccgctatatagtgaaagggctgcaattgagaagccttttttaacaggtattaaggttattgattctttactcacgtgtggtgaaggacagcgaatggggatttttgcgtcagctggttgtggcaaaacttttctcatgaatatgctcattgaacatagtggtgctgatatatatgttattgggttaattggtgagcgaggtcgagaggttactgaaacggttgattatttgaaaaactctgagaaaaaaagcaggtgtgttttagtatatgcaacttcggattactcttcggttgatcgttgtaatgctgcatatatagccactgctatagccgaattttttaggactgaaggacataaagtagcgctttttattgattcattaacaaggtatgccagagcattacgtgatgtggccttagccgctggagaatcacctgccagaagaggctatccggtttcggtttttgatagcttacccagacttcttgaaaggccaggaaagttaaaggcaggtggctctattactgcattttacactgttcttttggaggatgatgattttgctgatccattagctgaagaggtaagatccattttagatggacatatatatttgagcagaaatctagcccaaaaaggacaatttcctgcaattgattccttaaaaagtataagcagggtatttacacaggttgttgatgaaaaacatcgtattatggccgctgcatttcgggagctactttctgaaatagaagagctaaggacaattattgactttggtgaatacaaaccgggggagaatgcctctcaggataaaatctacaacaaaatatctgttgttgagagttttctgaagcaagattatcgactcggttttacttatgagcagacaatggagcttattggtgaaacaattagataaggttttaaaaatcaaagataaatatcaacggtctgttaaattaattgaagcacacatattaacactaaaaaaaaaactctttgtatcgagatgtggaggcattagataaaaggattatatacttcctgcaactggagaatgacttagaacctgtaggcgctcagagtgttagtcagcttttcaatacaagacgtaagatcgctatagtaaaaaagcatattatccaatatcagtctgaacgaattcttctaaagggaagaattgaagaaattcaaaaagatattgatgaggcaaatgcttctaaaagaaaactgcttcataaagagagtaaaatatgtaaacggattggcttgattaaaagaaataactttgccaaacagttgatacttgatgagttatcacaagaggatatgaagtatggcattagataatataaacctaaattttagtagtgacaaacagatagaaaaatgtgagaaactatcttcaatagataatattgatagtctcgttttgaagaagaagaggaaggttgaaattcctgagtactctttaattgcatctaactattttactattgataagcactttgaacataagcatgataaaggagaaatttatagtggcattaaaaatgcgttcgaacttagaaacgaacgagcgacatattctgatattccagaatcaatggccattaaagaaaatattttgataccagatcaagatatcaaagcaagggaaaaaataaatatcggcgatatgagggggatcttttcatataataagagtggaaatgcagacaagaacttcgaaagaagtcatacttcttctgtaaaccctgataatctgctagaatctgataatagaaatggtcaaattggtttaaaaaatcatagcttgtctattgataagaatattgctgacatcatttctttactaaatggaagtgttgctaaatcatttgagctgcctgtaatgaataaaaatactgcagacataaccccatccatgtcattgcaagaaaaatcaatagttgaaaatgataaaaatgtttttcaaaaaaatagtgaaatgacttaccactttaaacagtggggggctggacattctgttagtatttcagtggagtctggttcttttgttctaaaaccgtcagatcaatttgtaggaaataaacttgacttaattttgaaacaagatgctgagggtaattacagatttgatagcagtcaacataataaggggaataaaaataatagtacaggatataatgaacagagtgaagaagaatgctaagaattaaacattttgacgctaacgaaaaactacagattttatatgcaaagcaactctgcgagcgtttttcgattcagacattcaaaaataaatttacaggcagtgaaagtttagtcactcttacttccgtgtgtggggattgggtaattcgtattgatacattatcttttttgaaaaaaaaatacgaggtattttcaggattttctacacaagaatctttactgcatttatcaaaatgtgtctttatagagtcgtcatctgtattttcgattccagaactgtctgataagattactttccggatcacgaatgaaatccagtatgcaactactgggagtcatttatgctgtttttcctcttctttaggtattatttattttgacaagatgccggtattacgtaatcaagtttctcttgactcattgcatcatcttttagagttttgcttaggttcatctaatgtaaggctggctactttaaaaagaattcgcactggtgatataatcatagttcagaaactttataatttattattgtgtaatcaagttattattggggattatattgtgaatgataataatgaggcaaaaattaatctgtcagaaagtaatggtgagtcagaacacacagaagtttctttggcattattcaattatgatgatatcaatgtaaaagtggactttattcttttagaaaaaaatatgacaatcaatgaactaaaaatgtatgtagaaaacgaattattcaagtttcccgatgacatagttaaacatgtaaatattaaagtaaatggttctttggttgggcatggggaacttgtttctattgaggatggttatggtatcgagattagttcttggatggtaaaggagtaatctcatgctgagtgacatgtccctcatcgctacactttctttttttacgctattaccatttttagttgctgctggtacctgttatataaagttttctattgtttttgtaatggtacgaaatgcacttggtttgcagcaggttccatcaaatatgactctcaatggtattgcattgattatggctttgttcgtgatgaaacccattattgaagctggttatgaaaattatctaaatggccctcaaaaatttgatacaatatctgatattgtccgtttttctgattctggcttgatggaatataagcaatacttgaaaaagcatacagatcttgagttagcacgttttttccaaaggtctgaagaagaaaatgctgatttaaagagtgcagagaataatgattattcattgt

SEQ ID NO: 6 - DNA, 930 bp, Artificial Sequence
  Synthetic Construct
atggtggatttgtgcaacgacttgttaagtataaaggaaggccaaaagaaagagtttacactccattctggtaataaagtttcctttatcaaagccaagattcctcataaaaggatccaagatttaaccttcgtcaaccaaaaaacgaatgtacgcgatcaagaatccctaacagaagaatcattagccgatatcataaaaactataaagctacaacaattcttccctgtaataggaagggagattgatggtagaattgaaattctggatggcactcgtagaagagcatctgcaatatatgcaggagcagatcttgaagttctatattcaaaagaatatatatctactcttgatgccagaaaactagcaaacgatatacaaacagcaaaagagcatagcatccgagaacttggtattggtcttaattttctgaaagtatcagggatgtcctataaagacatagccaaaaaagagaatctgtctcgcgcgaaagtcactcgtgcctttcaggcagcaagcgttccacaggaaataatatctctatttccaatcgcgtcagaacttaactttaatgactacaagatattattcaattattataaaggacttgaaaaggctaatgaatctcttagttctacactaccaatattaaaggaagaaataaaagaccttgatacaaatttgcccccggacatatataaaaaagaaattttaaacatcataaagaaaagcaaaaacagaaagcaaaacccttcgctaaaagttgactcgttatttatttctaaagacaaacggacttacataaaaagaaaagagaataaaacaaacagaactctaatatttacattatctaaaataaacaaaacagttcagagagaaatagatgaagctattcgggatataatttctcgccatctatcgtcttcataa

SEQ ID NO: 7 - DNA, 7104 bp, Artificial Sequence
  Synthetic Construct
  misc_feature (5650)..(5650): n is a, g, c, or t
gatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattccacatgtggaattccacatgtggaattgtgagcggataacaatttgtggaatcccgggagagctcaggttacccgcatgcaagatctatctagaagggccccactagtgacgcgtactcgagggtcgacggtatcgataagcttgatatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaargaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagwttgctcagctctcccgtggaggtaataattgacgatatgatcatttattctgcctcccagagcctgataaaaacggttagcgcttcgttaatacagatgtaggtgttccacagggtagccagcagcatcctgcgatgcagatcatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctatnggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagag

SEQ ID NO: 8 - DNA, 4037 bp, Artificial Sequence
  Synthetic Construct
agaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcccctggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagtagtcttccgacagtcgaagactaagcggacacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaataaatgcctcggtaccaaattccagaaaagaggcctcccgaaaggggggccttttttcgttttggtcccgtctctgttccttatcatctggcgaatcggacccacaagagcactggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataataacgtaaaaacccgcttcggcgggtttttttatgcaagacgctggctctgacatcgagtagttcagtagcggaaaacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctca

SEQ ID NO: 9 - DNA, 3332 bp, Artificial Sequence
  Synthetic Construct
tgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattacgccccgccctgccactcatcacagtactgttgtaattcattaagcatgcggccgacatggaagccatcacaaacggcatgatgaacctggatcgccagcggcatcagcaccttgtcgccttgcgtataatatttgcccatcgtgaaaacgggggcgaagaagttgtccatattggccacgtttaaatcaaaactggtgaaactcacccagggattggctgagacaaaaaacatattctcaataaaccctttagggaaataggccaggttttcaccgtaacacgccacatcttgcgaatatatgtggagaaactgccggaaatcgtcgtggtattcactccagagggacgaaaacgtttcagtttgctcatggaaaacggtgtaacaagggtgaacactatcccagatcaccagctcaccgtctttcatggccatacgaaactccgggtgagcgttcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatcccgtgatttttttctccattttagcttccttagctcctgaaaatctcgataactcaaaaaatacgcccggtagtgatcttatttcattatggtgaaagttggaacctcttacgtgcccgatcaaccagtgctcttgtgggtccgattcgccagatgataaggaacagagacgggaccaaaacgaaaaaaggcccccctttcgggaggcctcttttctggaatttggtaccgaggcatttattaccaatgcttaatcagggaggcacctatctcagcgatctggcgattacgttcgtccatagttgcctggctccccgtcgtgtagataactacgatacgggagggcttaccatctggccccagggctgcaataataccgcgggacccacgctcaccggctccagatttatcagcaataaaccagccagccggaagggccgagcgcagaagtggtcctgcaactttatccgcctccatccagtctattaattgttgccgggaagcgagagtaagaagttcgccagttaagagtttgcgcaacgttgttgccatagctacaggcatcgtggtgtcacgctcgtcgtttggtatggcttcattcagctccggttcccaacgatcaaggcgagttacatgatcacccatgttgtgcaaaaaagcggtaagctccttcggtcctccgatcgttgtcagaagtaagttggccgccgtgttatcactcatggttatggcagcgctgcataattcgcgtactgtcatgccgtccgtaagatgcttttctgtgactggtgagtactcaaccaagtcattctgagaatagtgtatgcggcgaccgagttgctcttgcccggcgtcaatacgggataataccgcgccacagagcagaactttaaaagtgctcatcattgggaaacgttcttcggggcgaaaactctcaagaatcttaccgctgttgaggtccagttcgatgtaacccacacgtgcacccaactgatcttcggcatcttttactttcaccagcgtttctgggtgtccgcttagtcttcgactgtcggaagactacttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagt

SEQ ID NO: 10 - DNA, 8217 bp, Artificial Sequence
  Synthetic Construct
  misc_feature (8150)..(8150): n is a, g, c, or t
tggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattccacatgtggaattccacatgtggaattgtgagcggataacaatttgtggaatcccccttgagcaaggcagaagtctcagaaagaagccgagttactgttcgttcttttgctgagctttttgttctttgtacttttctaataggttttccggcggtggaggaacctgcagaaagaagccatcattttgtaatgaagccttcactttttcgatatctacttgtgccaatttacgaccatcaagtttaatcgtcatgacaggaataggcttaccgaacatctgcattaaggtgtcaggaacctgtgaaaaatcatcctttttcgggatatagagataagtaccttcttttttcgagcttttataaatagaacaaagcatgacacgccttttatgctgatttacccgctttgaatacggattgagttgaaaacgtaaaattctcgcaaacttaattgcaaatttgagattaattcgcctatttgcctgctagagaacttgctgcgtcaaaggatgaaatataacatgacagacctagtttaaggcaacgctctttcaacagaggcaagaagtccacgggctttattctcaacgcaatatgcagcgcacaacaatgtacgtgtcgaaaaaaggttgaccatttggtcaacctttttagtttgcgcttatttcaacgccttattcattttttcgcccaataactctagacgccaaccttgcataacatcaggcaagcgagcaggatcacgatccttcttccacacccaactgatgagctggttaatctgctttttcgatgctaaaaactcggtcgctaaccctgatttttgcgcagcacttttcacgacatctttcatgtttttgaaaacttgcttatagccagggtaatccatcaggcgatcaattttagctgggtattcttctggtggcgtttgcttagcctttttcacgatggccgcaattttagtaccgtgacgattgaccgcgcggctatccatcccttccgcttccatctttttaaagcttgttaaacctaaacgagcaaccgttagcaaatcaccctctttgaagatgaagtttagtgctaagtcgcgcttgattgcttcacgataacgccaagtcgctagcggctttaacacagctaactctgacggtttaagttgccacgctcctgggagagctcaggttacccgcatgcaagatctatctagaagggccccactagtgacgcgtactcgagggtcgacggtatcgataagcttgatatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaargaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagwttgctcagctctcccgtggaggtaataattgacgatatgatcatttattctgcctcccagagcctgataaaaacggttagcgcttcgttaatacagatgtaggtgttccacagggtagccagcagcatcctgcgatgcagatcatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctatnggcattttcttttatttcttatcaacataaaggtgaatcccatatgaactatataaaagcaggcaaa

SEQ ID NO: 11 - DNA, 9299 bp, Artificial Sequence
  Synthetic Construct
  misc_feature (7845)..(7845): n is a, g, c, or t
gatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattccacatgtggaattccacatgtggaattgtgagcggataacaatttgtggaatcccccttgagcaaggcagaagtctcagaaagaagccgagttactgttcgttcttttgctgagctttttgttctttgtacttttctaataggttttccggcggtggaggaacctgcagaaagaagccatcattttgtaatgaagccttcactttttcgatatctacttgtgccaatttacgaccatcaagtttaatcgtcatgacaggaataggcttaccgaacatctgcattaaggtgtcaggaacctgtgaaaaatcatcctttttcgggatatagagataagtaccttcttttttcgagcttttataaatagaacaaagcatgacacgccttttatgctgatttacccgctttgaatacggattgagttgaaaacgtaaaattctcgcaaacttaattgcaaatttgagattaattcgcctatttgcctgctagagaacttgctgcgtcaaaggatgaaatataacatgacagacctagtttaaggcaacgctctttcaacagaggcaagaagtccacggttccttatcatctggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaaggctttattctcaacgcaatatgcagcgcacaacaatgtacgtgtcgaaaaaaggttgaccatttggtcaacctttttagtttgcgcttatttcaacgccttattcattttttcgcccaataactctagacgccaaccttgcataacatcaggcaagcgagcaggatcacgatccttcttccacacccaactgatgagctggttaatctgctttttcgatgctaaaaactcggtcgctaaccctgatttttgcgcagcacttttcacgacatctttcatgtttttgaaaacttgcttatagccagggtaatccatcaggcgatcaattttagctgggtattcttctggtggcgtttgcttagcctttttcacgatggccgcaattttagtaccgtgacgattgaccgcgcggctatccatcccttccgcttccatctttttaaagcttgttaaacctaaacgagcaaccgttagcaaatcaccctctttgaagatgaagtttagtgctaagtcgcgcttgattgcttcacgataacgccaagtcgctagcggctttaacacagctaactctgacggtttaagttgccacgctcctgggagagctcaggttacccgcatgcaagatctatctagaagggccccactagtgacgcgtactcgagggtcgacggtatcgataagcttgatatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaargaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagwttgctcagctctcccgtggaggtaataattgacgatatgatcatttattctgcctcccagagcctgataaaaacggttagcgcttcgttaatacagatgtaggtgttccacagggtagccagcagcatcctgcgatgcagatcatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctatnggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagag

SEQ ID NO: 12 - DNA, 3817 bp, Artificial Sequence
  Synthetic Construct
tatggacgaacgtaatcgccagatcgctgagataggtgcctccctgattaagcattggtaataataacgtaaaaacccgcttcggcgggtttttttatgcaagacgctggctctgacatcgagtagttcagtagcggaagcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagactagaaggaggaaaaaaatgatttgcagggtattagatataaacatgaaaattccctcattacagtccaacttcaacttttccgccccggcaggatactctgctcccattgctcctaatcgtgctgaaaatgcctatgcggattacgttttggatataggtaagcgaataccactttccgcagcagatttaagcaacgtatacgaaagtgtaatacgcgccgtccatgacagccgtagcaggcttatcgatcagcatacagtcgatatgatcggcaacactgtacttgatgctttgagccgatcacagacatttcgtgatgccgtaagctatggcattcataatgagaaggtacacattggttgcattaaatacagaaacgaatacgagcttaacgaagaatcttctgtcaaaattgatgatattcaatcactaacctgtaacgaattatatgaatatgatgtcgggcaagagccaattttccccatttgcgaagcaggagaaaacgataacgaagagccttatgtcagttttagtgttgcgccagatactgactcttatgagatgccatcgtggcaggaaggactgattcacgagattattcatcatgttactggatctagcgatccatctggagatagtaatatagagttaggacccaccgagattctcgcacgtcgtgtcgctcaagaactgggatggagtgttcccgacttcaaaggatatgcagagccagaacgtgaagctcatcttaggctacgtaacctgaatgcccttcgacaggctgccatgaggcatgaagagaatgagagggctttcttcgaaaggctgggtacgatcagtgaccgatatgaggcgagtcctgatttcacagagtattccgctgtgtctaacataggatacggatttatccagcaacatgattttcctggattggctatcaacgataatttacaggatgcaaatcagatccaactgtatcatggcgccccttatatttttacatttggggatgtggacaaacacaatcagcaagggggaagtgactacaaagacgatgatgataaataaatgcctcggtaccaaattccagaaaagaggcctcccgaaaggggggccttttttcgttttggtcccgtctctgttccttatcatctggcgaatcggacccacaagagcactgctaaatacattcaaatatctatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaac

SEQ ID NO: 13 - DNA, 3517 bp, Artificial Sequence
  Synthetic Construct
tatggacgaacgtaatcgccagatcgctgagataggtgcctccctgattaagcattggtaataataacgtaaaaacccgcttcggcgggtttttttatgcaagacgctggctctgacatcgagtagttcagtagcggaagcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagactagaaggaggaaaaaaatgcccatcaaaaaaccgtgtctgaaactcaatttagattccctgaatgttgtgcgttccgaaattccgcagatgctttcggcgaatgaacgcctgaaaaataacttcaacattttgtacaaccaaattcggcagtacccagcgtactatttcaaagtagcctcgaacgtgccgacctactccgatatctgtcaatctttttcggtcatgtaccaaggcttccagattgtgaaccacagtggcgacgtgtttattcacgcatgccgtgaaaatcctcagagcaagggcgattttgttggggacaagtttcatatttccattgcccgtgaacaggtccctctggcattccagattttgagtggtttgttattttcagaggacagtccgatcgataaatggaagatcactgacatgaatcgcgtttctcagcaaagccgtgtgggtatcggagctcagttcacgctatatgtaaaatcagatcaggaatgctcgcaatatagtgccctgctgcttcataaaatacgacagtttataatgtgcctcgagtctaacctgttacggagcaaaatcgctcccggtgagtatccggcgtcagacgttagaccggaagattggaaatatgtcagctatcgtaacgaactgcgcagcgatcgcgatggcagcgaacgccaggaacaaatgctgcgcgaggaaccattttatcgtctgatgatcgaggggggaagtgactacaaagacgatgatgataaataaatgcctcggtaccaaattccagaaaagaggcctcccgaaaggggggccttttttcgttttggtcccgtctctgttccttatcatctggcgaatcggacccacaagagcactgctaaatacattcaaatatctatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaac

SEQ ID NO: 14 - DNA, 3578 bp, Artificial Sequence
  Synthetic Construct
caaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagcagtgctcttgtgggtccgattcgccagatgataaggaacagagacgggaccaaaacgaaaaaaggcccccctttcgggaggcctcttttctggaatttggtaccgaggcattcaattagcgatggcgacttgctcatcaccggaacggaccttacttggcgtagtgccaaagcgacgtcgataactttgagtaaagtaagattggctggaaaatcccgcttccattgccacgtcgacaatgctcattttgctcgtcagcaaaagcttgtgtgcgtgcaataagcgacgctcactgatccaagctcttggagagatgccatagtgctcattaaaaagctctttaaaggttgtgagactggcaccaaattcttgagcataatccgtcagcttccactctttcaaatagtgctgctccataaagcggcgtaagcggtctgaggttcggttagtttgctgcgatagcaaggcatagaggacttcgcccccttgactgtgcagcaatagaatcagcatttcttcgtacttcaaacgcatgatggcgtctgggtacgactgctcctccagcgttttcatgccggaaagcacttgctcaatgagaatgttcaaaccaaaagaaaacaaacccgagttgagtttgtccacttttggcaacgtcatgagaagatcgctgtgtttttgcagtaattggcttactagctcattgtcgaattcaatgatagatgagtgataattcccaccctgtggtgagtactctaacaagtagtcaccttgagagagaaataccccactcccggcaacgacatctatggtgcaatcgccagtttgaactcgtaattgaccattgtgaacgacaacaatatgagattggtctgagtgaaatacttcgtcatgtttttcgtagtgagaaaaactgcgaatttttcgcaagctagagccgaccgtttctgtgtttagttggcctgacacatccattttctacccttccttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagat

SEQ ID NO: 15 - DNA, 3520 bp, Artificial Sequence
  Synthetic Construct
tggcgaatcggacccacaagagcactgctaaatacattcaaatatctatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaataataacgtaaaaacccgcttcggcgggtttttttatgcaagacgctggctctgacatcgagtagttcagtagcggaagcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatctggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagactagaaggaggaaaaaaatgacaaaaataactttatctccccagaattttagaatccaaaaacaggaaaccacactactaaaagaaaaatcaaccgagaaaaattctttagcaaaaagtattctcgcagtaaaaaatcacttcatcgaattaaggtcaaaattatcggaacgttttatttcgcataagaacactgaatcttctgcaacacactttcaccgaggaagcgcatctgagggccgggcagtgttgacaaataaagtcgttaaagattttatgcttcaaacgctcaatgatatagatattagaggtagtgcgagtaaagaccccgcatacgccagccagacccgtgaagctatactatcggcagtttacagcaagaataaagatcagtgttgtaatttgctcatcagcaaagggatcaacatagcgccttttcttcaggaaattggcgaagcagcgaaaaatgtaggtctgcccggaacaaccaaaaatgacgtttttacgccaagcggcgcaggggccaatccttttataactccgttgatttcatcagcaaacagtaagtatccacgtatgtttatcaatcaacatcagcaggcatcctttaaaatctatgcggagaagatcattatgacagaagttgcaccactgtttaatgagtgtgctatgccgactccacagcaattccaactgatactagaaaacattgctaataaatatatacaatacactcccgggggaagtgactacaaagacgatgatgataaataaatgcctcggtaccaaattccagaaaagaggcctcccgaaaggggggccttttttcgttttggtcccgtctctgttccttatcatc

SEQ ID NO: 16 - DNA, 2759 bp, Artificial Sequence
  Synthetic Construct
ggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctaaagccagttaccttcggaaaaagagttggtagctcttgatccggcaaacaaaccaccgctggtagcggtggtttttttgtttgcaagtagcagattacgcgcagaaaaaaaggatctcaagaagatcttttgatcttttctacgggtggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagcgagaccgctgggagttcgtagacggaaacaaacgcagaatccaagcggtctccatgcctcggtaccaaattccagaaaagaggcctcccgaaaggggggccttttttcgttttggtcccgtctctgttccttatcatct

SEQ ID NO: 17 - DNA, 3702 bp, Artificial Sequence
  Synthetic Construct
ggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctaaagccagttaccttcggaaaaagagttggtagctcttgatccggcaaacaaaccaccgctggtagcggtggtttttttgtttgcaagtagcagattacgcgcagaaaaaaaggatctcaagaagatcttttgatcttttctacgggtggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagcaattactgaactaaggaggtagtaatgaagatccctgaagctgtgaatcatattaatgtacaaaataatattgatctggtggatggcaaaattaatccaaataaagacactaaagctctgcaaaaaaatattagttgcgtaacgaattcatcatcatctggaatcagtgagaagggcggtggtggttcgggcggtggtggtagtcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaaatgcctcggtaccaaattccagaaaagaggcctcccgaaaggggggccttttttcgttttggtcccgtctctgttccttatcatct

SEQ ID NO: 18 - DNA, 3864 bp, Artificial Sequence
  Synthetic Construct
ggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctaaagccagttaccttcggaaaaagagttggtagctcttgatccggcaaacaaaccaccgctggtagcggtggtttttttgtttgcaagtagcagattacgcgcagaaaaaaaggatctcaagaagatcttttgatcttttctacgggtggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagcaattactgaactaaggaggtagtaatgacaaaaattactctgtcgccacagaatttccggatccaaaaacaggaaaccacgctgctgaaagagaaatcaaccgagaagaactctttagctaaaagtattctcgcagtgaagaatcatttcatcgaattacgctcaaaactgtcggaacgttttatttcgcataagaacaccgaaagttccgcgactcactttcaccgcgggagcgcgtctgagggccgcgcagtgttgacgaataaagtcgttaaagattttatgcttcaaacgctgaacgatatagatattcgtggtagtgcgtccaaagacccgggcggtggtggttcgggcggtggtggtagtcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaaatgcctcggtaccaaattccagaaaagaggcctcccgaaaggggggccttttttcgttttggtcccgtctctgttccttatcatct

SEQ ID NO: 19 - DNA, 3714 bp, Artificial Sequence
  Synthetic Construct
ggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctaaagccagttaccttcggaaaaagagttggtagctcttgatccggcaaacaaaccaccgctggtagcggtggtttttttgtttgcaagtagcagattacgcgcagaaaaaaaggatctcaagaagatcttttgatcttttctacgggtggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagcaattactgaactaaggaggtagtaatgcatatccaatcgcttcaacagtccccgtcttttgctgttgaattgcaccaggcagccagtgggcgtttaggccagattgaagcacggcaagtggccaccccgagcgaagcgcagcagctggcgcagcgccaggatgcgccaaaaggtgagggcctgctgggcggtggtggttcgggcggtggtggtagtcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaaatgcctcggtaccaaattccagaaaagaggcctcccgaaaggggggccttttttcgttttggtcccgtctctgttccttatcatct

SEQ ID NO: 20 - DNA, 3702 bp, Artificial Sequence
  Synthetic Construct
ggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctaaagccagttaccttcggaaaaagagttggtagctcttgatccggcaaacaaaccaccgctggtagcggtggtttttttgtttgcaagtagcagattacgcgcagaaaaaaaggatctcaagaagatcttttgatcttttctacgggtggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaagcaattactgaactaaggaggtagtaatgaaaatctcaagctttatttcgacttcactgccactgccgacgtctgtgtccggttcgagcagcgttggcgaaatgagtgggcgctccgtctctcagcaaaccagtgatcagtatgccaacaatttagcgggccgtaccgagagcccgggcggtggtggttcgggcggtggtggtagtcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaaatgcctcggtaccaaattccagaaaagaggcctcccgaaaggggggccttttttcgttttggtcccgtctctgttccttatcatct

SEQ ID NO: 21 - DNA, 59 bp, Artificial Sequence
  Synthetic Construct
ggatgagagggatgaaaaactcaaggcagagataactctgattacacgtcttgagcgat

SEQ ID NO: 22 - DNA, 59 bp, Artificial Sequence
  Synthetic Construct
ataaatgggagggtgacttgcctcaatataatccagactactgacatgggaattagcca

SEQ ID NO: 23 - DNA, 57 bp, Artificial Sequence
  Synthetic Construct
ctttgccataaaattctgctgggatttgatcactcagcattacacgtcttgagcgat

SEQ ID NO: 24 - DNA, 56 bp, Artificial Sequence
  Synthetic Construct
ctggcgatgattaatagctaattgagtaaggccaggactgacatgggaattagcca

SEQ ID NO: 25 - DNA, 59 bp, Artificial Sequence
  Synthetic Construct
acaagcgtttgaggaagcctttcttctcttcttcaatgaaattacacgtcttgagcgat

SEQ ID NO: 26 - DNA, 59 bp, Artificial Sequence
  Synthetic Construct
caaggaatttctatggcacgcattattgttgttacttcggctgacatgggaattagcca

SEQ ID NO: 27 - DNA, 59 bp, Artificial Sequence
  Synthetic Construct
ttgagtaaggccaggatgtcaaacacgccaatcgagcttttgtgtaggctggagctgct

SEQ ID NO: 28 - DNA, 60 bp, Artificial Sequence
  Synthetic Construct
ctttttgctcaagctggacggtaaccatttccggatcgatcggctgacatgggaattagc

SEQ ID NO: 29 - DNA, 539 bp, Artificial Sequence
  Synthetic Construct
atgctaaagtatgaggagagaaaattgaataatttaacgttgtcttcgttttcaaaagttggtgtgtcgaatgatgcccgactttatattgctaaggaaaatactgataaggcatatgttgcgcctgaaaaattttcgtcaaaagtattaacctggcttggaaaaatgccgttatttaaaaacactgaagtggtgcaaaaacatacggaaaatatcagagtacaggaccaaaagattttacagacatttctccatgcactaacggaaaaatatggggaaacagcggttaatgacgcactgttaatgtcccgtataaatatgaacaaaccccttacccaacgtttagcagtgcagatcacggagtgtgtaaaagctgctgacgaagggtttataaaccttattaagagcaaggataatgttggtgtcaggaatgccgctttagtcataaaaggcggcgatacaaaagtggcagaaaaaaataacgatgttggagcagaaagtaagcaacctttactcgatatcgcgctaaagggacttaa

SEQ ID NO: 30 - DNA, 150 bp, Artificial Sequence
  Synthetic Construct
atgccagtcactttaagcttcggtaatcatcaaaattatacgcttaatgaaagtcggcttgctcatctgttaagcgcagataaagaaaaagcaatccatatggggggttgggataaagtccaggatcattttagagcggaaaaaaaggac

SEQ ID NO: 31 - DNA, 540 bp, Artificial Sequence
  Synthetic Construct
atggttacaagtgtaaggactcagccccccgtcataatgccaggtatgcagaccgagatcaaaacgcaggccacgaatcttgcggcgaatctttccgcagtcagagaaagtgccacagcgacgctgtcaggggaaattaaaggcccgcaactggaagattttcccgcgctgatcaaacaggcgagtctggatgcattgtttaaatgcgggaaagacgctgaggcgttaaaagaagtttttaccaattcaaataatgtcgccggtaagaaagcgataatggagtttgccgggctctttcgttcagcgctcaacgccaccagtgattctcctgaggcgaagacgctactgatgaaggtgggggcagagtataccgcgcaaatcataaaagatggcctgaaagaaaagtcagcttttgggccatggctgccagaaacaaagaaagcggaagcgaagctggaaaacctggaaaagcagctgttagatattatcaaaaataacactggcggtgaattaagtaaattatcgacgaatcttgttatg

SEQ ID NO: 32 - DNA, 600 bp, Artificial Sequence
  Synthetic Construct
atgtttaatattactaatatacaatctacggcaaggcatcaaagtattagcaatgaggcctcaacagaggtgcctttaaaagaagagatatggaataaaataagtgcctttttctcttcagaacatcaggttgaagcacaaaactgcatcgcttatctttgtcatccacctgaaaccgcctcgccagaagagatcaaaagcaagtttgaatgtttaaggatgttagctttcccggcgtatgcggataatattcagtatagtagaggaggggcagaccaatactgtattttgagtgaaaatagtcaggaaattctgtctatagtttttaatacagagggctataccgttgagggagggggaaagtcagtcacctatacccgtgtgacagaaagcgagcaggcgagtagcgcttccggctccaaagatgctgtgaattatgagttaatctggtctgagtgggtaaaagaggcgccagcgaaagaggcagcaaatcgtgaagaagccgtacaacggatgcgtgactgcctgaaaaataataagacggaacttcgtctgaaaatattaggacttaccactatacctgcctatattcctgagcag

SEQ ID NO: 33 - DNA, 300 bp, Artificial Sequence
  Synthetic Construct
atgaagatatcatcaggcgcaattaatttttctactattcctaaccaggttaaaaaattaattacctctattcgtgaacatacgaaaaacgggctcacctcaaaaataaccagtgttaaaaacacgcatacatctttaaatgaaaaatttaaaacaggaaaggactcaccgattgagttcgcgttaccacaaaaaataaaagacttctttcagccgaaagataaaaacaccttaaacaaaacattgattactgttaaaaatattaaagatacaaataatgcaggcaagaaaaatatttca

SEQ ID NO: 34 - DNA, 540 bp, Artificial Sequence
  Synthetic Construct
atgcaaatacagagcttctatcactcagcttcactaaaaacccaggaggcttttaaaagcctacaaaaaaccttatacaacggaatgcagattctctcaggccagggcaaagcgccggctaaagcgcccgacgctcgcccggaaattattgtcctgcgagaacccggcgcgacatgggggaattatctacagcatcagaaggcgtctaaccactcgctgcataacctctataacttacagcgcgatcttcttaccgtcgcggcaaccgttctgggtaaacaagacccggttctaacgtcaatggcaaaccaaatggagttagccaaagttaaagcggaccggccagcaacaaaacaagaagaagccgcggcaaaagcattgaagaaaaatcttatcgaacttattgcagcacgcactcagcagcaggatggcttacctgcaaaagaagctcatcgctttgcggcagtagcgtttagagatgctcaggtcaagcagcttaataaccagccctggcaaaccataaaaaatacactcacgcat

SEQ ID NO: 35 - DNA, 540 bp, Artificial Sequence
  Synthetic Construct
atgttaattagtaatgtgggaataaatcccgccgcttatttaaataatcattctgttgagaatagttcacagacagcttcgcaatccgttagcgctaaagatattctgaatagtattggtattagcagcagtaaagtcagtgacctggggttgagtcctacactgagcgcgcctgcgccaggggtattaacgcaaacccccggaacgatcacgtcctttttaaaagccagtattcaaaataccgacatgaatcaggatttgaatgctctggcaaataatgtcacgactaaagcgaatgaggttgtgcaaacccagttacgcgagcagcaggcagaagtcggaaagttttttgatattagcggaatgtcttccagtgccgttgcgctgttggctgccgcgaatacgttaatgctgacgttgaaccaggctgatagcaaactgtctggtaagttgtcattagtcagttttgatgcagctaaaacgacggcaagctccatgatgcgcgaagggatgaatgcgttgtccggtagtatttcccag

SEQ ID NO: 36 - DNA, 300 bp, Artificial Sequence
  Synthetic Construct
atgatattttcggtgcaggagctatcatgtggagggaaaagtatgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctata

SEQ ID NO: 37 - DNA, 867 bp, Artificial Sequence
  Synthetic Construct
atgaatttagatggtgttagaccatactgtagaatagtcaataaaaagaatgaaagcatatcagatattgcatttgcacatataataaaaagggtaaaaaattcatcatgtactcacccaaaagcagcattggtttttttaggagagaaaggtttttgtgatagcaatgatgttctatctattatgggacaacaaataccaagagtatttaagaacaagatgttatatgattatgtttttaaaaatgaaaaaagtaaaaatgattttctaaaaatggctgaatcatggctaccacagagtgaaccaatagtaataaataatgatgatgacgcattgaatgctgctgcttatttttctgtaaaaaaagcgaaaataaaaacagtaaacgatactgattttaaagagtataataaggtttatattcttgggcacggtagtcctggttctcatcaattaggccttggttcggaacttattgatgtacaaacaatcatttcaagaatgaaagactgtggtattctaaatgtgaaagatatccgttttacttcatgcggctccgctgataaagtggctcctaaaaattttaacaatgcccctgctgaaagtctttcttgtatccttaactctctgcctttttttaaggaaaaagaatctttgctagagcagataaaaaaacaccttgaaaacgatgagtcattgagtgatggtctaaaaatatccggctatcatggatatggagttcactatggtcaagagctttttccctactcacattatcgttcaacttcaattcctgctgatccggagcatacagtaaaaagaagctctcagaaaaagacttttattattaataaagaactggattag

SEQ ID NO: 38 - DNA, 360 bp, Artificial Sequence
  Synthetic Construct
gtgactaacataacactatccacccagcactacagaatccatagaagtgacgttgaaccagtaaaagaaaaaacaacggagaaggacatttttgcaaaaagtattactgccgttagaaatagctttatcagcctgtcgacgagtctgtcagatcgttttagcctgcatcaacaaacagacataccgactacccattttcatcgtgggaacgcttctgagggtagggcggtattaaccagtaaaactgttaaagattttatgctgcaaaagctcaatagtctggatatcaaaggtaatgcgagtaaagatccggcctatgctcgtcagacatgcgaagccatattatcagccgtgtacagt

SEQ ID NO: 39 - DNA, 180 bp, Artificial Sequence
  Synthetic Construct
atgttaccgataaataataacttttcattgccccaaaattctttttataacactatttccggtacatatgctgattacttttcagcatgggataaatgggaaaaacaagcgctccccggtgaagagcgtgatgaggctgtctcccgacttggtggaggtggctccggcggcggaggttcc

SEQ ID NO: 40 - DNA, 180 bp, Artificial Sequence
  Synthetic Construct
atgaaaatacctgaagcagtaaatcatattaatgttcagaacaatattgatcttgttgatggaaaaataaatccgaacaaagatacaaaagcattacaaaaaaacatatcatgcgtaacaaactcatcctcttctggcataagtgaaaaaggtggaggtggctccggcggcggaggttcc

SEQ ID NO: 41 - DNA, 180 bp, Artificial Sequence
  Synthetic Construct
atgaaaataacatctaccattattcaaacaccttttccatttgagaataataattctcatgctggcatagtaacggagcccattctcggtaagttaataggtcaggggtcgacagcagaaatctttgaagatgtgaatgattcatctgctggtggaggtggctccggcggcggaggttcc

SEQ ID NO: 42 - DNA, 180 bp, Artificial Sequence
  Synthetic Construct
atgaaaccgatcaacaatcattctttttttcgttccctttgtggcttatcatgtatatctcgtttatcggtagaagaacagtgtaccagagattaccaccgcatctgggatgactgggctagggaaggaacaacaacagaaaatcgcatcggtggaggtggctccggcggcggaggttcc

SEQ ID NO: 43 - DNA, 180 bp, Artificial Sequence
  Synthetic Construct
atgttctctgtaaataatacacactcatcagtttcttgctccccctctattaactcaaactcaaccagtaatgaacattatctgagaatcctgactgaatgggaaaagaactcttctcccggggaagagcgaggcattgcttttaacagaggtggaggtggctccggcggcggaggttcc

SEQ ID NO: 44 - DNA, 162 bp, Artificial Sequence
  Synthetic Construct
atgcatatccaatcgcttcaacagtccccgtcttttgctgttgaattgcaccaggcagccagtgggcgtttaggccagattgaagcacggcaagtggccaccccgagcgaagcgcagcagctggcgcagcgccaggatgcgccaaaaggtgagggcctgctg

SEQ ID NO: 45 - DNA, 150 bp, Artificial Sequence
  Synthetic Construct
atgaaaatctcaagctttatttcgacttcactgccactgccgacgtctgtgtccggttcgagcagcgttggcgaaatgagtgggcgctccgtctctcagcaaaccagtgatcagtatgccaacaatttagcgggccgtaccgagagcccg

SEQ ID NO: 46 - protein, 88 aa, Escherichia coli
MALLDFFLSRKKNTANIAKERLQIIVAERRRSDAEPHYLPQLRKDILEVICKYVQIDPEMVTVQLEQKDGDISILELNVTLPEAEELK

SEQ ID NO: 47 - protein, 231 aa, Escherichia coli
MSNTPIELKGSSFTLSVVHLHEAEPKVIHQALEDKIAQAPAFLKHAPVVLNVSALEDPVNWSAMHKAVSATGLRVIGVSGCKDAQLKAEIEKMGLPILTEGKEKAPRPAPTPQAPAQNTTPVTKTRLIDTPVRSGQRIYAPQCDLIVTSHVSAGAELIADGNIHVYGMMRGRALAGASGDRETQIFCTNLMAELVSIAGEYWLSDQIPAEFYGKAARLQLVENALTVQPLN

SEQ ID NO: 48 - protein, 270 aa, Escherichia coli
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 49 - protein, 164 aa, Bacillus subtilis
MPLTPNDIHNKTFTKSFRGYDEDEVNEFLAQVRKDYEIVLRKKTELEAKVNELDERIGHFANIEETLNKSILVAQEAAEDVKRNSQKEAKLIVREAEKNADRIINESLSKSRKIAMEIEELKKQSKVFRTRFQMLIEAQLDLLKNDDWDHLLEYEVDAVFEEKE

SEQ ID NO: 50 - DNA, 9299 bp, Artificial Sequence; identical to SEQ ID NO: 11
  Synthetic Construct
  misc_feature (7845)..(7845): n is a, g, c, or t

SEQ ID NO: 51 - DNA, 3578 bp, Artificial Sequence; identical to SEQ ID NO: 14
  Synthetic Construct

SEQ ID NO: 52 - DNA, 3589 bp, Artificial Sequence
  Synthetic Construct
gatgataaggaacagagacgggaccaaaacgaaaaaaggcccccctttcgggaggcctcttttctggaatttggtaccgaggcattcaattagcgatggcgacttgctcatcaccggaacggaccttacttggcgtagtgccaaagcgacgtcgataactttgagtaaagtaagattggctggaaaatcccgcttccattgccacgtcgacaatgctcattttgctcgtcagcaaaagcttgtgtgcgtgcaataagcgacgctcactgatccaagctcttggagagatgccatagtgctcattaaaaagctctttaaaggttgtgagactggcaccaaattcttgagcataatccgtcagcttccactctttcaaatagtgctgctccataaagcggcgtaagcggtctgaggttcggttagtttgctgcgatagcaaggcatagaggacttcgcccccttgactgtgcagcaatagaatcagcatttcttcgtacttcaaacgcatgatggcgtctgggtacgactgctcctccagcgttttcatgccggaaagcacttgctcaatgagaatgttcaaaccaaaagaaaacaaacccgagttgagtttgtccacttttggcaacgtcatgagaagatcgctgtgtttttgcagtaattggcttactagctcattgtcgaattcaatgatagatgagtgataattcccaccctgtggtgagtactctaacaagtagtcaccttgagagagaaataccccactcccggcaacgacatctatggtgcaatcgccagtttgaactcgtaattgaccattgtgaacgacaacaatatgagattggtctgagtgaaatacttcgtcatgtttttcgtagtgagaaaaactgcgaatttttcgcaagctagagccgaccgtttctgtgtttagttggcctgacacatccattttctacccttccttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagcagtgctcttgtgggtccgattcgcca

SEQ ID NO: 53 - DNA, 4816 bp, Artificial Sequence
  Synthetic Construct
gatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattaattcgcgttggccgattcattaatgctggtaatctatgtatcctggcaactccgagcccttccaccgagacgaaataataaaaaagccggattaataatctggctttttatattctctgattttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagcgctcaacgggtgtgcttcccgttctgatgagtccgtgaggacgaaagcgcctctacaaataattttgtttaaggaagggtagaaaatggatgtgtcaggccaactaaacacagaaacggtcggctctagcttgcgaaaaattcgcagtttttctcactacgaaaaacatgacgaagtatttcactcagaccaatctcatattgttgtcgttcacaatggtcaattacgagttcaaactggcgattgcaccatagatgtcgttgccgggagtggggtatttctctctcaaggtgactacttgttagagtactcaccacagggtgggaattatcactcatctatcattgaattcgacaatgagctagtaagccaattactgcaaaaacacagcgatcttctcatgacgttgccaaaagtggacaaactcaactcgggtttgttttcttttggtttgaacattctcattgagcaagtgctttccggcatgaaaacgctggaggagcagtcgtacccagacgccatcatgcgtttgaagtacgaagaaatgctgattctattgctgcacagtcaagggggcgaagtcctctatgccttgctatcgcagcaaactaaccgaacctcagaccgcttacgccgctttatggagcagcactatttgaaagagtggaagctgacggattatgctcaagaatttggtgccagtctcacaacctttaaagagctttttaatgagcactatggcatctctccaagagcttggatcagtgagcgtcgcttattgcacgcacacaagcttttgctgacgagcaaaatgagcattgtcgacgtggcaatggaagcgggattttccagccaatcttactttactcaaagttatcgacgtcgctttggcactacgccaagtaaggtccgttccggtgatgagcaagtcgccatcgctaattgaatgcctcggtaccaaattccagaaaagaggcctcccgaaaggggggccttttttcgttttggtcccgtctctgttccttatcatctggcgaatcggacccacaagagcactgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgcctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataataacgtaaaaacccgcttcggcgggtttttttatgcaagacgctggctctgacatcgagtagttcagtagcggaagtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcct

SEQ ID NO: 54 - DNA, 8089 bp, Artificial Sequence
  Synthetic Construct
  misc_feature (6635)..(6635): n is a, g, c, or t
gatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattccacatgtggaattccacatgtggaattgtgagcggataacaatttgtggaatccccagcccggaaatcaaaagtaaacgccgtcagcttcatcaagaacttcaagcttctaatcaacgcgaaaacgttaagcgctccaacgtattagtaacgaacccgactcacatcgcggttggtctgtactacaaaaaaggcgaaacaccgttgcctgtcatcactctgatggaaaccgatgcgatggctaaacgcatgattgcaattgcgcgagaagaaggcgtgccagtaatgcaaaaagtgccattagcgcgtgcgctttatgccgacggaaacgttgaccaatacatcccaagcgaattaattgaagccacggcagaagtgctccgttggctggcatcattggaatctgatggtactcaaagatagttttattcaaagggcaaagtacgtcgctttgtccttaataaaactcaggttgatttaacatttctagcacggatacgtctgcagaaagcacgtatgatttacttgtaaacctaaagctcagctccattagccatttgaatgaataatcctattgtgcttgttacgaattcggcggacgagtcatgaaaacagagtgactcaggccgttttgatgctgttatttttcatcattcggcgcgattaatatgctaaagatggtcggatttttttcgttttaagacgtttgaaaacaacaaagctcagcaatgtgctgagctttgtgttcgattgatttggcgatgatgccctactctggtgacgtcattttccgctcactgagttgtttcaccatcgcttgctttaacgcctgaatctgtccatcgatactggcgtcaatgatgccgacttcggtttccagtatgcagccaccgtttttgaggcgcgcatcggcaaccacatcgacataacccacttcaggaaaatcactcaaaacgccagcaactttctctctcacgtcaaccacttgctcaggatgaacgtgtaagatcacttgcttttgattactcaccaattgcagcggggagagctcaggttacccgcatgcaagatctatctagaagggccccactagtgacgcgtactcgagggtcgacggtatcgataagcttgatatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaargaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagwttgctcagctctcccgtggaggtaataattgacgatatgatcatttattctgcctcccagagcctgataaaaacggttagcgcttcgttaatacagatgtaggtgttccacagggtagccagcagcatcctgcgatgcagatcatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctatnggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagag